(19) United States
(12) Patent Application Publication
(10) Pub. No.: US 2025/0170223 A1
(43) Pub. Date: May 29, 2025

(54) TREATMENT OF ENPP1 DEFICIENCY AND ABCC6 DEFICIENCY

(71) Applicant: Inozyme Pharma, Inc., Boston, MA (US)

(72) Inventors: Pedro Huertas, Boston, MA (US); Deborah Wenkert, Boston, MA (US)

(21) Appl. No.: 18/904,807

(22) Filed: Oct. 2, 2024

Related U.S. Application Data
(63) Continuation of application No. PCT/US23/65340, filed on Apr. 4, 2023.
(60) Provisional application No. 63/327,215, filed on Apr. 4, 2022.

(51) Int. Cl. A61K 38/46; A61P 43/00

(52) U.S. Cl. CPC A61K 38/46; A61P 43/00; C12Y 306/01009

(57) ABSTRACT

The present disclosure provides, among other things, specific doses of an ENPP1 agent for in vivo treatment of an ENPP1 deficiency, such as for treatment of Generalized Arterial Calcification of Infancy (GACI), Autosomal Recessive Hypophosphatemic Rickets 2 (ARHR2), and other diseases resulting from pathological calcification, ENPP1 deficiency, ABCC6 deficiency such as diseases or disorders involving ectopic calcification of soft tissue in a subject.

### CROSS REFERENCE TO RELATED APPLICATIONS

This application is a continuation application of International Patent Application No. PCT/US2023/065340, entitled “TREATMENT OF ENPP1 DEFICIENCY AND ABCC6 DEFICIENCY”, filed on Apr. 4, 2023, which claims priority to U.S. Provisional Patent Application No. 63/327,215 entitled “TREATMENT OF ENPP1 DEFICIENCY AND ABCC6 DEFICIENCY” filed on Apr. 4, 2022, each of which are hereby incorporated by reference in their entirety.

### FIELD

The field of the invention relates to treatment of ENPP1 deficiency and ABCC6 deficiency by enzyme replacement.

### SEQUENCE LISTING

This application contains a Sequence Listing which has been submitted electronically as a WIPO Standard ST.26 XML file via Patent Center, created on Oct. 2, 2024, is entitled “4427-12401.xml” and is 124 KB in size. The sequence listing is incorporated herein by reference in its entirety.

### BACKGROUND

Calcification in biological systems is a complex process by which calcium salts are maintained at higher concentrations in noncirculating matrices than in regional circulating humoral or other mobile fluids. The principal result of normal calcification is the concentration of calcium and associated inorganic salts in crystalline patterns of similar arrangement and chemical composition in specialized intercellular matrices, all of which might vary among the different species. The net effect of pathological calcification is the concentration of calcium and associated inorganic salts with a greater-than-normal range in chemical composition or diversity of pattern, not only in these specialized matrices but also in other intercellular, extracellular, and cellular materials leading to several disease states.

Some common examples of disease states of pathological calcification include but are not limited to kidney and bladder stones, dental pulp stones, gall stones, salivary gland stones, chronic calculous prostatitis, testicular microliths, calcification in hemodialysis patients, atherosclerosis, malacoplakia, scleroderma (systemic sclerosis), calcinosis cutis, calcific aortic stenosis, calcific tendonitis, synovitis and arthritis, diffuse interstitial skeletal hyperostosis, juvenile dermatomyositis, Generalized Arterial Calcification of Infancy (GACI), Ossification of the Posterior Longitudinal Ligament (OPLL), hypophosphatemic rickets, osteoarthritis, calcification of atherosclerotic plaques, Chronic Kidney Disease (CKD), End Stage Renal Disease (ESRD), Pseudoxanthoma elasticum (PXE), ankylosing spondylitis, hardening of the arteries, calciphylaxis, and systemic lupus erythematosus.

ENPP1 Deficiency is a rare, genetic disorder caused by inactivating mutations in the ENPP1 gene that encodes the ENPP1 enzyme. ENPP1 is an integral transmembrane protein whose extracellular domains carry pyrophosphatase and phosphodiesterase activities. ENPP1 converts extracellular ATP to inorganic pyrophosphate (PPi) and AMP.

ENPP1 Deficiency causes hypopyrophosphatemia and hypoadenosinemia which, in turn, leads to ectopic (especially arterial) calcification (described in literature as Generalized Arterial Calcification of Infancy [GACI]), skeletal dysfunction secondary to rickets and osteomalacia (described in literature as Autosomal Recessive Hypophosphatemic Rickets 2 [ARHR2]) and occlusive neo-intimal proliferation. Beyond symptomatic and palliative interventions, no targeted therapy exists for this disease. Thus, ENPP1 Deficiency has a high unmet medical need. Infants with ENPP1 Deficiency have high mortality in the first 0 to 6 months of life and children and adults with ENPP1 Deficiency experience ongoing risk for organ calcification and dysfunction, debilitating rickets that progresses to osteomalacia in adulthood with severe bone and joint pain, fatigue, muscle weakness, and repeated bone fractures, all symptoms that lead to poor quality of life and function. Hypopyrophosphatemia causes a reactive increase in fibroblast growth factor 23 (FGF23) leading to hypophosphaturia (the ENPP1 Deficiency “biochemical axis”), an essential feature in the pathophysiology of ENPP1 Deficiency.

ENPP1 Deficiency is characterized biochemically by low plasma PPi levels and clinically characterized by vascular calcification in infants (GACI Type phenotype) and rickets (ARHR2 phenotype) post-infancy and intimal proliferation. GACI (generalized arterial calcification of infants) is a severe disease occurring in infants and involving extensive arterial calcification (Albright, et al., 2015, Nature Comm. 10006).

ABCC6 deficiency is a rare, inherited, genetic inborn error of metabolism caused by mutations in the ABCC6 gene. ABCC6 deficiency is inherited as a recessive trait in which the genetic mutations result in decreased or absent activity of the ABCC6 protein (also known as MRP6 (multi-drug resistance protein 6), ATP-binding cassette sub-family C member 6 (ABCC6) and multi-specific organic anion transporter E (MOAT-E)). The deficiency leads to low plasma levels of PPi and is associated with pathological mineralization in blood vessels and soft tissues throughout the body, resulting in significant morbidity, including blindness, life-threatening cardiovascular complications, and skin calcification throughout the body, resulting in significant morbidity, including blindness, life-threatening cardiovascular complications, and skin calcification. The pathological mineralization associated with ABCC6 deficiency is the result of ectopic calcification in elastic fibers, a component of connective tissue, which provides strength and flexibility to structures throughout the body. Ectopic calcification can affect function in elastic fibers in the eyes, blood vessels, and skin, and less frequently in other areas such as the digestive tract.

Mutations in ABCC6 cause pseudoxanthoma elasticum (PXE).[9] The most common mutations, R1141X and 23-29del, account for about 25% of identified mutations. Le Saux 0, et al. (2001). “A spectrum of ABCC6 mutations is responsible for pseudoxanthoma elasticum”. Am. J. Hum. Genet. 69 (4): 749-64. Pfendner E G, et al. (2007). “Mutation Detection in the ABCC6 Gene and Genotype-Phenotype Analysis in a Large International Case Series Affected by Pseudoxanthoma Elasticum”. Journal of Medical Genetics. 44 (10): 621-8.

Premature atherosclerosis is also associated with mutations in the ABCC6 gene, even in those without PXE. Trip M D, et al. (2002). “Frequent mutation in the ABCC6 gene (R1141X) is associated with a strong increase in the prevalence of coronary artery disease”. Circulation. 106 (7): 773-5.

Deficiency of ABCC6 in mouse models of ischemia leads to larger infarcts, which can be rescued by ABCC6 overexpression. Mungrue I N, et al. (2011). “ABCC6 deficiency causes increased infarct size and apoptosis in a mouse cardiac ischemia-reperfusion model”. Arterioscler Thromb Vase Biol. 31 (12): 2806-12.

Some infants with ABCC6 deficiency are diagnosed with a vascular calcification condition resembling the acute infantile form of ENPP1 deficiency. In older patients, ABCC6 deficiency presents as pseudoxanthoma elasticum, or PXE, a rare disorder in which individuals develop calcification of soft connective tissues, including in the eyes, cardiovascular system, and skin. Individuals with PXE often have abnormalities in the eyes, such as a change in the pigmented cells of the retina or angioid streaks that occur when tiny cracks form in Bruch's membrane, the elastic membrane beneath the retina. Choroidal neovascularization—subsequent bleeding and scarring of the retina—may also occur, which, together with the damage to Bruch's membrane, can cause vision loss. A recent report stated that 37 percent of PXE patients over the age of 50 experienced visual impairment and 15 percent were legally blind. (Risseeuw S, Ossewaarde-van Norel J, Klayer C C W, Colijn J M, Imhof S M, van Leeuwen R. Visual acuity in pseudoxanthoma elasticum. Retina. 2019; 39(8):1580-1587). Pathological mineralization of the vessels that carry blood from the heart to the rest of the body may cause other signs and symptoms of PXE. Ectopic calcification narrows blood vessels, particularly in the lower extremities, and leads to claudication, a condition characterized by cramping and pain during exercise due to decreased blood flow to the arms and legs. Individuals with PXE may also have yellowish bumps called papules on the neck, underarms, and other areas of the skin surrounding the joints. These papules are painful, can impair joint movement, and indicate a general, systemic, pathological process of soft tissue calcification.

PPi is modulator of bone mineralization and a potent inhibitor of calcium hydroxyapatite crystal deposition and is essential for prevention of harmful soft tissue calcification.

PPi functions as a potent inhibitor of ectopic tissue mineralization by binding to nascent hydroxyapatite (HA) crystals, thereby preventing the future growth of these crystals. ENPP1 generates PPi via hydrolysis of nucleotide triphosphates (NTPs), Progressive Ankylosis Protein (ANK) transports intracellular PPi into the extracellular space, and Tissue Non-specific Alkaline Phosphatase (TNAP) removes PPi via direct hydrolysis of PPi into Pi.

Ectopic tissue mineralization is associated with numerous human diseases, including chronic joint disease and acutely fatal neonatal syndromes. To prevent unwanted tissue calcification, factors that promote and inhibit tissue mineralization must be kept in tight balance. The balance of extracellular inorganic pyrophosphate (PPi) and phosphate (Pi) is an important regulator of ectopic tissue mineralization. The activity of the three extracellular enzymes—TNAP, ANK, and ENPP1—tightly control the concentration of Pi and PPi in mammals at μ mM and 2-3 μM respectively. PPi is a regulator of biomineralization, inhibiting the formation of basic calcium phosphate from amorphous calcium phosphate.

ENPP1 polypeptides have been shown to be effective in treating certain diseases of ectopic tissue calcification. ENPP1-Fc has been shown to reduce generalized arterial calcifications in a mouse model for GACI (generalized arterial calcification of infants), which is a severe disease occurring in infants and involving extensive arterial calcification (Albright, et al., 2015, Nature Comm. 10006). Fusion proteins of ENPP1 also have been described to treat diseases of severe tissue calcification (see, e.g., PCT Application Publication Nos. WO 2014/126965 and WO 2016/187408), and a fusion protein of ENPP1 comprising a negatively charged bone-targeting domain has been described to treat GACI (PCT Application Publication Nos. WO 2011/113027 and WO 2012/125182).

Currently no clinically approved treatments exist for these ultra-rare genetic, chronic, progressive, and life-threatening diseases in which patients experience devastating effects on multiple systems of the body, leading to life-threatening or debilitating complications.

### SUMMARY

The disclosure relates to administration of an ENPP1 agent to a subject at a dose of about 0.2 mg per kilogram of the subject, about 0.4 mg per kilogram of the subject, about 0.6 mg per kilogram of the subject, about 1.2 mg per kilogram of the subject or about 1.8 mg per kilogram of the subject.

In an aspect, the disclosure relates to administering to a subject having an ENPP1 deficiency or to a subject having an ABCC6 deficiency, an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject in order to restore a physiological level of ENPP1 protein and/or activity in the plasma or tissues of the subject. A physiological level of ENPP1 activity in the plasma or tissues, as used herein, is an amount or concentration of ENPP1 activity sufficient to achieve and maintain a physiological level of PPi in human serum.

In an aspect, the disclosure relates to administering to a subject having an ENPP1 deficiency or to a subject having an ABCC6 deficiency, an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject in order to restore a physiological level of Pi and/or PPi in the plasma of the subject. The physiological level of Pi and PPi in human serum (and in mammals generally) is 1-3 mM and 2-3 μM respectively.

In an aspect, the disclosure relates to a method for preventing progression of or reducing vascular calcification in a subject with ENPP1 Deficiency or in a subject having an ABCC6 deficiency, the method comprising: to thereby prevent the progression of or reduce vascular calcification in the subject, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby prevent the progression of or reduce vascular calcification in the subject.

In an aspect, the disclosure relates to a method for preventing the progression of or reducing pathological calcification in a subject with ENPP1 Deficiency or in a subject having an ABCC6 deficiency, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby prevent the progression of or reduce pathological calcification in the subject.

In an aspect, the disclosure relates to a method for preventing the progression of or reducing tissue calcification in a subject with ENPP1 Deficiency or a subject having an ABCC6 deficiency, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby prevent the progression of or reduce tissue calcification in the subject.

In an aspect, the disclosure relates to a method for preventing the progression of or reducing pathological ossification in a subject with ENPP1 Deficiency or in a subject having an ABCC6 deficiency, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby prevent the progression of or reduce tissue calcification in the subject.

In an aspect, the disclosure relates to a method for increasing circulating pyrophosphate (PPi) in a subject with ENPP1 Deficiency or in a subject having an ABCC6 deficiency, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby increase circulating PPi in the subject.

In an aspect, the disclosure relates to a method for increasing pyrophosphatase activity in a subject with ENPP1 Deficiency or in a subject having an ABCC6 deficiency, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby increase circulating PPi in the subject.

In an aspect, the disclosure relates to a method for ameliorating one or more symptoms of ENPP1 Deficiency in a subject or one or more symptoms of ABCC6 deficiency in a subject, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby ameliorate one or more symptoms of ENPP1 Deficiency or one or more symptoms of ABCC6 Deficiency in the subject.

In an aspect, the disclosure relates to a method for treating a subject with ENPP1 Deficiency or a subject having an ABCC6 deficiency, the method comprising: administering to the subject an ENPP1 agent at a dose of about 0.2 mg per kilogram of the subject, about 0.4 mg per kilogram of the subject, about 0.6 mg per kilogram of the subject, about 1.2 mg per kilogram of the subject, or about 1.8 mg per kilogram of the subject, to thereby treat the subject.

In an aspect, the disclosure relates to methods in which the administered dose is about 0.2 mg per kilogram of the subject.

In some embodiments, the disclosure relates to methods in which the administered dose is 0.2 (±1−10%) mg per kilogram of the subject. For example, the dose can be 0.22 mg/kg (0.2+10% mg/kg), or 0.18 mg/kg (0.2−10% mg/kg), or 0.21 mg/kg (0.2+5% mg/kg), or 0.19 mg/kg (0.2−5% mg/kg) or 0.202 mg/kg (0.2+1% mg/kg) or 0.198 mg/kg (0.2−1% mg/kg) of the subject.

In an aspect, the disclosure relates to methods in which the administered dose is about 0.4 mg per kilogram of the subject.

In some embodiments, the disclosure relates to methods in which the administered dose is 0.4 (±1−10%) mg per kilogram of the subject. For example, the dose can be 0.44 mg/kg (0.4+10% mg/kg), or 0.36 mg/kg (0.4−10% mg/kg), or 0.42 mg/kg (0.4+5% mg/kg), or 0.38 mg/kg (0.4−5% mg/kg) or 0.404 mg/kg (0.4+1% mg/kg) or 0.396 mg/kg (0.4−1% mg/kg) of the subject.

In an aspect, the disclosure relates to methods in which the administered is about 0.6 mg per kilogram of the subject.

In some embodiments, the disclosure relates to methods in which the administered dose is 0.6 (±1−5%) mg per kilogram of the subject. For example, the dose can be 0.63 mg/kg (0.6+5% mg/kg), or 0.57 mg/kg (0.6−5% mg/kg), or 0.624 mg/kg (0.6+4% mg/kg), or 0.576 mg/kg (0.6−4% mg/kg) or 0.618 mg/kg (0.6+3% mg/kg) or 0.582 mg/kg (0.6−3% mg/kg) or 0.612 mg/kg (0.6+2% mg/kg) or 0.588 mg/kg (0.6−2% mg/kg) or 0.606 mg/kg (0.6+1% mg/kg) or 0.594 mg/kg (0.6−1% mg/kg) of the subject.

In an aspect, the disclosure relates to methods in which the administered is about 1.2 mg per kilogram of the subject.

In some embodiments, the disclosure relates to methods in which the administered dose is 1.2 (1−5%) mg per kilogram of the subject. For example, the dose can be 1.26 mg/kg (1.2+5% mg/kg), or 1.14 mg/kg (1.2−5% mg/kg), or 1.248 mg/kg (1.2+4% mg/kg), or 1.152 mg/kg (1.2−4% mg/kg) or 1.236 mg/kg (1.2+3% mg/kg) or 1.164 mg/kg (1.2−3% mg/kg) or 1.176 mg/kg (1.2+2% mg/kg) or 1.224 mg/kg (1.2−2% mg/kg) or 1.212 mg/kg (1.2+1% mg/kg) or 1.188 mg/kg (1.2−1% mg/kg) of the subject.

In an aspect, the disclosure relates to methods in which the administered is about 1.8 mg per kilogram of the subject.

In some embodiments, the disclosure relates to methods in which the administered dose is 1.8 (±1−5%) mg per kilogram of the subject. For example, the dose can be 1.89 mg/kg (1.8+5% mg/kg), or 1.71 mg/kg (1.8−5% mg/kg), or 1.872 mg/kg (1.8+4% mg/kg), or 1.728 mg/kg (1.8−4% mg/kg) or 1.854 mg/kg (1.8+3% mg/kg) or 1.746 mg/kg (1.8−3% mg/kg) or 1.836 mg/kg (1.8+2% mg/kg) or 1.764 mg/kg (1.8−2% mg/kg) or 1.818 mg/kg (1.8+1% mg/kg) or 1.782 mg/kg (1.8−1% mg/kg) of the subject.

In yet another aspect, the disclosure relates to a method for treating a subject with ENPP1 Deficiency or a subject with ABCC6 deficiency, the method comprising administering to the subject an ENPP1 agent in an amount effective to treat, or otherwise ameliorate one or more symptoms associated with, the subject's ENPP1 deficiency or the subject's ABCC6 deficiency.

In some embodiments of any of the methods described herein, the subject can be, e.g., one who has discontinued (e.g., at least 14 days prior to treatment with the ENPP1 agent) treatment with (or is otherwise not receiving at the time of treatment with the ENPP1 agent) one or more (or all) of the following: a bisphosphonate, an anti-FGF23 antibody or FGF23 antagonist, a calcimimetic, an antacid, a corticosteroid, or a PTH suppressor.

In some embodiments of any of the methods described herein, the subject is one who has been pre-treated with one or more statins and/or one or more proprotein convertase subtilisin/kexin type 9 (PCSK9) inhibitors. Preferably, such pre-treatment include consistent dosage and frequency of the one or more statins and/or one or more PCSK9 inhibitors for 3 or more years prior to treatment according to methods described herein.

In some embodiments of any of the methods described herein, the subject has not been diagnosed with a malignancy within 5 years prior to treatment according to methods described herein, with the exception of non-melanoma skin cancers and/or cervical carcinoma in situ.

In an aspect, the disclosure relates to methods in which the ENPP1 agent is administered at least one time per week or once weekly.

In an aspect, the disclosure relates to methods in which the ENPP1 agent is administered at least two times per week.

In an aspect, the disclosure relates to methods in which the ENPP1 agent is administered once every two weeks.

In an aspect, the disclosure relates to methods in which the ENPP1 agent is administered once every three weeks.

In an aspect, the disclosure relates to methods in which the ENPP1 agent is administered once every month.

In an aspect, the disclosure relates to methods in which the ENPP1 agent is administered to the subject at least two times per week following an initial dose.

In an aspect, the disclosure relates to methods in which the ENPP1 agent is administered subcutaneously.

In an aspect, the disclosure relates to methods in which the ENPP1 agent is self-administered.

In an aspect, the disclosure relates to methods in which the ENPP1 agent is administered under a dosing regimen comprising: (a) an initial dose of about 0.2 mg per kilogram of the subject, about 0.6 mg per kilogram of the subject, or about 1.8 mg per kilogram of the subject and (b) about seven days after the initial dose, twice weekly administration of maintenance doses of the ENPP1 agent of about 0.2 mg per kilogram of the subject, about 0.6 mg per kilogram of the subject, or about 1.8 mg per kilogram of the subject.

In an aspect, the disclosure relates to methods in which the initial dose of the ENPP1 agent and the maintenance dose of the ENPP1 agent are the same amount.

In an aspect, the disclosure relates to methods in which the administered ENPP1 agent comprises the catalytic domain of ENPP1.

In an aspect, the disclosure relates to methods in which the administered ENPP1 agent comprises the nuclease domain of ENPP1.

In an aspect, the disclosure relates to methods in which the administered ENPP1 agent comprises the extracellular domain of ENPP1.

In an aspect, the disclosure relates to methods in which the administered ENPP1 agent comprises the catalytic and nuclease domains of ENPP1.

In an aspect, the disclosure relates to methods in which the administered ENPP1 agent comprises a heterologous moiety; the heterologous moiety may be a polypeptide.

In an aspect, the disclosure relates to methods in which the heterologous moiety increases the circulating half-life of the ENPP1 agent relative to the circulating half-life of the ENPP1 agent lacking the heterologous moiety.

In an aspect, the disclosure relates to methods in which the heterologous moiety comprises the Fc region of an immunoglobulin molecule; the immunoglobulin molecules may be a human immunoglobulin molecule; the immunoglobulin molecule may be an IgG1.

In an aspect, the disclosure relates to methods in which the heterologous moiety comprises albumin; the heterologous moiety comprises serum albumin; the heterologous moiety comprises human serum albumin.

In an aspect, the disclosed ENPP1 agent and methods of its use relates to an ENPP1 agent comprising amino acid residues 99 (PSCAKE) to 925(QED) of SEQ ID NO:1.

In an aspect, the disclosed ENPP1 agent and methods of its use relates to an ENPP1 agent comprising amino acid residues 1 (FTAGLKPSCAKE) to 833 (QED) of SEQ ID NO:3.

In an aspect, the disclosed ENPP1 agent and methods of its use relates to an ENPP1 agent comprising the amino acid sequence depicted in SEQ ID NO:2.

In an aspect, the disclosed ENPP1 agent and methods of its use relates to an ENPP1 agent comprising the amino acid sequence depicted in SEQ ID NO:3.

In an aspect, the disclosed ENPP1 agent and methods of its use relates to an ENPP1 agent comprising the amino acid sequence depicted in SEQ ID NO:4.

In an aspect, the disclosed ENPP1 agent and methods of its use relates to an ENPP1 agent comprising the amino acid sequence depicted in SEQ ID NO:5.

In an aspect, the disclosure relates to methods in which the subject has or is suspected of having generalized arterial calcification of infancy (GACI).

In an aspect, the disclosure relates to methods in which the subject has or is suspected of having autosomal recessive hypophosphatemic rickets type 2 (ARHR2).

In an aspect, the disclosure relates to methods in which the subject has or is suspected of having Pseudoxanthoma elasticum (PXE).

In an aspect, the disclosure relates to methods in which the subject having ABCC6 deficiency and exhibits symptoms similar to a subject having autosomal recessive hypophosphatemic rickets type 2 (ARHR2) or generalized arterial calcification of infancy (GACI).

In an aspect, the disclosure relates to administering to a subject having pathological calcification an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject in order to restore a physiological level of Pi and/or PPi in the plasma of the subject. The physiological level of Pi and PPi in human serum (and in mammals generally) is 1-3 mM and 2-3 μM respectively.

In an aspect, the disclosure relates to a method for preventing progression of or reducing vascular calcification in a subject having pathological calcification, the method comprising: to thereby prevent the progression of or reduce vascular calcification in the subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce vascular calcification in the subject.

In an aspect, the disclosure relates to a method for preventing the progression of or reducing pathological calcification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce pathological calcification in the subject.

In an aspect, the disclosure relates to a method for preventing the progression of or reducing tissue calcification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject

In an aspect, the disclosure relates to a method for preventing the progression of or reducing pathological ossification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject.

In an aspect, the disclosure relates to a method for increasing circulating pyrophosphate (PPi) in a subject with pathological calcification, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase circulating PPi in the subject.

In an aspect, the disclosure relates to a method for increasing pyrophosphatase activity in a subject with pathological calcification, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase circulating PPi in the subject.

In an aspect, the disclosure relates to a method for ameliorating one or more symptoms of pathological calcification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby ameliorate one or more symptoms of pathological calcification in the subject.

In an aspect, the disclosure relates to a method for treating a subject with pathological calcification, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat the subject.

In an aspect, the disclosure relates to methods in which the administered dose is about 0.2 mg per kilogram of the subject.

In some embodiments, the disclosure relates to methods in which the administered dose is 0.2 (±1−10%) mg per kilogram of the subject. For example, the dose can be 0.22 mg/kg (0.2+10% mg/kg), or 0.18 mg/kg (0.2−10% mg/kg), or 0.21 mg/kg (0.2+5% mg/kg), or 0.19 mg/kg (0.2−5% mg/kg) or 0.202 mg/kg (0.2+1% mg/kg) or 0.198 mg/kg (0.2−1% mg/kg) of the subject.

In an aspect, the disclosure relates to methods in which the administered is about 0.6 mg per kilogram of the subject.

In some embodiments, the disclosure relates to methods in which the administered dose is 0.6 (±1−5%) mg per kilogram of the subject. For example, the dose can be 0.63 mg/kg (0.6+5% mg/kg), or 0.57 mg/kg (0.6−5% mg/kg), or 0.624 mg/kg (0.6+4% mg/kg), or 0.576 mg/kg (0.6−4% mg/kg) or 0.618 mg/kg (0.6+3% mg/kg) or 0.582 mg/kg (0.6−3% mg/kg) or 0.612 mg/kg (0.6+2% mg/kg) or 0.588 mg/kg (0.6−2% mg/kg) or 0.606 mg/kg (0.6+1% mg/kg) or 0.594 mg/kg (0.6−1% mg/kg) of the subject.

In an aspect, the disclosure relates to methods in which the administered is about 1.8 mg per kilogram of the subject.

In some embodiments, the disclosure relates to methods in which the administered dose is 1.8 (±1−5%) mg per kilogram of the subject. For example, the dose can be 1.89 mg/kg (1.8+5% mg/kg), or 1.71 mg/kg (1.8−5% mg/kg), or 1.872 mg/kg (1.8+4% mg/kg), or 1.728 mg/kg (1.8−4% mg/kg) or 1.854 mg/kg (1.8+3% mg/kg) or 1.746 mg/kg (1.8−3% mg/kg) or 1.836 mg/kg (1.8+2% mg/kg) or 1.764 mg/kg (1.8−2% mg/kg) or 1.818 mg/kg (1.8+1% mg/kg) or 1.782 mg/kg (1.8−1% mg/kg) of the subject.

In yet another aspect, the disclosure relates to a method for treating a subject with pathological calcification, the method comprising administering to the subject an ENPP1 agent in an amount effective to treat, or otherwise ameliorate one or more symptoms associated with, the subject's pathological calcification.

In some embodiments of any of the methods described herein, the subject can be, e.g., one who has discontinued (e.g., at least 14 days prior to treatment with the ENPP1 agent) treatment with (or is otherwise not receiving at the time of treatment with the ENPP1 agent) one or more (or all) of the following: a bisphosphonate, an anti-FGF23 antibody or FGF23 antagonist, a calcimimetic, an antacid, a corticosteroid, or a PTH suppressor.

In an aspect, the disclosure relates to methods in which the ENPP1 agent is administered at least one time per week.

In an aspect, the disclosure relates to methods in which the ENPP1 agent is administered at least two times per week.

In an aspect, the disclosure relates to methods in which the ENPP1 agent is administered to the subject at least two times per week following an initial dose.

In an aspect, the disclosure relates to methods in which the ENPP1 agent is administered subcutaneously.

In an aspect, the disclosure relates to methods in which the ENPP1 agent is self-administered.

In an aspect, the disclosure relates to methods in which the ENPP1 agent is administered under a dosing regimen comprising: (a) an initial dose of about 0.2 mg per kilogram of the subject, about 0.6 mg per kilogram of the subject, or about 1.8 mg per kilogram of the subject and (b) about seven days after the initial dose, twice weekly administration of maintenance doses of the ENPP1 agent of about 0.2 mg per kilogram of the subject, about 0.6 mg per kilogram of the subject, or about 1.8 mg per kilogram of the subject.

In an aspect, the disclosure relates to methods in which the initial dose of the ENPP1 agent and the maintenance dose of the ENPP1 agent are the same amount.

In an aspect, the disclosure relates to methods in which the administered ENPP1 agent comprises the catalytic domain of ENPP1.

In an aspect, the disclosure relates to methods in which the administered ENPP1 agent comprises the nuclease domain of ENPP1.

In an aspect, the disclosure relates to methods in which the administered ENPP1 agent comprises the extracellular domain of ENPP1.

In an aspect, the disclosure relates to methods in which the administered ENPP1 agent comprises the catalytic and nuclease domains of ENPP1.

In an aspect, the disclosure relates to methods in which the administered ENPP1 agent comprises a heterologous moiety; the heterologous moiety may be a polypeptide.

In an aspect, the disclosure relates to methods in which the heterologous moiety increases the circulating half-life of the ENPP1 agent relative to the circulating half-life of the ENPP1 agent lacking the heterologous moiety.

In an aspect, the disclosure relates to methods in which the heterologous moiety comprises the Fc region of an immunoglobulin molecule; the immunoglobulin molecules may be a human immunoglobulin molecule; the immunoglobulin molecule may be an IgG1.

In an aspect, the disclosure relates to methods in which the heterologous moiety comprises albumin; the heterologous moiety comprises serum albumin; the heterologous moiety comprises human serum albumin.

In an aspect, the disclosed ENPP1 agent and methods of its use relates to an ENPP1 agent comprising amino acid residues 99 (PSCAKE) to 925 (QED) of SEQ ID NO:1.

In an aspect, the disclosed ENPP1 agent and methods of its use relates to an ENPP1 agent comprising amino acid residues 1 (FTAGLKPSCAKE) to 833 (QED) of SEQ ID NO:3.

In an aspect, the disclosed ENPP1 agent and methods of its use relates to an ENPP1 agent comprising the amino acid sequence depicted in SEQ ID NO:2.

In an aspect, the disclosed ENPP1 agent and methods of its use relates to an ENPP1 agent comprising the amino acid sequence depicted in SEQ ID NO:3.

In an aspect, the disclosed ENPP1 agent and methods of its use relates to an ENPP1 agent comprising the amino acid sequence depicted in SEQ ID NO:4.

In an aspect, the disclosed ENPP1 agent and methods of its use relates to an ENPP1 agent comprising the amino acid sequence depicted in SEQ ID NO:5.

In an aspect, the disclosure relates to methods in which the subject has or is suspected of having diseases of pathological calcification other than generalized arterial calcification of infancy (GACI).

In an aspect, the disclosure relates to methods in which the subject has or is suspected of having diseases of pathological calcification other than autosomal recessive hypophosphatemic rickets type 2 (ARHR2).

In an aspect, the disclosure relates to methods in which the subject has or is suspected of having diseases of pathological calcification other than Pseudoxanthoma elasticum (PXE).

In an aspect, the disclosure relates to methods in which the subject has or is suspected of having one or more of kidney and bladder stones, dental pulp stones, gall stones, salivary gland stones, chronic calculous prostatitis, testicular microliths, calcification in hemodialysis patients, atherosclerosis, malacoplakia, scleroderma (systemic sclerosis), ARHR2, calcinosis cutis, calcific aortic stenosis, calcific tendonitis, synovitis and arthritis, diffuse interstitial skeletal hyperostosis, juvenile dermatomyositis, Generalized Arterial Calcification of Infancy (GACI), Ossification of the Posterior Longitudinal Ligament (OPLL), hypophosphatemic rickets, osteoarthritis, calcification of atherosclerotic plaques, Chronic Kidney Disease (CKD), End Stage Renal Disease (ESRD), Pseudoxanthoma elasticum (PXE), ankylosing spondylitis, hardening of the arteries, calciphylaxis, and systemic lupus erythematosus.

In some embodiments of any of the methods described herein, the subject is characterized by one or more of the inclusion criteria described herein. For example, a subject described herein can have a plasma PPi concentration of below approximately 1300 nM, e.g., prior to treatment with an ENPP1 agent. In some embodiments, the subject is not characterized by any of the exclusion criteria described herein. For example, the subject can be one who does not have advanced eye disease prior to, or during, treatment with an ENPP1 agent. In some embodiments, the subject does not have advanced eye disease requiring anti-VEGF treatment (for example) prior to, or during, treatment with an ENPP1 agent.

In some embodiments of any of the methods described herein, the subject exhibits no adverse events post administration of said dose. In some embodiments of any of the methods described herein, the subject exhibits low levels of anti-drug antibody (ADA) post administration of said dose. In some embodiments of any of the methods described herein, the subject post administration of said dose exhibits PPi levels that are either equal or greater than the PPi levels of a healthy individual.

### BRIEF DESCRIPTION OF THE DRAWINGS

FIG. 1 presents a schematic overview of ENPP1 Deficiency.

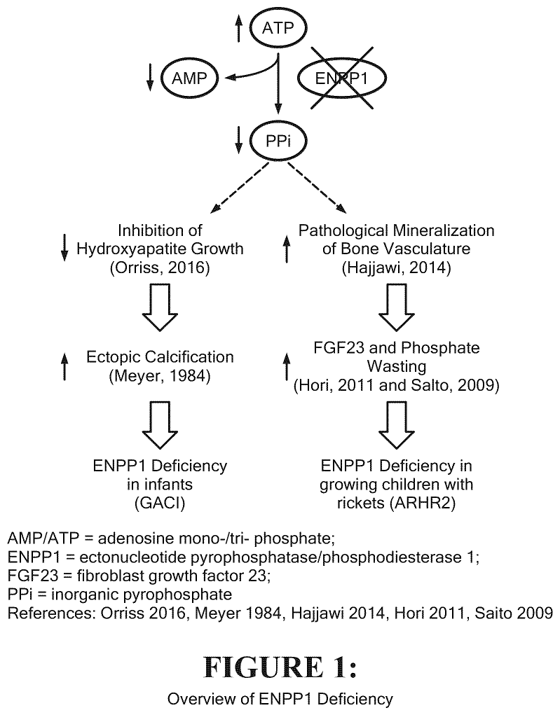

FIG. 2 shows the full, unprocessed amino acid sequence of wild-type ENPP1 precursor protein (SEQ ID NO: 1). The cytosolic and transmembrane regions are underlined. Potential N-glycosylation sites are in bold. PSCAKE (residues 99-104; boxed) is the start of soluble ENPP1 protein portion which includes SMB1 (residues 104-144) and SMB2 (residues 145-189).

FIG. 3 illustrates certain domains of human ENPP1.

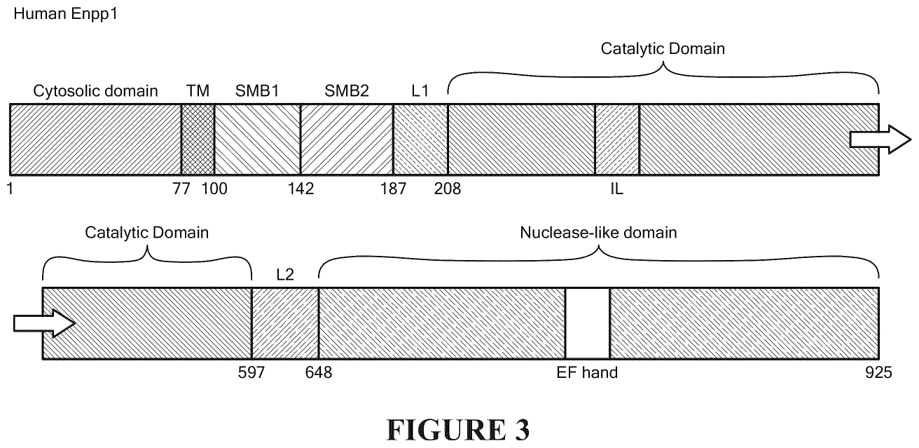

FIG. 4 shows the amino acid sequence of a soluble wild-type ENPP1 polypeptide (SEQ ID NO: 2).

FIG. 5A and FIG. 5B show a multiple sequence alignment of various vertebrate soluble ENPP1 polypeptides and human soluble ENPP1 polypeptide (SEQ ID NOs: 1 and 7-10). The various soluble ENPP1 polypeptides correspond to the following species and represent regions of the specific NCBI accession number: Mouse (NCBI accession NP_001295256.1; SEQ ID NO:7), Cow (NCBI accession NP_001193141; SEQ ID NO: 8), Rabbit (NCBI accession NP_001162404.1; SEQ ID NO:9), Human (NCBI accession NP_006199.2; SEQ ID NO: 1), and Baboon (NCBI accession NP_001076211.2; SEQ ID NO: 10).

FIG. 6 presents a schematic overview of ABCC6 Deficiency.

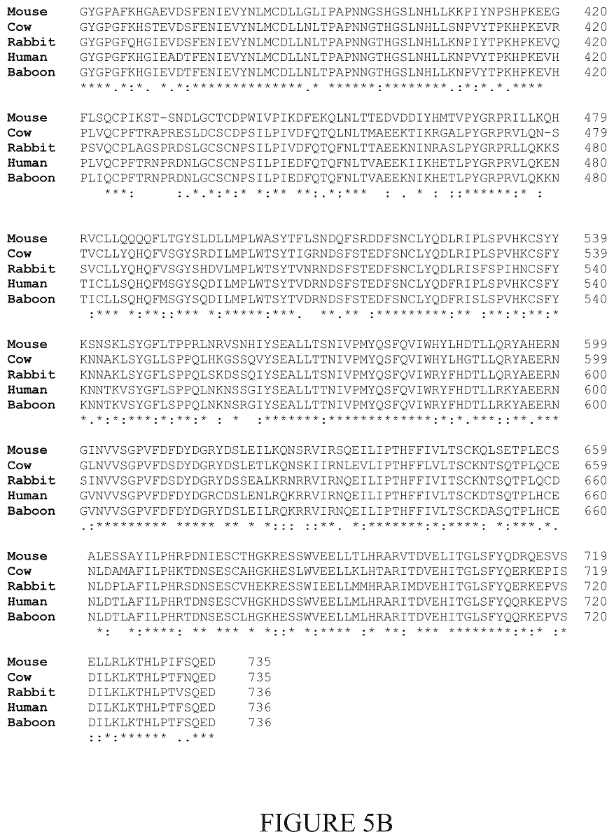

FIG. 7 shows the increase of PPi levels in ENPP1 deficient patients upon administration of ENPP1-Fc at 0.2 mg/kg dose.

FIG. 8 shows the ENPP1 PK data in patients dosed with ENPP1-Fc at 0.2 mg/kg dose for a study period of 32 days.

FIG. 9 shows the ENPP1 activity in patients dosed with ENPP1-Fc at 0.2 mg/kg dose for a study period of 32 days.

FIG. 10 shows the increase of PPi levels in different cohorts of ENPP1 deficient patients upon a single dose administration of ENPP1-Fc at 0.2 mg/kg, 0.6 mg/kg and 1.8 mg/kg for a study period of Days 1-8.

FIG. 11 shows the increase of PPi levels in different cohorts of ENPP1 deficient patients upon twice a week administration of ENPP1-Fc at 0.2 mg/kg, 0.6 mg/kg and 1.8 mg/kg for a study period of Days 11-32.

FIG. 12 shows the increase of PPi levels in different cohorts of ENPP1 deficient patients upon twice a week administration of ENPP1-Fc at 0.2 mg/kg, 0.6 mg/kg and 1.8 mg/kg for an extended study period of days 32-338.

FIG. 13 shows the ENPP1 PK data in different cohorts of patients dosed with ENPP1-Fc at 0.2 mg/kg, 0.6 mg/kg and 1.8 mg/kg dose for a study period of 48 weeks.

FIG. 14 shows the ENPP1 activity in different cohorts of patients dosed with ENPP1-Fc at 0.2 mg/kg, 0.6 mg/kg and 1.8 mg/kg dose for a study period of 48 weeks.

FIG. 15 shows Clinician's global impression change scale in different cohorts of patients dosed with ENPP1-Fc at 0.2 mg/kg, 0.6 mg/kg and 1.8 mg/kg dose for an extended study period.

FIG. 16 shows Patient's global impression change scale in different cohorts of patients dosed with ENPP1-Fc at 0.2 mg/kg, 0.6 mg/kg and 1.8 mg/kg dose for an extended study period.

FIG. 17 shows the safety profile of different cohorts of patients being administered with ENPP1-Fc at 0.2 mg/kg, 0.6 mg/kg and 1.8 mg/kg dose for a study period of 48 weeks.

FIG. 18 shows the immunogenicity profile of different cohorts of patients being administered with ENPP1-Fc at 0.2 mg/kg, 0.6 mg/kg and 1.8 mg/kg dose for a study period of 48 weeks.

### DEFINITIONS

The terms used in this specification generally have their ordinary meanings in the art, within the context of this disclosure and in the specific context where each term is used. Certain terms are discussed below or elsewhere in the specification, to provide additional guidance to the practitioner in describing the compositions and methods of the disclosure and how to make and use them. The scope or meaning of any use of a term will be apparent from the specific context in which the term is used.

An “ENPP1 Deficiency” is characterized by a reduced level of ENPP1 enzymatic activity in serum or plasma of a subject. ENPP1 Deficiency is a rare, genetic disorder caused by inactivating mutations in the ENPP1 gene that encodes the ENPP1 enzyme. ENPP1 is an integral transmembrane protein whose extracellular domains carry pyrophosphatase and phosphodiesterase activities. ENPP1 converts extracellular ATP to inorganic pyrophosphate (PPi) and AMP.

ENPP1 Deficiency causes hypopyrophosphatemia and hypoadenosinemia which, in turn, leads to ectopic (especially arterial) calcification (described in literature as Generalized Arterial Calcification of Infancy [GACI]), skeletal dysfunction secondary to rickets and osteomalacia (described in literature as Autosomal Recessive Hypophosphatemic Rickets 2 [ARHR2]) and occlusive neo-intimal proliferation. Beyond symptomatic and palliative interventions, no targeted therapy exists for this disease. Thus, ENPP1 Deficiency has a high unmet medical need. Infants with ENPP1 Deficiency have high mortality in the first 0 to 6 months of life and children and adults with ENPP1 Deficiency experience ongoing risk for organ calcification and dysfunction, debilitating rickets that progresses to osteomalacia in adulthood with severe bone and joint pain, fatigue, muscle weakness, and repeated bone fractures, all symptoms that lead to poor quality of life and function. Hypopyrophosphatemia causes a reactive increase in fibroblast growth factor 23 (FGF23) leading to hypophosphaturia (the ENPP1 Deficiency “biochemical axis”), an essential feature in the pathophysiology of ENPP1 Deficiency.

ENPP1 Deficiency is characterized biochemically by low plasma PPi levels and clinically characterized by vascular calcification in infants (GACI Type phenotype) and rickets (ARHR2 phenotype) post-infancy and intimal proliferation. GACI (generalized arterial calcification of infants) is a severe disease occurring in infants and involving extensive arterial calcification (Albright, et al., 2015, Nature Comm. 10006).

Reduced ENPP1 enzymatic activity may occur via a reduction in the amount of ENPP1 enzyme present in serum or plasma of a subject relative to the amount of ENPP1 enzyme present in serum or plasma of a normal subject, and/or a reduction in the level of enzymatic activity of ENPP1 detected in serum or plasma of a subject relative to the level of enzymatic activity of ENPP1 detected in a normal subject, and/or via a defect in expression of ENPP1 gene at the transcriptional (RNA and/or mRNA) or translational levels, for example via a defect in the ENPP1 gene or a control element affecting ENPP1 gene expression, relative to normal expression the ENPP1 gene in a subject. ENPP1 enzymatic activity is defined below.

“Enzymatically active” with respect to an ENPP1 polypeptide, or, as used herein, “enzymatic activity” with respect to an ENPP1 polypeptide, is defined as possessing ATP hydrolytic activity into AMP and PPi and/or AP3a hydrolysis to ATP. NPP1 readily hydrolyze ATP into AMP and PPi. The steady-state Michaelis-Menten enzymatic constants of NPP1 are determined using ATP as a substrate. NPP1 can be demonstrated to cleave ATP by HPLC analysis of the enzymatic reaction, and the identity of the substrates and products of the reaction are confirmed by using ATP, AMP, and ADP standards. The ATP substrate degrades over time in the presence of NPP1, with the accumulation of the enzymatic product AMP. Using varying concentrations of ATP substrate, the initial rate velocities for NPP1 are derived in the presence of ATP, and the data is fit to a curve to derive the enzymatic rate constants. At physiologic pH, the kinetic rate constants of NPP1 are Km=144 μM and kcat=7.8 s−1.

As used herein the term “plasma pyrophosphate (PPi) levels” refers to the amount of pyrophosphate present in plasma of animals. In certain embodiments, animals include rat, mouse, cat, dog, human, cow and horse. It is necessary to measure PPi in the plasma rather than serum because of release from platelets. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications.

Typically, plasma PPi levels in healthy human subjects range from about 1 μm to about 3 μM, in some cases between 1-2 μm. A normal level of ENPP1 in plasma refers to the amount of ENPP1 protein required to maintain a normal level of plasma pyrophosphate (PPi) in a healthy subject. A normal level of PPi in healthy humans corresponds to 2-3 μM. Subjects who have a deficiency of ENPP1 exhibit low PPi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with GACI, the PPi levels are found to be less than 1 μm and in some cases are below a detectable level. In patients afflicted with PXE, the PPi levels are below 0.5 μm. (Arterioscler Thromb Vasc Biol. 2014 September; 34(9):1985-9; Braddock et al., Nat Commun. 2015; 6: 10006.)

An “ABCC6 Deficiency” may be indicated in a number of ways, for example, by a reduced expression levels of ABCC6 enzyme in a subject. Subjects having ABCC6 deficiency can be identified by the presence of one or more physiological symptoms such as ocular calcification, skin calcification yellow papules, angioid streaks, visual impairment, calcification of soft connective tissues including the cardiovascular system, and/or, the presence of ABCC6 mutations that affect the activity and expression of ABCC6 protein. Details on diagnosis and classification of ABCC6 mutations are known in art. (Expert Opin Orphan Drugs. 2014 Jun. 1; 2(6): 567-577).

ABCC6 deficiency may result in PXE. Reduced ABCC6 enzymatic activity may occur via a reduction in the amount of ABCC6 enzyme present in a subject relative to the amount of ABCC6 enzyme present in a normal subject, and/or a reduction in the level of enzymatic activity of ABCC6 in a subject relative to the level of enzymatic activity of ABCC6 detected in a normal subject, and/or via a defect in expression of ABCC6 gene at the transcriptional (RNA and/or mRNA) or translational levels, for example via a defect in the ABCC6 gene or a control element affecting ABCC6 gene expression, relative to normal expression the ABCC6 gene in a subject.

A subject with ABCC6 deficiency can be identified by screening for ABCC6 gene mutations. Several methods for detecting gene mutations are known in art. For instance, detection of mutation in ABCC6 genes can performed by following the protocols described in J Med Genet. 2007 October; 44(10): 621-628. (Mutation detection in the ABCC6 gene and genotype-phenotype analysis in a large international case series affected by pseudoxanthoma elasticum, Ellen G Pfendner, et al)

“ABCC6 deficient patient” or “ABCC6 deficient subject” as used herein, refers to a patient having at least one pathogenic mutation in the ABCC6 gene that affects activity and/or expression of ABC66 protein.

“Pathological calcification”: As used herein, the term refers to the abnormal deposition of calcium salts in soft tissues, secretory and excretory passages of the body causing it to harden. There are two types, dystrophic calcification which occurs in dying and dead tissue and metastatic calcification which elevated extracellular levels of calcium (hypercalcemia), exceeding the homeostatic capacity of cells and tissues. Calcification can involve cells as well as extracellular matrix components such as collagen in basement membranes and elastic fibers in arterial walls. Some examples of tissues prone to calcification include: Gastric mucosa—the inner epithelial lining of the stomach, Kidneys and lungs, Cornea, Systemic arteries and Pulmonary veins.

“Pathological ossification”: As used herein, the term refers to a pathological condition in which bone arises in tissues not in the osseous system and in connective tissues usually not manifesting osteogenic properties. Ossification is classified into three types depending on the nature of the tissue or organ being affected, endochondral ossification is ossification that occurs in and replaces cartilage. Intramembranous ossification is ossification of bone that occurs in and replaces connective tissue. Metaplastic ossification the development of bony substance in normally soft body structures; called also heterotrophic ossification.

A “deficiency” of NPP1 refers to a condition in which the subject has less than or equal to 5%-10% of normal levels of NPP1 in blood plasma. Normal levels of NPP1 in healthy human subjects is approximately between 10 to 30 ng/ml. (Am J Pathol. 2001 February; 158(2): 543-554.)

A “deficiency” of ABCC6 refers to a condition in which the subject has less than or equal to 5%-10% of normal levels of ABCC6 expression. ABCC6 protein is expressed in liver and levels of ABCC6 protein can be measured by means of liver biopsy.

A “low” level of PPi refers to a condition in which the subject has less than or equal to 2%-5% of normal levels of plasma pyrophosphate (PPi). Normal levels of Plasma PPi in healthy human subjects is approximately 1.8 to 2.6 μM. (Arthritis and Rheumatism, Vol. 22, No. 8 (August 1979))

“Ectopic calcification” refers to a condition characterized by a pathologic deposition of calcium salts in tissues or bone growth in soft tissues.

“Ectopic calcification of soft tissue” refers to inappropriate biomineralization, typically composed of calcium phosphate, hydroxyapatite, calcium oxalates and octacalcium phosphates occurring in soft tissues leading to loss of hardening of soft tissues. “Arterial calcification” refers to ectopic calcification that occurs in arteries and heart valves leading to hardening and or narrowing of arteries. Calcification in arteries is correlated with atherosclerotic plaque burden and increased risk of myocardial infarction, increased ischemic episodes in peripheral vascular disease, and increased risk of dissection following angioplasty.

“Venous calcification” refers to ectopic calcification that occurs in veins that reduces the elasticity of the veins and restricts blood flow which can then lead to increase in blood pressure and coronary defects

“Vascular calcification” refers to the pathological deposition of mineral in the vascular system. It has a variety of forms, including intimal calcification and medial calcification, but can also be found in the valves of the heart. Vascular calcification is associated with atherosclerosis, diabetes, certain heredity conditions, and kidney disease, especially CKD. Patients with vascular calcification are at higher risk for adverse cardiovascular events. Vascular calcification affects a wide variety of patients. Idiopathic infantile arterial calcification is a rare form of vascular calcification where the arteries of neonates calcify.

“Brain calcification” (BC) refers to a nonspecific neuropathology wherein deposition of calcium and other mineral in blood vessel walls and tissue parenchyma occurs leading to neuronal death and gliosis. Brain calcification is” often associated with various chronic and acute brain disorders including Down's syndrome, Lewy body disease, Alzheimer's disease, Parkinson's disease, vascular dementia, brain tumors, and various endocrinologic conditions

Calcification of heart tissue refers to accumulation of deposits of calcium (possibly including other minerals) in tissues of the heart, such as aorta tissue and coronary tissue.

“Chronic kidney disease (CKD)” As used herein, the term refers to abnormalities of kidney structure or function that persist for more than three months with implications for health. Generally excretory, endocrine and metabolic functions decline together in most chronic kidney diseases. Cardiovascular disease is the most common cause of death in patients with chronic kidney disease (CKD) and vascular calcification is one of the strongest predictors of cardiovascular risk. With decreasing kidney function, the prevalence of vascular calcification increases and calcification occurs years earlier in CKD patients than in the general population. Preventing, reducing and/or reversing vascular calcification may result in increased survival in patients with CKD.

Clinical symptoms of chronic kidney diseases include itching, muscle cramps, nausea, lack of appetite, swelling of feet and ankles, sleeplessness and labored breathing. Chronic kidney disease if left untreated tends to progress into End stage renal disease (ESRD). Common symptoms of ESRD include an inability to urinate, fatigue, malaise, weight loss, bone pain, changes in skin color, a frequent formation of bruises, and edema of outer extremities like fingers, toes, hands and legs. Calciphylaxis or calcific uremic arteriopathy (CUA) is a condition that causes calcium to build up inside the blood vessels of the fat and skin. A subpopulation of patients suffering from ESRD can also develop Calciphylaxis. Common symptoms of Calciphylaxis include large purple net-like patterns on skin, deep and painful lumps that ulcerate creating open sores with black-brown crust that fails to heal, skin lesions on the lower limbs or areas with higher fat content, such as thighs, breasts, buttocks, and abdomen. A person with calciphylaxis may have higher than normal levels of calcium (hypercalcemia) and phosphate (hyperphosphatemia) in the blood. They may also have symptoms of hyperparathyroidism. Hyperparathyroidism occurs when the parathyroid glands make excess parathyroid hormone (PTH). Reduced plasma pyrophosphate (PPi) levels are also present in vascular calcification associated with end stage renal disease (ESRD).

Vascular calcifications associated with ESRD contributes to poor outcomes by increasing pulse pressure, causing or exacerbating hypertension, and inducing or intensifying myocardial infarctions and strokes. Most patients with ESRD do not die of renal failure, but from the cardiovascular complications of ESRD, and it is important to note that many very young patients with ESRD on dialysis possess coronary artery calcifications. The histologic subtype of vascular calcification associated with CKD is known as Mönckeburg's sclerosis, which is a form of vessel hardening in which calcium deposits are found in the muscular layers of the medial vascular wall. This form of calcification is histologically distinct from intimal or neo-intimal vascular wall calcification commonly observed in atherosclerosis but identical to the vascular calcifications observed in human CKD patients, and in the rodent models of the disease described herein.

“Generalized arterial calcification of infants (GACI)” (also known as IACI)”, as used herein, refers to a disorder affecting the circulatory system that becomes apparent before birth or within the first few months of life. It is characterized by abnormal accumulation of the mineral calcium (calcification) in the walls of the blood vessels that carry blood from the heart to the rest of the body (the arteries). Calcification often occurs along with thickening of the lining of the arterial walls (the intima). These changes lead to narrowing (stenosis) and stiffness of the arteries, which forces the heart to work harder to pump blood. As a result, heart failure may develop in affected individuals, with signs and symptoms including difficulty breathing, accumulation of fluid (edema) in the extremities, a bluish appearance of the skin or lips (cyanosis), severe high blood pressure (hypertension), and an enlarged heart (cardiomegaly). People with GACI may also have calcification in other organs and tissues, particularly around the joints. In addition, they may have hearing loss or softening and weakening of the bones referred to as rickets.

General arterial calcification (GACI) or Idiopathic Infantile Arterial Calcification (IIAC) characterized by abnormal accumulation of the mineral calcium (calcification) in the walls of the blood vessels that carry blood from the heart to the rest of the body (the arteries). The calcification often occurs along with thickening of the lining of the arterial walls (the intima). These changes lead to narrowing (stenosis) and stiffness of the arteries, which forces the heart to work harder to pump blood. As a result, heart failure may develop in affected individuals, with signs and symptoms including difficulty breathing, accumulation of fluid (edema) in the extremities, a bluish appearance of the skin or lips (cyanosis), severe high blood pressure (hypertension), and an enlarged heart (cardiomegaly).

The term “Infant” as used herein refers to a human child in his or her first year of life. Typically, infantile stage refers to the age during which the child is unable to walk.

“Arterial calcification” or “Vascular calcification” or “hardening of arteries”, As used herein, the term refers to a process characterized by thickening and loss of elasticity of muscular arteries walls. The thickening and loss of elasticity occurs in two distinct sites, the intimal and medial layers of the vasculatures (Medial vascular calcification). Intimal calcification is associated with atherosclerotic plaques and medial calcification is characterized by vascular stiffening and arteriosclerosis. This results in a reduction of arterial elasticity and an increased propensity for morbidity and mortality due to the impairment of the cardiovascular system's hemodynamics.

“Mineral bone disorders (MBD)”, as used herein, the term refers to a disorder characterized by abnormal hormone levels cause calcium and phosphorus levels in a person's blood to be out of balance. Mineral and bone disorder commonly occurs in people with CKD and affects most people with kidney failure receiving dialysis.

Osteopenia is a bone condition characterized by decreased bone density, which leads to bone weakening and an increased risk of bone fracture. Osteomalacia is a bone disorder characterized by decreased mineralization of newly formed bone. Osteomalacia is caused by severe vitamin D deficiency (which can be nutritional or caused by a hereditary syndrome) and by conditions that cause very low blood phosphate levels. Both osteomalacia and osteopenia increase the risk of breaking a bone. Symptoms of osteomalacia include bone pain and muscle weakness, bone tenderness, difficulty walking, and muscle spasms.

“Age related osteopenia”, as used herein refers to a condition in which bone mineral density is lower than normal. Generally, patients with osteopenia have a bone mineral density T-score of between −1.0 and −2.5. Osteopenia if left untreated progresses into Osteoporosis where bones become brittle and are extremely prone to fracture.

“Ossification of posterior longitudinal ligament (OPLL)”, as used herein, the term refers to a hyperostotic (excessive bone growth) condition that results in ectopic calcification of the posterior longitudinal ligament. The posterior longitudinal ligament connects and stabilizes the bones of the spinal column. The thickened or calcified ligament may compress the spinal cord, producing myelopathy. Symptoms of myelopathy include difficulty walking and difficulty with bowel and bladder control. OPLL may also cause radiculopathy, or compression of a nerve root. Symptoms of cervical radiculopathy include pain, tingling, or numbness in the neck, shoulder, arm, or hand.

Clinical symptoms and signs caused by OPLL are categorized as: (1) myelopathy, or a spinal cord lesion with motor and sensory disturbance of the upper and lower limbs, spasticity, and bladder dysfunction; (2) cervical radiculopathy, with pain and sensory disturbance of the upper limbs; and (3) axial discomfort, with pain and stiffness around the neck. The most common symptoms in the early stages of OPLL include dysesthesia and tingling sensation in hands, and clumsiness. With the progression of neurologic deficits, lower extremity symptoms, such as gait disturbance may appear. OPLL is detected on lateral plain radiographs, and the diagnosis and morphological details of cervical OPLL have been clearly demonstrated by magnetic resonance imaging (MRI) and computed tomography (CT).

“Pseudoxanthoma elasticum (PXE)”, as used herein, the term refers a progressive disorder that is characterized by the accumulation of deposits of calcium and other minerals (mineralization) in elastic fibers. Elastic fibers are a component of connective tissue, which provides strength and flexibility to structures throughout the body. In PXE, mineralization can affect elastic fibers in the skin, eyes, and blood vessels, and less frequently in other areas such as the digestive tract. People with PXE may have yellowish bumps called papules on their necks, underarms, and other areas of skin that touch when a joint bends. Mineralization of the blood vessels that carry blood from the heart to the rest of the body (arteries) may cause other signs and symptoms of PXE. For example, people with this condition can develop narrowing of the arteries (arteriosclerosis) or a condition called claudication that is characterized by cramping and pain during exercise due to decreased blood flow to the arms and legs.

Pseudoxanthoma elasticum (PXE), also known as Grönblad-Strandberg syndrome, is a genetic disease that causes fragmentation and mineralization of elastic fibers in some tissues. The most common problems arise in the skin and eyes, and later in blood vessels in the form of premature atherosclerosis. PXE is caused by autosomal recessive mutations in the ABCC6 gene on the short arm of chromosome 16 (16p13.1). In some cases, a portion of infants survive GACI and end up developing Pseudoxanthoma elasticum (PXE) when they grow into adults. PXE is characterized by the accumulation of calcium and other minerals (mineralization) in elastic fibers, which are a component of connective tissue. Connective tissue provides strength and flexibility to structures throughout the body. Features characteristic of PXE that also occur in GACI include yellowish bumps called papules on the underarms and other areas of skin that touch when a joint bends (flexor areas); arterial stenosis, and abnormalities called angioid streaks affecting tissue at the back of the eye (retinal hemorrhage), which is detected during an eye examination.

“End stage renal disease (ESRD), as used herein, the term refers to an advanced stage of chronic kidney disease where kidneys of the patient are no longer functional. Common symptoms include fatigue associated with anemia (low blood iron), decreased appetite, nausea, vomiting, abnormal lab values including elevated potassium, abnormalities in hormones related to bone health, elevated phosphorus and/or decreased calcium, high blood pressure (hypertension), swelling in hands/legs/eyes/lower back (sacrum) and shortness of breath.

“Calcific uremic arteriopathy (CUA)” or “Calciphylaxis”, as used herein refers to a condition with high morbidity and mortality seen in patients with kidney disease, especially in those with end stage renal disease (ESRD). It is characterized by calcification of the small blood vessels located within the fatty tissue and deeper layers of the skin leading to blood clots, and the death of skin cells due to reduced blood flow caused by excessive calcification.

“Hypophosphatemic rickets”, as used herein refers to a disorder in which the bones become soft and bend easily, due to low levels of phosphate in the blood. Symptoms usually begin in early childhood and can range in severity from bowing of the legs, bone deformities; bone pain; joint pain; poor bone growth; and short stature.

“Hereditary Hypophosphatemic Rickets” as used herein refers to a disorder related to low levels of phosphate in the blood (hypophosphatemia). Phosphate is a mineral that is essential for the normal formation of bones and teeth. Most commonly, it is caused by a mutation in the PHEX gene. Other genes that can be responsible for the condition include the CLCN5, DMP1, ENPP1, FGF23, and SLC34A3 genes. Other signs and symptoms of hereditary hypophosphatemic rickets can include premature fusion of the skull bones (craniosynostosis) and dental abnormalities. The disorder may also cause abnormal bone growth where ligaments and tendons attach to joints (enthesopathy). In adults, hypophosphatemia is characterized by a softening of the bones known as osteomalacia. Another rare type of the disorder is known as hereditary hypophosphatemic rickets with hypercalciuria (HHRH) wherein in addition to hypophosphatemia, this condition is characterized by the excretion of high levels of calcium in the urine (hypercalciuria).

“X-linked hypophosphatemia (XLH)”, as used herein, the term X-linked hypophosphatemia (XLH), also called X-linked dominant hypophosphatemic rickets, or X-linked Vitamin D-resistant rickets, is an X-linked dominant form of rickets (or osteomalacia) that differs from most cases of rickets in that vitamin D supplementation does not cure it. It can cause bone deformity including short stature and genu varum (bow leggedness). It is associated with a mutation in the PHEX gene sequence (Xp.22) and subsequent inactivity of the PHEX protein.

“Autosomal Recessive Hypophosphatemia Rickets type 2 (ARHR2)”, as used herein, the term refers to a hereditary renal phosphate-wasting disorder characterized by hypophosphatemia, rickets and/or osteomalacia and slow growth. Autosomal recessive hypophosphatemic rickets type 2 (ARHR2) is caused by homozygous loss-of-function mutation in the ENPP1 gene.

“Autosomal Dominant Hypophosphatemic Rickets (ADHR)”, as used herein refers to a rare hereditary disease in which excessive loss of phosphate in the urine leads to poorly formed bones (rickets), bone pain, and tooth abscesses. ADHR is caused by a mutation in the fibroblast growth factor 23 (FGF23). ADHR is characterized by impaired mineralization of bone, rickets and/or osteomalacia, suppressed levels of calcitriol (1,25-dihydroxyvitamin D3), renal phosphate wasting, and low serum phosphate. Mutations in FGF23 render the protein more stable and uncleavable by proteases resulting in enhanced bioactivity of FGF23. The enhanced activity of FGF23 mutants reduce expression of sodium-phosphate co-transporters, NPT2a and NPT2c, on the apical surface of proximal renal tubule cells, resulting in renal phosphate wasting.

Hypophosphatemic rickets (previously called vitamin D-resistant rickets) is a disorder in which the bones become painfully soft and bend easily, due to low levels of phosphate in the blood. Symptoms may include bowing of the legs and other bone deformities; bone pain; joint pain; poor bone growth; and short stature. In some affected babies, the space between the skull bones closes too soon leading to craniosynostosis. Most patients display Abnormality of calcium-phosphate metabolism, Abnormality of dental enamel, Delayed eruption of teeth and long, narrow head (Dolichocephaly).

“Pre-treatment”, as used herein, means treatment prior to commencement of a treatment method described herein.

“PCSK9 inhibitor” as used herein, the term refers to inhibitor that blocks the PCSK9 enzyme. Proprotein convertase subtilisin/kexin type 9 (PCSK9) is an enzyme that binds to low-density lipoprotein receptors (LDL receptors), which stops LDL being removed from the blood, leading to an increase in blood levels of LDL. The PCSK9 inhibitor blocks the PCSK9 enzyme, resulting in more LDL receptors available to remove LDL from the blood, which produces in a decrease in LDL blood levels. Commonly known PCSK9 inhibitors include but not limited to Repatha (evolocumab), Praulent (alirocumab). See “PCSK9 inhibitors: A new era of lipid lowering therapy”, Chaudhary et al., World J Cardiol. 2017 Feb. 26; 9(2): 76-91.

“Statins”, as used herein, the term refers to a class of lipid-lowering medications that reduce illness and mortality in those who are at high risk of cardiovascular disease. They lower the level of cholesterol in the blood by reducing the production of cholesterol by the liver Example of statins commonly known include but not limited to Atorvastatin (Lipitor), Lovastatin (Altoprev), Pitavastatin (Livalo, Zypitamag), Pravastatin (Pravachol), Rosuvastatin (Crestor, Ezallor), and Simvastatin (Zocor).

“Malignancy”, as used herein, refers to the presence of cancerous cells that have the ability to spread to other sites in the body (metastasize) or to invade nearby (locally) and destroy tissues. Malignant cells tend to have fast, uncontrolled growth and do not die normally due to changes in their genetic makeup. There are several main types of malignancy. Carcinoma is a malignancy that begins in the skin or in tissues that line or cover internal organs. Sarcoma is a malignancy that begins in bone, cartilage, fat, muscle, blood vessels, or other connective or supportive tissue. Leukemia is a malignancy that begins in blood-forming tissue, such as the bone marrow, and causes too many abnormal blood cells to be made. Lymphoma and multiple myeloma are malignancies that begin in the cells of the immune system. Central nervous system cancers are malignancies that begin in the tissues of the brain and spinal cord.

“Nonmelanoma Skin Cancer”, as used herein, refer to any cancer that forms in the basal, squamous or Merkel cells of the skin. Melanoma is a cancer that develops in the skin's melanocytes.

“Sponsor of study” as used herein, refers to an individual, institution, company or organization (for example, a contract research organization) that takes the responsibility to initiate, manage or finance the clinical trial but does not actually conduct the investigation.

The term “subject”, as used herein, refers to an individual, such as a mammal, such as a human, a non-human primate (e.g. chimpanzees and other apes and monkey species), a farm animal (e.g. birds, fish, cattle, sheep, pigs, goats, and horses), a domestic mammal (e.g. dogs and cats), or a laboratory animal (e.g. rodents, such as mice, rats and guinea pigs). The term includes a subject of any age or sex. In another embodiment the subject is a mammal, preferably a human.

A disease or disorder is “alleviated” if the severity of a symptom of the disease or disorder, the frequency with which such a symptom is experienced by a patient, or both, is reduced.

As used herein the terms “alteration,” “defect,” “variation” or “mutation” refer to a mutation in a gene in a cell that affects the function, activity, expression (transcription or translation) or conformation of the polypeptide it encodes, including missense and nonsense mutations, insertions, deletions, frameshifts and premature terminations.

A “disease” is a state of health of an animal wherein the animal cannot maintain homeostasis, and wherein if the disease is not ameliorated then the animal's health continues to deteriorate.

A “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “immune response” or “immune reaction” refers to the host's immune system to antigen in an invading (infecting) pathogenic organism, or to introduction or expression of foreign protein. The immune response is generally humoral and local; antibodies produced by B cells combine with antigen in an antigen-antibody complex to inactivate or neutralize antigen. Immune response is often observed when human proteins are injected into mouse model systems. Generally, the mouse model system is made immune tolerant by injecting immune suppressors prior to the introduction of a foreign antigen to ensure better viability.

As used herein, the term “immune suppression” is a deliberate reduction of the activation or efficacy of the host immune system using immune suppressant drugs to facilitate immune tolerance towards foreign antigens such as foreign proteins, organ transplants, bone marrow and tissue transplantation. Non-limiting examples of immunosuppressant drugs include anti-CD4(GK1.5) antibody, Cyclophosphamide, Azathioprine (Imuran), Mycophenolate mofetil (Cellcept), Cyclosporine (Neoral, Sandimmune, Gengraf), Methotrexate (Rheumatrex), Leflunomide (Arava), Cyclophosphamide (Cytoxan) and Chlorambucil (Leukeran).

As used herein, the term “ENPP” or “NPP” refers to ectonucleotide pyrophosphatase/phosphodiesterase.

As used herein, the term “ENPP1 protein” or “ENPP1 polypeptide” refers to ectonucleotide pyrophosphatase/phosphodiesterase-1 protein encoded by the ENPP1 gene. The encoded protein is a type II transmembrane glycoprotein and cleaves a variety of substrates, including phosphodiester bonds of nucleotides and nucleotide sugars and pyrophosphate bonds of nucleotides and nucleotide sugars. ENPP1 protein has a transmembrane domain and soluble extracellular domain. The extracellular domain is further subdivided into somatomedin B domain, catalytic domain (residues 186 to 586 of SEQ ID NO: 1) and the nuclease domain (residues 524 to 885 of SEQ ID NO: 1). The sequence and structure of wild-type ENPP1 is described in detail in PCT Application Publication No. WO 2014/126965 to Braddock, et al., which is incorporated herein in its entirety by reference.

Mammal ENPP1 polypeptides, mutants, or mutant fragments thereof, have been previously disclosed in International PCT Application Publications No. WO 2014/126965—Braddock et al., WO 2016/187408—Braddock et al., WO 2017/087936—Braddock et al., and WO2018/027024—Braddock et al., all of which are incorporated by reference in their entireties herein.

As used herein, the term “ENPP1 precursor protein” refers to ENPP1 with its signal peptide sequence at the ENPP1 N-terminus. Upon proteolysis, the signal sequence is cleaved from ENPP1 to provide the ENPP1 protein. Signal peptide sequences useful within the invention include, but are not limited to, Albumin signal sequence, Azurocidin signal sequence, ENPP1 signal peptide sequence, ENPP2 signal peptide sequence, ENPP7 signal peptide sequence, and/or ENPP5 signal peptide sequence.

As used herein, the term “ENPP1-Fc construct” refers to ENPP1 recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

As used herein, the term “Fc” refers to a human IgG (immunoglobulin) Fc domain. Subtypes of IgG such as IgG1, IgG2, IgG3, and IgG4 are contemplated for use as Fc domains.

As used herein, the “Fc region or Fc polypeptide” is the portion of an IgG molecule that correlates to a crystallizable fragment obtained by papain digestion of an IgG molecule. The Fc region comprises the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc fragment contains the entire second constant domain CH2 (residues 231-340 of human IgG1, according to the Kabat numbering system) and the third constant domain CH3 (residues 341-447). The term “IgG hinge-Fc region” or “hinge-Fc fragment” refers to a region of an IgG molecule consisting of the Fc region (residues 231-447) and a hinge region (residues 216-230) extending from the N-terminus of the Fc region. The term “constant domain” refers to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CHL domain of the light chain.

As used herein, the term “fragment,” as applied to a nucleic acid, refers to a subsequence of a larger nucleic acid. A “fragment” of a nucleic acid can be at least about 15, 50-100, 100-500, 500-1000, 1000-1500 nucleotides, 1500-2500, or 2500 nucleotides (and any integer value in between). As used herein, the term “fragment,” as applied to a protein or peptide, refers to a subsequence of a larger protein or peptide, and can be at least about 20, 50, 100, 200, 300 or 400 amino acids in length (and any integer value in between).

“Isolated” means altered or removed from the natural state. For example, a nucleic acid or a polypeptide naturally present in a living animal is not “isolated,” but the same nucleic acid or polypeptide partially or completely separated from the coexisting materials of its natural state is “isolated.” An isolated nucleic acid or protein can exist in substantially purified form, or can exist in a non-native environment such as, for example, a host cell.

As used herein, the term “patient,” “individual” or “subject” refers to a human.

As used herein, the term “pharmaceutical composition” or “composition” refers to a mixture of at least one compound useful within the invention with a pharmaceutically acceptable carrier. The pharmaceutical composition facilitates administration of the compound to a patient. Multiple techniques of administering a compound exist in the art including, but not limited to, subcutaneous, intravenous, oral, aerosol, inhalational, rectal, vaginal, transdermal, intranasal, buccal, sublingual, parenteral, intrathecal, intragastrical, ophthalmic, pulmonary, and topical administration.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained; for example, phosphate-buffered saline (PBS).

As used herein the term “plasma pyrophosphate (PPi) levels” refers to the amount of pyrophosphate present in plasma of animals. In certain embodiments, animals include rat, mouse, cat, dog, human, cow and horse. It is necessary to measure PPi in plasma rather than serum because of release from platelets. There are several ways to measure PPi, one of which is by enzymatic assay using uridine-diphosphoglucose (UDPG) pyrophosphorylase (Lust & Seegmiller, 1976, Clin. Chim. Acta 66:241-249; Cheung & Suhadolnik, 1977, Anal. Biochem. 83:61-63) with modifications. Typically, normal PPi levels in healthy subjects range from about 1 μm to about 3 μM, in some cases between 1-2 μm. Subjects who have defective ENPP1 expression tend to exhibit low PPi levels which range from at least 10% below normal levels, at least 20% below normal levels, at least 30% below normal levels, at least 40% below normal levels, at least 50% below normal levels, at least 60% below normal levels, at least 70% below normal levels, at least 80% below normal levels and combinations thereof. In patients afflicted with GACI, the PPi levels are found to be less than 1 μm and in some cases are below the level of detection. In patients afflicted with PXE, the PPi levels are below 0.5 μm. (Arterioscler Thromb Vasc Biol. 2014 September; 34(9):1985-9; Braddock et al., Nat Commun. 2015; 6: 10006.)

As used herein, the term “polypeptide” refers to a polymer composed of amino acid residues, related naturally occurring structural variants, and synthetic non-naturally occurring analogs thereof linked via peptide bonds.

As used herein, the term “PPi” refers to pyrophosphate.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

“Sample” or “biological sample” as used herein means a biological material isolated from a subject. The biological sample may contain any biological material suitable for detecting a mRNA, polypeptide or other marker of a physiologic or pathologic process in a subject, and may comprise fluid, tissue, cellular and/or non-cellular material obtained from the individual.

As used herein, “substantially purified” refers to being essentially free of other components. For example, a substantially purified polypeptide is a polypeptide that has been separated from other components with which it is normally associated in its naturally occurring state. Non-limiting embodiments include 95% purity, 99% purity, 99.5% purity, 99.9% purity and 100% purity.

As used herein, the term “treatment” or “treating” is defined as the application or administration of a therapeutic agent, i.e., a compound useful within the invention (alone or in combination with another pharmaceutical agent), to a patient, or application or administration of a therapeutic agent to an isolated tissue or cell line from a patient (e.g., for diagnosis or ex vivo applications), who has a disease or disorder, a symptom of a disease or disorder or the potential to develop a disease or disorder, with the purpose to cure, heal, alleviate, relieve, alter, remedy, ameliorate, improve or affect the disease or disorder, the symptoms of the disease or disorder, or the potential to develop the disease or disorder. Such treatments may be specifically tailored or modified, based on knowledge obtained from the field of pharmacogenomics.

The terms “prevent,” “preventing,” and “prevention”, as used herein, refer to inhibiting the inception or decreasing the occurrence of a disease in a subject. Prevention may be complete (e.g. the total absence of pathological cells in a subject) or partial. Prevention also refers to a reduced susceptibility to a clinical condition.

As used herein, the term “wild-type” refers to a gene or gene product isolated from a naturally occurring source. A wild-type gene is most frequently observed in a population and is thus arbitrarily designed the “normal” or “wild-type” form of the human NPP1 genes. In contrast, the term “functionally equivalent” refers to a NPP1 gene or gene product that displays modifications in sequence and/or functional properties (i.e., altered characteristics) when compared to the wild-type gene or gene product. Naturally occurring mutants can be isolated; these are identified by the fact that they have altered characteristics (including altered nucleic acid sequences) when compared to the wild-type gene or gene product.

The term “functional equivalent variant”, as used herein, relates to a polypeptide substantially homologous to the sequences of ENPP1 (defined above) and that preserves the enzymatic and biological activities of ENPP1. Methods for determining whether a variant preserves the biological activity of the native ENPP1 are widely known to the skilled person and include any of the assays used in the experimental part of said application. Particularly, functionally equivalent variants of ENPP1 delivered by viral vectors is encompassed by the present invention.

The functionally equivalent variants of ENPP1 are polypeptides substantially homologous to the native ENPP1. The expression “substantially homologous”, relates to a protein sequence when said protein sequence has a degree of identity with respect to the ENPP1 sequences described above of at least 80%, at least 85%, at least 90%, at least 91%, at least 92%, at least 93%, at least 94%, at least 95%, at least 96%, at least 97%, at least 98% or at least 99% respectively.

The degree of identity between two polypeptides is determined using computer algorithms and methods that are widely known for the persons skilled in the art. The identity between two amino acid sequences is preferably determined by using the BLASTP algorithm (BLAST Manual, Altschul, S., et al., NCBI NLM NIH Bethesda, Md. 20894, Altschul, S., et al., J. Mol. Biol. 215: 403-410 (1990)), though other similar algorithms can also be used. BLAST and BLAST 2.0 are used, with the parameters described herein, to determine percent sequence identity. Software for performing BLAST analyses is publicly available through the National Center for Biotechnology Information.

“Functionally equivalent variants” of ENPP1 may be obtained by replacing nucleotides within the polynucleotide accounting for codon preference in the host cell that is to be used to produce the ENPP1 respectively. Such “codon optimization” can be determined via computer algorithms which incorporate codon frequency tables such as “Human high.cod” for codon preference as provided by the University of Wisconsin Package Version 9.0, Genetics Computer Group, Madison, Wis.

“About” as used herein when referring to a measurable value such as an amount, a temporal duration, and the like, is meant to encompass variations of 10% or ±5%, in certain embodiments ±1-5%, in certain embodiments ±5%, in certain embodiments ±4%, in certain embodiments ±4%, in certain embodiments ±3%, in certain embodiments ±2%, and in certain embodiments ±1% from the specified value (0.2 mg/kg or 0.6 mg/kg or 1.8 mg/kg), as such variations are appropriate to perform the disclosed methods.

The disclosure provides a representative example of protein sequences. The protein sequences described can be converted into nucleic acid sequences by performing revere translation and codon optimization. There are several tools available in art such as Expasy (https://www.expasy.org/) and bioinformatics servers (http://www.bioinformatics.org) that enable such conversions

Ranges: throughout this disclosure, various aspects according to the invention can be presented in a range format. It should be understood that the description in range format is merely for convenience and brevity and should not be construed as an inflexible limitation on the scope according to the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

### ABCC6 Mutation Detection

Genomic DNA is isolated from peripheral blood samples of subjects suspected of having ABC66 deficiency using commercially available DNA isolation kits. (Puregene DNA Isolation Kit; Gentra Systems, Minneapolis, Minnesota, USA). Control genomic DNA is obtained from the human lymphoblastoid cell line K562 (American Type Culture Collection, Manassas, Virginia, USA). All DNA samples are adjusted with water to a concentration of 10 ng/μL.

The mutation-detection strategy is based on: (1) identification of the recurrent mutations R1141X and del23-29 by restriction-enzyme digestion; (2) optimised denaturing high-performance liquid chromatography (dHPLC) scanning of PCR products corresponding to all exons in subjects in whom the two recurrent mutations are not identified on both alleles, followed by (3) sequencing of exons with altered dHPLC patterns; and (4) confirmation of novel mutations by restriction-enzyme digestion or resequencing.

Screening for the recurrent mutations R1141X and del23-29 is performed as previously described. (Compound heterozygosity for a recurrent 16.5-kb Alu-mediated deletion mutation and single-base-pair substitutions in the ABCC6 gene results in pseudoxanthoma elasticum. Ringpfeil F, Nakano A, Uitto J, Pulkkinen L, Am J Hum Genet. 2001 March; 68(3):642-52.) Conditions and primers for generating PCR products spanning all exons of the coding regions and flanking intronic sequences of the ABCC6 gene are identified for optimum dHPLC screening in supplementary table (J Med Genet. 2007 October; 44(10): 621-628).

These primers are designed to exclude the pseudogenes homologous to exons 1-4 and 1-931 and to anneal within ˜50 bases of the 5′ and 3′ ends of the exon and to exclude known intronic polymorphisms where possible. PCR for dHPLC analysis is performed using 1.5 U Taq polymerase (Qiagen Inc., Valencia, California, USA) mixed with 5 U Optimase Taq polymerase (Transgenomic, Gaithersburg, Maryland, USA) and Q buffer (Qiagen), according to the manufacturer' instructions. PCR reactions contained 200 ng DNA as template and 20 ng of each primer in a final volume of 50 μl. Cycling conditions for all primer pairs were 94° C. for 5 min, followed by 41 cycles of 94° C. for 1 min, annealing temperature for a particular primer pair (range 55-60° C.) for 1 min and 72° C. for 1 min, with a final step at 72° C. for 5 min.

The PCR products generated using patients with PXE DNA as template are allowed to form heteroduplexes with an equal volume of a PCR product of the same exon amplified from template DNA of the lymphoblastoid control cell line K562. For this purpose, the PCR products were mixed in a 1:1 ratio and denatured at 94° C. for 10 min, followed by reannealing at 65° C. for 15 min and 37° C. for 15 min. The PCR products are then screened by dHPLC (WAVE; Transgenomic, Gaithersburg, Maryland, USA) using methods designed to enhance partial denaturation of the PCR products containing mismatched bases (see supplementary table 1 of J Med Genet. 2007 October; 44(10): 621-628). PCR products showing pattern shifts are sequenced in both directions in most cases. DNA sequencing is performed on an automated sequencer (ABI Prism 377 or ABI 3100; Perkin-Elmer-Cetus, Foster City, California, USA). Putative mutations are confirmed by restriction-enzyme digestion followed by agarose-gel electrophoresis or by resequencing of a new PCR product when a suitable restriction enzyme was not available. The subjects are then identified as subjects with ABCC6 mutations if they were found to contain known mutations in ABCC6 genes that affect the activity and or expression level of ABCC6 protein.

### DETAILED DESCRIPTION

### 1. Overview

The disclosure provides for treating, preventing, or reducing the progression rate and/or severity of pathologic calcification and/or ossification or one or more complications of pathologic calcification and/or ossification by administering to a subject ENPP1-FC subcutaneously (SC) at a dose of about 0.2 mg/kg, about 0.6 mg/kg, or about 1.8 mg/kg. With respect to a recited dose of ENPP1-FC, “about” mean a degree of error given the nature or precision of the measurements within ±5 percent (%), ±4%, ±3%, or ±2% or ±1% of the recited dose in mg ENPP1-FC/kg body weight of the subject.

### 2. Administered Dose of ENPP1 Agent

An ENPP1 agent is administered at a dose of about 0.2 mg/kg, about 0.6 mg/kg, or about 1.8 mg/kg. A medical practitioner will select which of these doses is administered to a given subject, and may be guided by a subject's serum PPi levels, the selected dose being sufficient to restore PPi to normal levels in the subject.

### 3. ENPP1 Agent

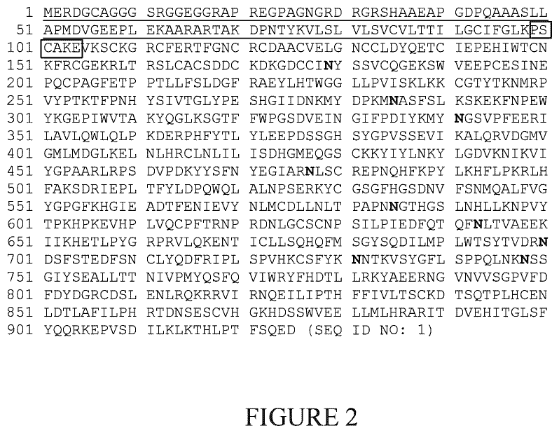

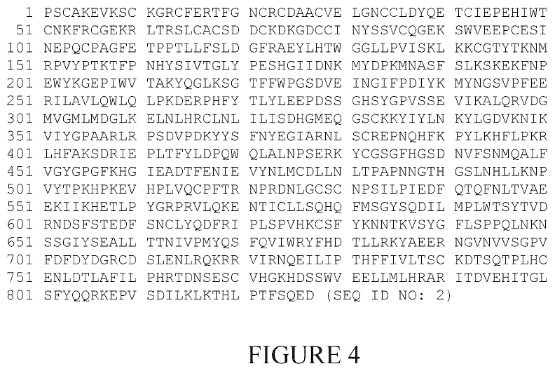

An ENPP1 agent is an ENPP1 polypeptide. ENPP1 polypeptides disclosed herein include naturally occurring polypeptides of the ENPP1 family as well as any variants thereof (including mutants, fragments, fusions, and peptidomimetic forms) that retain a biological activity. The terms “ENPP1” or “ENPP1 polypeptide” refers to ectonucleotide pyrophosphatase/phosphodiesterase 1 proteins (NPP1/ENPP1/PC-1) and ENPP1-related proteins, derived from any species. ENPP1 protein comprises a type II transmembrane glycoprotein that forms a homodimer. Each monomer of the ENPP1 protein comprises a short intracellular N-terminal domain involved in targeting to the plasma membrane, a transmembrane domain, and a large extracellular region comprising several domains. The large extracellular region comprises SMB1 and SMB2 domains, which have been reported to take part in ENPP1 dimerization (R. Gijsbers, H. et al., Biochem. J. 371; 2003: 321-330). Specifically, the SMB domains contain eight cysteine residues, each arranged in four disulphide bonds, and have been shown to mediate ENPP1 homodimerization through covalent cystine inter- and intramolecular bonds. The protein cleaves a variety of substrates, including phosphodiester bonds of nucleotides and nucleotide sugars and pyrophosphate bonds of nucleotides and nucleotide sugars. ENPP1 protein functions to hydrolyze nucleoside 5′ triphosphatase to either corresponding monophosphates and also hydrolyzes diadenosine polyphosphates. ENPP1 proteins play a role in purinergic signaling which is involved in the regulation of cardiovascular, neurological, immunological, musculoskeletal, hormonal, and hematological functions. An exemplary amino acid sequence of the human ENPP1 precursor protein (NCBI accession NP_006199) is shown in FIG. 2 (SEQ ID NO: 1). The human ENPP1 precursor protein includes an endogenous ENPP1 signal peptide sequence at the ENPP1 N-terminus. Numbering of amino acids for all ENPP1-related polypeptides described herein is based on the numbering of the human ENPP1 precursor protein sequence provided in FIG. 2 unless specifically designated otherwise. In certain embodiments, the ENPP1 precursor protein further comprises an endogenous or heterologous signal peptide sequence. Upon proteolysis, the signal peptide sequence is cleaved from the ENPP1 precursor protein to provide the mature ENPP1 protein. See, e.g., Jansen S, et al. J Cell Sci. 2005; 118(Pt 14):3081-9. Exemplary signal peptide sequences that can be used with the polypeptides disclosed herein include, but are not limited to, ENPP1 signal peptide sequence, ENPP2 signal peptide sequence, ENPP7 signal peptide sequence, and/or ENPP5 signal peptide sequence. The processed (mature) extracellular ENPP1 polypeptide sequence is shown in FIG. 4 (SEQ ID NO: 2).

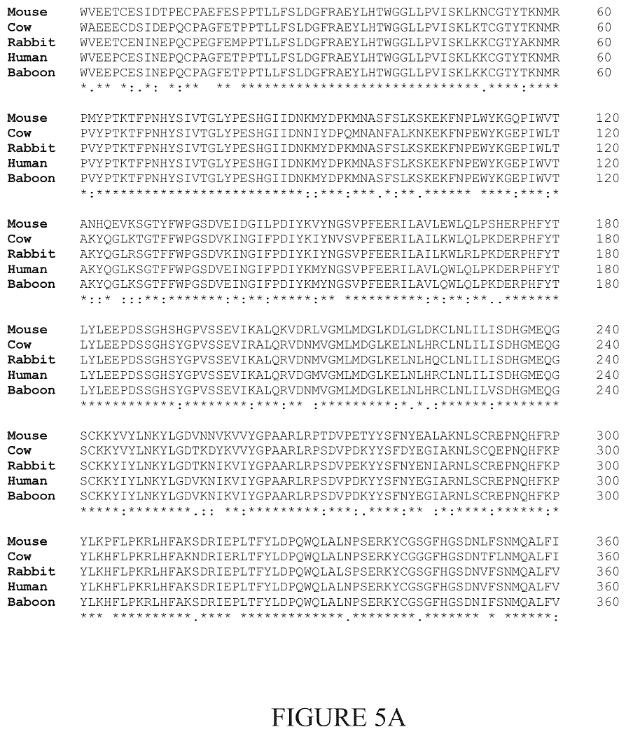

It is generally known in the art that ENPP1 is well-conserved among vertebrates, with large stretches of the extracellular domain substantially conserved. For example, FIG. 5A and FIG. 5B depict a multi-sequence alignment of a human ENPP1 extracellular domain compared to various ENPP1 orthologs. ENPP1 binding to various nucleotide triphosphates (e.g., ATP, UTP, GTP, TTP, and CTP), pNP-TMP, 3′,5′-cAMP, and 2′-3′-cGAMP is also highly conserved (see, e.g., Kato K. et al., Proc Natl Acad Sci USA. 2012; 109(42):16876-81 and Mackenzie N C, et al. Bone. 2012; 51(5):961-8). Accordingly, from these alignments, it is possible to predict key amino acid positions with the extracellular domain that are important for normal ENPP1 activities as well as to predict amino acid positions that are likely to be tolerant to substitution without significantly altering normal ENPP1 activities. Therefore, an enzymatically active, human ENPP1 polypeptide useful in accordance with the presently disclosed compositions, may include one or more amino acids at corresponding positions from the sequence of another vertebrate ENPP1, or may include a residue that is similar to that in the human or other vertebrate sequences. Substitutions of one or more amino acids at corresponding positions may include conservative variations or substitutions that are not likely to change the shape of the polypeptide chain or alter normal ENPP1 activities. Examples of conservative variations, or substitutions, include the replacement of one hydrophobic residue such as isoleucine, valine, leucine or methionine for another, or the substitution of one polar residue for another, such as the substitution of arginine for lysine, glutamic for aspartic acid, or glutamine for asparagine. For example, ENPP1 polypeptides include polypeptides derived from the sequence of any known ENPP1 polypeptide having a sequence at least about 80% identical to the sequence of an ENPP1 polypeptide, and preferably at least 85%, 90%, 91%, 92%, 93%, 94%, 95%, 96%, 97%, 98%, 99% or greater identity.

### 4. Enzymatic Activity of ENPP1

ENPP1 proteins have been characterized in the art in terms of structural and biological characteristics. In certain embodiments, soluble ENPP1 proteins disclosed herein comprise pyrophosphatase and/or phosphodiesterase activity. For instance, in some embodiments, the ENPP1 protein binds nucleotide triphosphates (e.g., ATP, UTP, GTP, TTP, and CTP), pNP-TMP, 3′,5′-cAMP, and 2′-3′-cGAMP; and converts nucleotide triphosphates into inorganic pyrophosphate [see, e.g., Kato K. et al., Proc Natl Acad Sci USA. 2012; 109(42):16876-81; Li L, et al. Nat Chem Biol. 2014; 10(12):1043-8; Jansen S, et al. Structure. 2012; 20(11):1948-59; and Onyedibe K I, et al. Molecules. 2019; 24(22)].

“Enzymatically active” or “Biologically active” ENPP1 polypeptides exhibit pyrophosphatase and/or phosphodiesterase activity (e.g., is capable of binding and/or hydrolyzing ATP into AMP and PPi and/or AP3a into ATP). For example, the pyrophosphatase/phosphodiesterase domain of an ENPP1 protein hydrolyzes extracellular nucleotide triphosphates to produce inorganic pyrophosphates (PPi) and is generally soluble. This activity can be measured using a pNP-TMP assay as previously described (Saunders, et al., 2008, Mol. Cancer Ther. 7(10):3352-62; Albright, et al., 2015, Nat Comm. 6:10006). In certain embodiments, the soluble ENPP1 polypeptide has a k_{cat} value for the substrate ATP greater than or equal to about 3.4 (±0.4) s′¹ enzyme′¹, wherein the k_{cat} is determined by measuring the rate of hydrolysis of ATP for the polypeptide. In certain embodiments, the soluble ENPP1 polypeptide has a K_{M} value for the substrate ATP less than or equal to about 2 μM, wherein the K_{M} is determined by measuring the rate of hydrolysis of ATP for the polypeptide. In addition to the teachings herein, these references provide ample guidance for how to generate soluble ENPP1 proteins that retain one or more biological activities (e.g., conversion of nucleotides into inorganic pyrophosphate).

### 5. Soluble ENPP1

In one embodiment, the disclosure relates to ENPP1 polypeptides. As described herein, the term soluble ENPP1 polypeptide, includes any naturally occurring extracellular domain of an ENPP1 protein as well as any variants thereof (including mutants, fragments and peptidomimetic forms) that retain a biological activity (e.g., enzymatically active). Examples of soluble ENPP1 polypeptides include, for example, an ENPP1 extracellular domain (SEQ ID NO: 2) as shown in FIG. 4. In certain embodiments, the soluble ENPP1 polypeptides further comprise a signal sequence in addition to the extracellular domain of an ENPP1 polypeptide. Exemplary signal sequences include the native signal sequence of an ENPP1 polypeptide, or a signal sequence from another protein, such as a hENPP7 signal sequence. Examples of variant soluble ENPP1 polypeptides are provided in International Patent Application Publication Nos. WO 2012/125182, WO 2014/126965, WO 2016/187408, WO 2018/027024, WO 2020206302 and WO 2020/047520 the contents of all of which are incorporated herein by reference in their entirety.

### 6. ENPP1 Fusion Proteins

In some embodiments, the ENPP1 polypeptide is a fusion protein comprising an ENPP1 polypeptide domain and one or more heterologous protein portions (i.e., polypeptide domains heterologous to ENPP1). An amino acid sequence is understood to be heterologous to ENPP1 if it is not uniquely found in the form of ENPP1 represented by SEQ ID NO: 1. In some embodiments, the heterologous protein portion comprises an Fc domain of an immunoglobulin. In some embodiments, the Fc domain of the immunoglobulin is an Fc domain of an IgG1 immunoglobulin. In certain embodiments, the soluble ENPP1 polypeptide is C-terminally fused to the Fc domain of human immunoglobulin 1 (IgGl), human immunoglobulin 2 (IgG2), human immunoglobulin 3 (IgG3), and/or human immunoglobulin 4 (IgG4). In other embodiments, the soluble ENPP1 polypeptide is N-terminally fused to the Fc domain of human immunoglobulin 1 (IgGl), human immunoglobulin 2 (IgG2), human immunoglobulin 3 (IgG3), and/or human immunoglobulin 4 (IgG4). In some embodiments, the presence of an Fc domain improves half-life, solubility, reduces immunogenicity, and increases the activity of the soluble ENPP1 polypeptide. In certain embodiments, portions of the native human IgG proteins (IgG1, IgG2, IgG3, and IgG4), may be used for the Fc portion (e.g., ENPP1-Fc). For instance, the present disclosure provides fusion proteins comprising ENPP1 fused to a polypeptide comprising a constant domain of an immunoglobulin, such as a CH1, CH2, or CH3 domain derived from human IgG1, IgG2, IgG3, and/or IgG4. The Fc fragment may comprise regions of the native IgG such as the hinge region (residues 216-230 of human IgG1, according to the Rabat numbering system), the entire second constant domain CH2 (residues 231-340), and the third constant domain CH3 (residues 341-447). As used herein, the term “ENPP1-Fc construct” refers to a soluble form of ENPP1 (e.g., the extracellular domain of an ENPP1 polypeptide) recombinantly fused and/or chemically conjugated (including both covalent and non-covalent conjugations) to an FcR binding domain of an IgG molecule (preferably, a human IgG). In certain embodiments, the C-terminus of ENPP1 is fused or conjugated to the N-terminus of the FcR binding domain.

An example of an amino acid sequence that may be used for the Fc portion of human IgG1 (G1Fc) is SEQ ID NO: 6 (Table 1). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 6.

In some embodiments, the heterologous protein portion comprises one or more domains selected from the group consisting of polyhistidine, FLAG tag, Glu-Glu, glutathione S-transferase (GST), thioredoxin, protein A, protein G, an immunoglobulin heavy-chain constant region (Fc), maltose binding protein (MBP), or human serum albumin. A fusion domain may be selected so as to confer a desired property. For example, some fusion domains are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a fusion domain may be selected so as to facilitate detection of the ENPP1 polypeptide. Examples of such detection domains include the various fluorescent proteins (e.g., GFP) as well as “epitope tags,” which are usually short peptide sequences for which a specific antibody is available. Well-known epitope tags for which specific monoclonal antibodies are readily available include FLAG, influenza virus haemagluttinin (HA), and c-myc tags. In some cases, the fusion domains have a protease cleavage site, such as for Factor Xa or thrombin, which allows the relevant protease to partially digest the fusion proteins and thereby liberate the recombinant proteins therefrom. The liberated proteins can then be isolated from the fusion domain by subsequent chromatographic separation.

### 7. Linkers

In some embodiments, the ENPP1 fusion protein further comprises a linker positioned between the ENPP1 polypeptide domain and the one or more heterologous protein portions (e.g., an Fc immunoglobulin domain). In certain embodiments, the soluble ENPP1 polypeptide is directly or indirectly fused to the Fc domain. In some embodiments, the soluble ENPP1 fusion protein comprises a linker between the Fc domain and the ENPP1 polypeptide. In some embodiments, a linker can be an amino acid spacer including 1-200 amino acids. Suitable peptide spacers are known in the art, and include, for example, peptide linkers containing flexible amino acid residues such as glycine, alanine, and serine. In some embodiments, the linker comprises a polyglycine linker or a Gly-Ser linker. In some embodiments, a spacer can contain motifs, e.g., multiple or repeating motifs, of GA (SEQ ID NO: 21), GS (SEQ ID NO: 22), GG (SEQ ID NO: 23), GGA (SEQ ID NO: 24), GGS (SEQ ID NO: 25), GGG (SEQ ID NO: 26), GGGA (SEQ ID NO: 27), GGGS (SEQ ID NO: 28), GGGG (SEQ ID NO: 29), GGGGA (SEQ ID NO: 30), GGGGS (SEQ ID NO: 31), GGGGG (SEQ ID NO: 32), GGAG (SEQ ID NO: 33), GGSG (SEQ ID NO: 34), AGGG (SEQ ID NO: 35), SGGGG (SEQ ID NO: 36), or SGGG (SEQ ID NO: 37). In some embodiments, a spacer can contain 2 to 12 amino acids including motifs of GA or GS, e.g., GA, GS, GAGA (SEQ ID NO: 38), GSGS (SEQ ID NO: 39), GAGAGA (SEQ ID NO: 40), GSGSGS (SEQ ID NO: 41), GAGAGAGA (SEQ ID NO: 42), GSGSGSGS (SEQ ID NO: 43), GAGAGAGAGA (SEQ ID NO: 44), GSGSGSGSGS (SEQ ID NO: 45), GAGAGAGAGAGA (SEQ ID NO: 46), and GSGSGSGSGSGS (SEQ ID NO: 47). In some embodiments, a spacer can contain 3 to 12 amino acids including motifs of GGA or GGS, e.g., GGA, GGS, GGAGGA (SEQ ID NO: 48), GGSGGS (SEQ ID NO: 49), GGAGGAGGA (SEQ ID NO: 50), GGSGGSGGS (SEQ ID NO: 51), GGAGGAGGAGGA (SEQ ID NO: 52), and GGSGGSGGSGGS (SEQ ID NO: 53). In yet some embodiments, a spacer can contain 4 to 12 amino acids including motifs of GGAG (SEQ ID NO: 54), GGSG (SEQ ID NO: 55), e.g., GGAG (SEQ ID NO: 56), GGSG (SEQ ID NO: 57), GGAGGGAG (SEQ ID NO: 58), GGSGGGSG (SEQ ID NO: 59), GGAGGGAGGGAG (SEQ ID NO: 60), and GGSGGGSGGGSG (SEQ ID NO: 61). In some embodiments, a spacer can contain motifs of GGGGA (SEQ ID NO: 62) or GGGGS (SEQ ID NO: 63), e.g., GGGGAGGGGAGGGGA (SEQ ID NO: 64) and GGGGSGGGGSGGGGS (SEQ ID NO: 65). In some embodiments of the invention, an amino acid spacer between a heterologous protein portion (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a soluble ENPP1 polypeptide may be GGG, GGGA (SEQ ID NO: 27), GGGG (SEQ ID NO: 29), GGGAG (SEQ ID NO: 66), GGGAGG (SEQ ID NO: 67), or GGGAGGG (SEQ ID NO: 68).

In some embodiments, a spacer can also contain amino acids other than glycine, alanine, and serine, e.g., LIN (SEQ ID NO: 69), TGGGG (SEQ ID NO: 70), AAAL (SEQ ID NO: 71), AAAK (SEQ ID NO: 72), AAAR (SEQ ID NO: 73), EGKSSGSGSESKST (SEQ ID NO: 74), GSAGSAAGSGEF (SEQ ID NO: 75), AEAAAKEAAAKA (SEQ ID NO: 76), KESGSVSSEQLAQFRSLD (SEQ ID NO: 77), GENLYFQSGG (SEQ ID NO: 78), SACYCELS (SEQ ID NO: 79), RSIAT (SEQ ID NO: 80), RPACKIPNDLKQKVMNH (SEQ ID NO: 81), GGSAGGSGSGSSGGSSGASGTGTAGGTGSGSGTGSG (SEQ ID NO: 82), AAANSSIDLISVPVDSR (SEQ ID NO: 83), GGSGGGSEGGGSEGGGSEGGGSEGGGSEGGGSGGGS (SEQ ID NO: 84), NSS (SEQ ID NO: 87), ESS (SEQ ID NO: 88), RQQ (SEQ ID NO: 89), KR (SEQ ID NO: 90), (R)ₘ; m=0-15 (SEQ ID NO: 91), DSSSEEKFLRRIGRFG (SEQ ID NO: 92), EEEEEEEPRGDT (SEQ ID NO: 93), APWHLSSQYSRT (SEQ ID NO: 94), STLPIPHEFSRE (SEQ ID NO: 95), VTKHLNQISQSY (SEQ ID NO: 96), (E)ₘ; m=1-15 (SEQ ID NO: 97), RSGSGGS (SEQ ID NO: 98), (D)ₘ; m=1-15 (SEQ ID NO: 99), LVIMSLGLGLGLGLRK (SEQ ID NO: 100), VIMSLGLGLGLGLRK (SEQ ID NO: 101), IMSLGLGLGLGLRK (SEQ ID NO: 102), MSLGLGLGLGLRK (SEQ ID NO: 103), SLGLGLGLGLRK (SEQ ID NO: 104), LGLGLGLGLRK (SEQ ID NO: 105), GLGLGLGLRK (SEQ ID NO: 106), LGLGLGLRK (SEQ ID NO: 107), GLGLGLRK (SEQ ID NO: 108), LGLGLRK (SEQ ID NO: 109), GLGLRK (SEQ ID NO: 110), LGLRK (SEQ ID NO: 111), GLRK (SEQ ID NO: 112), LRK (SEQ ID NO: 113), RK (SEQ ID NO: 114), or (K)ₘ; m=1-15 (SEQ ID NO: 115). In some embodiments, a spacer can contain motifs, e.g., multiple or repeating motifs, of EAAAK (SEQ ID NO: 85). In some embodiments, a spacer can contain motifs, e.g., multiple or repeating motifs, of praline-rich sequences such as (XP)n, in which X may be any amino acid (e.g., A, K, or E) and n is from 1-5, and PAPAP (SEQ ID NO: 86).

The length of the peptide spacer and the amino acids used can be adjusted depending on the two proteins involved and the degree of flexibility desired in the final protein fusion polypeptide. The length of the spacer can be adjusted to ensure proper protein folding and avoid aggregate formation.

In some embodiments, different elements of the fusion proteins (e.g., immunoglobulin Fc fusion proteins) may be arranged in any manner that is consistent with desired functionality. For example, a soluble ENPP1 polypeptide domain may be placed C-terminal to a heterologous protein portion, or alternatively, a heterologous protein portion may be placed C-terminal to a soluble ENPP1 polypeptide domain. The soluble ENPP1 polypeptide domain and the heterologous protein portion may be directly or indirectly linked in a fusion protein, and additional domains or amino acid sequences may be included C- or N-terminal to either domain or between the domains. Preferred fusion proteins comprise the amino acid sequence set forth in any one of SEQ ID NOs: 3-5.

In some embodiments, soluble ENPP1 polypeptides of the present disclosure contain one or more heterologous moieties. Optionally, a soluble ENPP1 polypeptide includes one or more heterologous moieties selected from: a glycosylated amino acid, a PEGylated amino acid, a farnesylated amino acid, an acetylated amino acid, a biotinylated amino acid, an amino acid conjugated to a lipid moiety, and an amino acid conjugated to an organic derivatizing agent. In some embodiments, a soluble ENPP1 polypeptide disclosed herein is further modified. Such modifications include, but are not limited to, acetylation, carboxylation, glycosylation, phosphorylation, lipidation, and acylation. As a result, the soluble ENPP1 polypeptide may contain non-amino acid elements, such as polyethylene glycols, lipids, polysaccharide or monosaccharide, and phosphates. Effects of such non-amino acid elements on the functionality of a soluble ENPP1 polypeptide may be tested as described herein for other soluble ENPP1 polypeptides. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the soluble ENPP1 polypeptides.

As used herein, percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, or CLUSTAL OMEGA software. In some embodiments, alignment is performed using the CLUSTAL OMEGA software. Those skilled in the art can determine appropriate parameters for aligning sequences, including any algorithms needed to achieve maximal alignment over the full length of the sequences being compared.

### 8. Determining Solubility

In some embodiments, the activity of soluble ENPP1 polypeptides may also be tested in a cell-based or in vivo assay. For example, the effect of a soluble ENPP1 polypeptide on the production of inorganic pyrophosphates (PPi) can be measured. Specifically, the pyrophosphatase/phosphodiesterase domain of an ENPP1 protein hydrolyzes extracellular nucleotide triphosphates to produce inorganic pyrophosphates (PPi) and is generally soluble. This activity can be measured using a pNP-TMP assay as well as an HPLC-based ATP hydrolysis assay, as previously described (Saunders, et al., 2008, Mol. Cancer Ther. 7(10):3352-62; Albright, et al., 2015, Nat Comm. 6:10006). The effect of soluble ENPP1 polypeptides on the expression of genes involved in ENPP1 associated diseases such as ARHR2 (e.g., transcription of fibroblast growth factor 23 in osteoblasts and osteoclasts) can be assessed. This may, as needed, be performed in the presence of one or more nucleotide triphosphates or other ENPP1 substrates, and cells may be transfected so as to produce a soluble ENPP1 polypeptide. Likewise, a soluble ENPP1 polypeptide may be administered to a mouse or other animal and effects on ENPP1 associated diseases may be assessed using art-recognized methods.

In some embodiments, ENPP1 polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC) analyses. Methods for assessment of purity are well known in the art [see, e.g., Flatman et al., (2007) J. Chromatogr. B 848:79-87]. In some embodiments, soluble ENPP1 polypeptides to be used in accordance with the methods described herein are recombinant polypeptides.

### 9. ENPP1 Production

ENPP1 polypeptides of the disclosure can be produced by a variety of art-known techniques. For example, polypeptides of the disclosure can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the polypeptides of the disclosure, including fragments or variants thereof, may be recombinantly produced using various expression systems [e.g., E. coli, Chinese Hamster Ovary (CHO) cells, COS cells, baculovirus, Yeast Pichia] as is well known in the art. The protein can be produced in either adherent or suspension cells. In some embodiments, the fusion protein is expressed in CHO cells. To establish stable cell lines the nucleic acid sequence encoding ENPP1 constructs are cloned into an appropriate vector for large scale protein production. In a further embodiment, the modified or unmodified polypeptides of the disclosure may be produced by digestion of recombinantly produced full-length ENPP1 polypeptides by using, for example, a protease, e.g., trypsin, thermolysin, chymotrypsin, pepsin, or paired basic amino acid converting enzyme (PACE). Computer analysis (using commercially available software, e.g., MacVector, Omega, PCGene, Molecular Simulation, Inc.) can be used to identify proteolytic cleavage sites. Alternatively, such polypeptides may be produced from recombinantly generated full-length ENPP1 polypeptides using chemical cleavage (e.g., cyanogen bromide, hydroxylamine, etc.).

### 10. Expression Systems

Many expression systems are known and can be used for the production of ENPP1 fusion protein, including bacteria (for example E. coli and Bacillus subtilis), yeasts (for example Saccharomyces cerevisiae, Kluyveronmyces lactis and Pichia pastoris), filamentous fungi (for example Aspergillus), plant cells, animal cells and insect cells. The desired protein can be produced in conventional ways, for example from a coding sequence inserted in the host chromosome or on a free plasmid.

The yeasts can be transformed with a coding sequence for the desired protein in any of the usual ways (e.g., electroporation). Methods for transformation of yeast by electroporation are disclosed in Becker & Guarente, 1990, Methods Enzymol. 194: 182. Successfully transformed cells, i.e., cells that contain a DNA construct of the present disclosure, can be identified by well-known techniques. For example, cells resulting from the introduction of an expression construct can be grown to produce an ENPP1 polypeptide. Cells can be harvested and lysed and their DNA content examined for the presence of the DNA using a method, such as that described by Southern, 1975, J. Mol. Biol, 98:503 and/or Berent, et al., 1985, Biotech 3:208. Alternatively, the presence of the protein in the supernatant can be detected using antibodies.

Useful yeast plasmid vectors include pRS403-406 and pRS413-416 and are generally available front Stratagene Cloning Systems, La Jolla, CA, USA Plasmids pRS403, pRS404, pRS405 and pRS406 are Yeast Integrating plasmids (Yips) and incorporate the yeast selectable markers I-11S3, TRP1, LEU2 and 1JRA3. Plasmids pRS413-416 are Yeast Centromere plasmids (YCps).

A variety of methods have been developed to operably link DNA to vectors via complementary cohesive termini. For instance, complementary homopolymer tract can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 3′-single-stranded termini with their 3′-5′-exonucleolytic activities, and fill in recessed 3′-ends with their polymerizing activities.

The combination of these activities thus generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. As a result, the products of the reaction are DNA segments carrying polymeric linker sequences at their ends. These DNA segments can be cleaved with an appropriate restriction enzyme and ligated to an expression vector that has been cleaved with an enzyme that produces termini compatible with those of the DNA segment.

Clones of single, stably transfected cells are then established and screened for high expressing clones of the desired ENPP1 fusion protein. Screening of the single cell clones for ENPP1 protein expression can be accomplished in a high-throughput manner in 96 well plates using the synthetic enzymatic substrate pNP-TMP as previously described (Albright, et al., 2015, Nat. Commun. 6:10006). Upon identification of high expressing clones through screening, protein production can be accomplished in shaking flasks or bio-reactors are previously described in Albright, et al., 2015, Nat. Commun. 6:10006.

### 11. ENPP1 Purification

Purification of ENPP1 can be accomplished using a combination of standard purification techniques known in the art. Following purification, ENPP1-Fc can be dialyzed into PBS supplemented with Zn2+ and Mg2+ (PBSplus) concentrated to between 5 and 7 mg/ml, and frozen at −80° C. in aliquots of 200-500 pl. Aliquots can be thawed immediately prior to use and the specific activity of the solution can be adjusted to 31.25 au/ml (or about 0.7 mg/ml depending on the preparation) by dilution in PBSplus.

### 12. Route and Frequency of Administration

The polypeptide may be administered acutely or chronically to the subject. In certain embodiments, a second dosage of a soluble ENPP1 polypeptide or ENPP1 fusion polypeptide disclosed herein is administered after a suitable time interval of about after two days, after four days, after a week, or after a month to the subject or even less frequently, such as once every several months or even once a year or less. The frequency of the dose is readily apparent to the skilled artisan and depends upon any number of factors, such as, but not limited to, the type and severity of the disease being treated, and the type and age of the patient.

A dose amount or frequency may be selected so that the steady state level of plasma PPi is maintained at a constant or steady state level, and/or so as to achieve a continuous level of plasma PPi that is either close to the normal (2-3 μM) level or above (30-50% higher than) normal levels of PPi and does not return to the lower level of PPi that the subject had prior to the administration of first dosage of constructs disclosed herein.

Alternative, the ENPP1 agent may be administered at appropriate time intervals of either every 2 days, or every 4 days, every week or bi weekly or once every three weeks or once every month so as to achieve a constant level of enzymatic activity of ENPP1.

Alternatively, an ENPP1 agent according to the disclosure is administered at an appropriate time interval of every 2 days, or every 4 days, or every week or bi weekly or once every three weeks or once every month by monitoring one or more symptoms of a subject's disease or disorder.

Without wishing to be bound by theory, it is believed that maintaining a steady state concentration of plasma PPi at normal levels reduces and/or prevents progression of pathological calcification of subjects.

In certain embodiments, the polypeptide is administered locally, regionally, parenterally or systemically to the subject. In some embodiments, the polypeptide is administered subcutaneously.

As used herein, “parenteral administration” of a formulation includes any route of administration characterized by physical breaching of a tissue of a subject and administration of the ENPP1 agent through the breach in the tissue. Parenteral administration thus includes, but is not limited to, administration of an ENPP1 agent by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrastemal injection, and kidney dialytic infusion techniques.

The regimen of administration may affect what constitutes an effective amount. For example, several divided dosages, as well as staggered dosages may be administered in a given time period (daily) or sequentially, or the dose may be continuously infused, or may be a bolus injection. Further, selection of a recited dose of an ENPP1 agent may be indicated by the exigencies of the therapeutic or prophylactic situation.

Administration of the compositions of the present disclosure (e.g., soluble ENPP1 polypeptides and fusion proteins thereof) to a patient, such as a mammal (i.e., a human), may be carried out using known procedures, at dosages and for periods of time effective to treat a disease or disorder in the patient. An effective amount of the recited dosages of an ENPP1 agent necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. A selected dosage is determined based on the biological activity of the therapeutic compound which in turn depends on the half-life and the area under the plasma time of the therapeutic compound curve.

### 13. Prophylactic Administration

Armed with the disclosure herein, one skilled in the art would thus appreciate that the prevention of a disease or disorder in a subject encompasses administering to a subject an ENPP1 polypeptide as a preventative measure against the disease or disorder.

The relative amounts of the active ingredient (e.g., soluble ENPP1 polypeptides and fusion proteins thereof), the pharmaceutically acceptable carrier, and any additional ingredients in a formulation disclosed herein will vary, depending upon the identity, size, and condition of the subject treated and further depending upon the route by which the composition is to be administered. By way of example, the composition may comprise between about 0.1% and about 100% (w/w) active ingredient.

### 14. Diseases Relating to Low PPi

In some embodiments, the disclosure contemplates methods of reducing or preventing progression of diseases caused by lower levels of plasma PPi in a subject in need thereof, the method comprising administering to the subject a therapeutically effective amount of the polypeptides disclosed herein to increase the plasma PPi of the subjects to normal (2-3 μM) or above (30-50% higher than) normal levels and then to maintain the plasma PPi at a constant normal or above normal level thereafter. The method further comprises administering additional therapeutic effective amounts at intervals of two days, three days, one week or one month in order to maintain the Plasma PPi of the subject at a constant normal or above normal level to reduce or prevent the progression of pathological calcification or ossification. In certain embodiments, a soluble ENPP1 polypeptide or ENPP1 fusion polypeptide disclosed herein can be used to raise pyrophosphate (PPi) levels in a subject having PPi level lower than normal level (which is around 2 μM). In other embodiments, a soluble ENPP1 polypeptide or ENPP1 fusion polypeptide disclosed herein can be used to reduce or prevent progression of pathological calcification or ossification in a subject having PPi levels lower than normal level. In some embodiments, a soluble ENPP1 polypeptide or ENPP1 fusion polypeptide disclosed herein can be used to treat ENPP1 deficiency (e.g., GACI and ARHR2) manifested by a reduction of extracellular PPi concentration in a subject. In certain embodiments, the steady state level of plasma PPi achieved after administration of a first dosage of a soluble ENPP1 polypeptide or ENPP1 fusion polypeptide disclosed herein is maintained for a time period of at least 2 days, at least 4 days, at least a week or at least a month.

In some embodiments, the disclosure contemplates methods of reducing or preventing progression of a disease caused by lower than normal levels of plasma PPi in a subject in need thereof, the method comprising administering to the subject a therapeutically effective amount of a soluble ENPP1 polypeptide or ENPP1 fusion polypeptide disclosed herein (e.g., SEQ ID NOs: 3-5) to increase and/or sustain the plasma PPi of the subjects to a level that is about 90%, 95%, 100%, 105%, 110%, 120%, 130%, 140%, or 150% of the normal PPi level. In certain embodiments, the method further comprises further administration of the polypeptide disclosed herein every two days, three days, one week, or one month in order to maintain the plasma PPi levels at a level that is about 90%, 95%, 100%, 105%, 110%, 120%, 130%, 140%, or 150% of the normal PPi level, thus preventing the progression of pathological calcification or ossification.

### 15. Treatment/Indications

The recited dosages of an ENPP1 agent disclosed herein may be used in methods of treating, reversing, or preventing progression of diseases associated with an ENPP1 deficiency or an ABCC6 deficiency as disclosed herein.

In an aspect, the disclosure relates to administering to a subject having an ENPP1 deficiency or an ABCC6 deficiency, an ENPP1 agent at a dose of about 0.2 mg per kilogram of the subject, about 0.6 mg per kilogram of the subject, or about 1.8 mg per kilogram of the subject in order to restore a physiological level of ENPP1 in the plasma or tissues of the subject.

In an aspect, the disclosure relates to administering to a subject having an ENPP1 deficiency or an ABCC6 deficiency, an ENPP1 agent at a dose of about 0.2 mg per kilogram of the subject, about 0.6 mg per kilogram of the subject, or about 1.8 mg per kilogram of the subject in order to restore a physiological level of Pi and/or PPi in the plasma of the subject. The physiological level of Pi and PPi in human serum (and in mammals generally) is 1-3 mM and 2-3 μM respectively.

In an aspect, the disclosure relates to a method for preventing progression of or reducing vascular calcification in a subject with ENPP1 Deficiency or with ABCC6 deficiency, the method comprising: to thereby prevent the progression of or reduce vascular calcification in the subject, the method comprising: administering to the subject an ENPP1 agent at a dose of about 0.2 mg per kilogram of the subject, about 0.6 mg per kilogram of the subject, or about 1.8 mg per kilogram of the subject, to thereby prevent the progression of or reduce vascular calcification in the subject.

In an aspect, the disclosure relates to a method for preventing the progression of or reducing pathological calcification in a subject with ENPP1 Deficiency or with ABCC6 deficiency, the method comprising: administering to the subject an ENPP1 agent at a dose of about 0.2 mg per kilogram of the subject, about 0.6 mg per kilogram of the subject, or about 1.8 mg per kilogram of the subject, to thereby prevent the progression of or reduce pathological calcification in the subject.

The recited dosages of an ENPP1 agent disclosed herein may be used in methods of treating, reversing, or preventing progression of diseases associated with pathological calcification as disclosed herein.

In an aspect, the disclosure relates to administering to a subject having pathological calcification an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject in order to restore a physiological level of ENPP1 in the plasma or tissues of the subject.

In an aspect, the disclosure relates to administering to a subject having pathological calcification an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject in order to restore a physiological level of Pi and/or PPi in the plasma of the subject. The physiological level of Pi and PPi in human serum (and in mammals generally) is 1-3 mM and 2-3 μM respectively.

In an aspect, the disclosure relates to a method for preventing progression of or reducing vascular calcification in a subject, the method comprising: to thereby prevent the progression of or reduce vascular calcification in the subject, the method comprising: administering to the subject an ENPP1 agent at a dose of about 0.2 mg per kilogram of the subject, or about 0.4 mg per kilogram of the subject, or about 0.6 mg per kilogram of the subject, or about 0.4 mg per kilogram of the subject, or about 1.8 mg per kilogram of the subject, to thereby prevent the progression of or reduce vascular calcification in the subject.

In an aspect, the disclosure relates to a method for preventing the progression of or reducing pathological calcification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce pathological calcification in the subject.

In an aspect, the disclosure relates to a method for preventing the progression of or reducing tissue calcification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject.

In an aspect, the disclosure relates to a method for preventing the progression of or reducing pathological ossification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject.

In an aspect, the disclosure relates to a method for preventing the progression of or reducing tissue calcification in a subject with ENPP1 Deficiency or with ABCC6 deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject.

In an aspect, the disclosure relates to a method for preventing the progression of or reducing pathological ossification in a subject with ENPP1 Deficiency or with ABCC6 deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject.

In an aspect, the disclosure relates to a method for increasing circulating pyrophosphate (PPi) in a subject with ENPP1 Deficiency or with ABCC6 deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase circulating PPi in the subject.

In an aspect, the disclosure relates to a method for increasing pyrophosphatase activity in a subject with ENPP1 Deficiency or with ABCC6 deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase circulating PPi in the subject.

In an aspect, the disclosure relates to a method for ameliorating one or more symptoms of ENPP1 Deficiency or ABCC6 deficiency in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby ameliorate one or more symptoms of ENPP1 Deficiency or one or more symptoms of ABCC6 deficiency in the subject.

In an aspect, the disclosure relates to a method for treating a subject with ENPP1 Deficiency or with ABCC6 deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat the subject.

In certain aspects, the present disclosure relates to the use of an ENPP1 agent, such as a polypeptide having amino acid sequences as set forth in, e.g., SEQ ID NOs: 2, 3, 4, and 5.

In certain embodiments, the pathological calcification is selected from the group consisting of kidney and bladder stones, dental pulp stones, gall stones, salivary gland stones, chronic calculous prostatitis, testicular microliths, calcification in hemodialysis patients, atherosclerosis, malacoplakia, scleroderma (systemic sclerosis), calcinosis cutis, calcific aortic stenosis, calcific tendonitis, synovitis and arthritis, diffuse interstitial skeletal hyperostosis, juvenile dermatomyositis, Generalized Arterial Calcification of Infancy (GACI), Ossification of the Posterior Longitudinal Ligament (OPLL), hypophosphatemic rickets, autosomal hypophosphatemic rickets (ARHR2), osteoarthritis, calcification of atherosclerotic plaques, Chronic Kidney Disease (CKD), End Stage Renal Disease (ESRD), Pseudoxanthoma elasticum (PXE), ankylosing spondylitis, hardening of the arteries, calciphylaxis, and systemic lupus erythematosus.

In certain embodiments, the pathological calcification is selected from the group consisting of Pseudoxanthoma elasticum (PXE) and calcification of atherosclerotic plaques.

In certain embodiments, the pathological calcification is selected from the group consisting of idiopathic infantile arterial calcification (IIAC) and calcification of atherosclerotic plaques.

In some embodiments, the disclosure contemplates methods of reducing or preventing progression of ectopic calcification of soft tissue, including reducing, ameliorating, or preventing vascular calcification, in a subject in need thereof, the method comprising administering to the subject a therapeutically effective amount of a soluble ENPP1 polypeptide or ENPP1 fusion polypeptide disclosed herein.

In some embodiments, the disclosure contemplates methods of reducing or preventing progression of diseases caused by an ENPP1 deficiency (e.g., GACI and ARHR2), the method comprising administering to the subject a therapeutically effective amount of a soluble ENPP1 polypeptide or ENPP1 fusion protein disclosed herein (e.g., SEQ ID NOs: 2, 3, 4, and 5). In some embodiments, the ENPP1 deficiency is GACI. In some embodiments, the ENPP1 deficiency is ARHR2.

In some embodiments, the disclosure contemplates methods of reducing or preventing progression of diseases caused by an ABCC6 deficiency (e.g., PXE), the method comprising administering to the subject a therapeutically effective amount of a soluble ENPP1 polypeptide or ENPP1 fusion protein disclosed herein (e.g., SEQ ID NOs: 2, 3, 4, and 5). In some embodiments, the ABCC6 deficiency is PXE. In some embodiments, subjects having ABCC6 deficiency exhibit symptoms similar to a person diagnosed with GACI or ARHR2.

In some embodiments, the disclosure contemplates methods of reducing or preventing progression of diseases caused by pathological calcification (e.g., GACI and ARHR2), the method comprising administering to the subject a therapeutically effective amount of a soluble ENPP1 polypeptide or ENPP1 fusion protein disclosed herein (e.g., SEQ ID NOs: 2, 9, 10, and 11). In some embodiments, the subject exhibiting pathological calcification has ENPP1 deficiency. In some embodiments subject exhibiting pathological calcification has ABCC6 deficiency.

In certain embodiments, the polypeptide is a secreted product of an ENPP1 precursor protein expressed in a mammalian cell. In other embodiments, the ENPP1 precursor protein comprises a signal peptide sequence and an ENPP1 polypeptide, wherein the ENPP1 precursor protein undergoes proteolytic processing to the polypeptide disclosed herein. In some embodiments, in the ENPP1 precursor protein the signal peptide sequence is conjugated to the ENPP1 polypeptide N-terminus. Upon proteolysis, the signal sequence is cleaved from the ENPP1 precursor protein to provide the ENPP1 polypeptide. In certain embodiments, the signal peptide sequence is selected from the group consisting of ENPP1 signal peptide sequence, ENPP2 signal peptide sequence, ENPP7 signal peptide sequence, and ENPP5 signal peptide sequence.

It will be appreciated by one of skill in the art, when armed with the present disclosure including the methods detailed herein, that the disclosure is not limited to treatment of a disease or disorder once it is established. Particularly, the symptoms of the disease or disorder need not have manifested to the point of detriment to the subject; indeed, the disease or disorder need not be detected in a subject before treatment is administered. That is, significant pathology from disease or disorder does not have to occur before the present ENPP1 polypeptides may provide benefit.

In certain aspects, the disclosure relates to methods for preventing diseases and disorders in a subject, in that a soluble ENPP1 polypeptide or ENPP1 fusion polypeptide disclosed herein can be administered to a subject prior to the onset of the disease or disorder, thereby preventing the disease or disorder from developing. Therefore, the disclosure relates to methods for preventing or delaying onset, or reducing progression or growth, of a disease or disorder in a subject, comprising administering an ENPP1 polypeptide to a subject prior to detection of the disease or disorder. In certain embodiments, the ENPP1 polypeptide is administered to a subject with a strong family history of the disease or disorder, thereby preventing or delaying onset or progression of the disease or disorder.

### 16. ENPP1 Polypeptide Sequences

TABLE 1
Sequences

| SEQ | | |
|---|---|---|
| ID | | |
| NO | Sequence | Description |
| 1 | 1 MERDGCAGGG SRGGEGGRAP REGPAGNGRD RGRSHAAEAP GDPQAAASLL | Full, |
| | 51 APMDVGEEPL EKAARARTAK DPNTYKVLSL VLSVCVLTTI LGCIFGLKPS | unprocessed |
| | 101 CAKEVKSCKG RCFERTFGNC RCDAACVELG NCCLDYQETC IEPEHIWTCN | amino acid |
| | 151 KFRCGEKRLT RSLCACSDDC KDKGDCCINY SSVCQGEKSW VEEPCESINE | sequence |
| | 201 PQCPAGFETP PTLLFSLDGF RAEYLHTWGG LLPVISKLKK CGTYTKNMRP | of wild- |
| | 251 VYPTKTFPNH YSIVTGLYPE SHGIIDNKMY DPKMNASFSL KSKEKFNPEW | type ENPP1 |
| | 301 YKGEPIWVTA KYQGLKSGTF FWPGSDVEIN GIFPDIYKMY NGSVPFEERI | precursor |
| | 351 LAVLQWLQLP KDERPHFYTL YLEEPDSSGH SYGPVSSEVI KALQRVDGMV | protein |
| | 401 GMLMDGLKEL NLHRCLNLIL ISDHGMEQGS CKKYIYLNKY LGDVKNIKVI | |
| | 451 YGPAARLRPS DVPDKYYSFN YEGIARNLSC REPNQHFKPY LKHFLPKRLH | |
| | 501 FAKSDRIEPL TFYLDPQWQL ALNPSERKYC GSGFHGSDNV FSNMQALFVG | |
| | 551 YGPGFKHGIE ADTFENIEVY NLMCDLLNLT PAPNNGTHGS LNHLLKNPVY | |
| | 601 TPKHPKEVHP LVQCPFTRNP RDNLGCSCNP SILPIEDFQT QFNLTVAEEK | |
| | 651 IIKHETLPYG RPRVLQKENT ICLLSQHQFM SGYSQDILMP LWTSYTVDRN | |
| | 701 DSFSTEDFSN CLYQDFRIPL SPVHKCSFYK NNTKVSYGFL SPPQLNKNSS | |
| | 751 GIYSEALLTT NIVPMYQSFQ VIWRYFHDTL LRKYAEERNG VNVVSGPVFD | |
| | 801 FDYDGRCDSL ENLRQKRRVI RNQEILIPTH FFIVLTSCKD TSQTPLHCEN | |
| | 851 LDTLAFILPH RTDNSESCVH GKHDSSWVEE LLMLHRARIT DVEHITGLSF | |
| | 901 YQQRKEPVSD ILKLKTHLPT FSQED | |
| 2 | 1 PSCAKEVKSC KGRCFERTFG NCRCDAACVE LGNCCLDYQE TCIEPEHIWT | The processed |
| | 51 CNKFRCGEKR LTRSLCACSD DCKDKGDCCI NYSSVCQGEK SWVEEPCESI | (mature) |
| | 101 NEPQCPAGFE TPPTLLFSLD GFRAEYLHTW GGLLPVISKL KKCGTYTKNM | extracellular |
| | 151 RPVYPTKTFP NHYSIVTGLY PESHGIIDNK MYDPKMNASF SLKSKEKFNP | ENPP1 |
| | 201 EWYKGEPIWV TAKYQGLKSG TFFWPGSDVE INGIFPDIYK MYNGSVPFEE | polypeptide |
| | 251 RILAVLQWLQ LPKDERPHFY TLYLEEPDSS GHSYGPVSSE VIKALQRVDG | sequence |
| | 301 MVGMLMDGLK ELNLHRCLNL ILISDHGMEQ GSCKKYIYLN KYLGDVKNIK | |
| | 351 VIYGPAARLR PSDVPDKYYS FNYEGIARNL SCREPNQHFK PYLKHFLPKR | |
| | 401 LHFAKSDRIE PLTFYLDPQW QLALNPSERK YCGSGFHGSD NVFSNMQALF | |
| | 451 VGYGPGFKHG IEADTFENIE VYNLMCDLLN LTPAPNNGTH GSLNHLLKNP | |
| | 501 VYTPKHPKEV HPLVQCPFTR NPRDNLGCSC NPSILPIEDF QTQFNLTVAE | |
| | 551 EKIIKHETLP YGRPRVLQKE NTICLLSQHQ FMSGYSQDIL MPLWTSYTVD | |
| | 601 RNDSFSTEDF SNCLYQDFRI PLSPVHKCSF YKNNTKVSYG FLSPPQLNKN | |
| | 651 SSGIYSEALL TTNIVPMYQS FQVIWRYFHD TLLRKYAEER NGVNVVSGPV | |
| | 701 FDFDYDGRCD SLENLRQKRR VIRNQEILIP THFFIVLTSC KDTSQTPLHC | |
| | 751 ENLDTLAFIL PHRTDNSESC VHGKHDSSWV EELLMLHRAR ITDVEHITGL | |
| | 801 SFYQQRKEPV SDILKLKTHL PTFSQED | |
| 3 | FTAGLKPSCAKEVKSCKGRCFERTFGNCRCDAACVELGNCCLDYQETCI | ENPP1-linker- |
| | EPEHIWTCNKFRCGEKRLTRSLCACSDDCKDKGDCCINYSSVCQGEKSWVEEPCE | hIgG1 Fc |
| | SINEPQCPAGFETPPTLLFSLDGFRAEYLHTWGGLLPVISKLKKCGTYTKNMRPVY | construct |
| | PTKTFPNHYSIVTGLYPESHGIIDNKMYDPKMNASFSLKSKEKFNPEWYKGEPIW | |
| | VTAKYQGLKSGTFFWPGSDVEINGIFPDIYKMYNGSVPFEERILAVLQWLQLPKD | |
| | ERPHFYTLYLEEPDSSGHSYGPVSSEVIKALQRVDGMVGMLMDGLKELNLHRCL | |
| | NLILISDHGMEQGSCKKYIYLNKYLGDVKNIKVIYGPAARLRPSDVPDKYYSFNY | |
| | EGIARNLSCREPNQHFKPYLKHFLPKRLHFAKSDRIEPLTFYLDPQWQLALNPSER | |
| | KYCGSGFHGSDNVFSNMQALFVGYGPGFKHGIEADTFENIEVYNLMCDLLNLTP | |
| | APNNGTHGSLNHLLKNPVYTPKHPKEVHPLVQCPFTRNPRDNLGCSCNPSILPIED | |
| | FQTQFNLTVAEEKIIKHETLPYGRPRVLQKENTICLLSQHQFMSGYSQDILMPLWT | |
| | SYTVDRNDSFSTEDFSNCLYQDFRIPLSPVHKCSFYKNNTKVSYGFLSPPQLNKNS | |
| | SGIYSEALLTTNIVPMYQSFQVIWRYFHDTLLRKYAEERNGVNVVSGPVFDFDYD | |
| | GRCDSLENLRQKRRVIRNQEILIPTHFFIVLTSCKDTSQTPLHCENLDTLAFILPHRT | |
| | DNSESCVHGKHDSSWVEELLMLHRARITDVEHITGLSFYQQRKEPVSDILKLKTH | |
| | LPTFSQEDLINDKTHTCPPCPAPELLGGPSVFLFPPKPKDTLMISRTPEVTCVV | |
| | VDVSHEDPEVKFNWYVDGVEVHNAKTKPREEQYNSTYRVVSVLTVLHQDW | |
| | LNGKEYKCKVSNKALPAPIEKTISKAKGQPREPQVYTLPPSREEMTKNQVSL | |
| | TCLVKGFYPSDIAVEWESNGQPENNYKTTPPVLDSDGSFFLYSKLTVDKSRW | |
| | QQGNVFSCSVMHEALHNHYTQKSLSLSPGK | |
| 4 | GLKPSCAKEVKSCKGRCFERTFGNCRCDAACVELGNCCLDYQETCIEPE | ENPP1-linker- |
| | HIWTCNKFRCGEKRLTRSLCACSDDCKDKGDCCINYSSVCQGEKSWVEEPCESIN | hIgG1 Fc |
| | EPQCPAGFETPPTLLFSLDGFRAEYLHTWGGLLPVISKLKKCGTYTKNMRPVYPT | construct |
| | KTFPNHYSIVTGLYPESHGIIDNKMYDPKMNASFSLKSKEKFNPEWYKGEPIWVT | |
| | AKYQGLKSGTFFWPGSDVEINGIFPDIYKMYNGSVPFEERILAVLQWLQLPKDERP | |
| | HFYTLYLEEPDSSGHSYGPVSSEVIKALQRVDGMVGMLMDGLKELNLHRCLNLIL | |
| | ISDHGMEQGSCKKYIYLNKYLGDVKNIKVIYGPAARLRPSDVPDKYYSFNYEGIA | |
| | RNLSCREPNQHFKPYLKHFLPKRLHFAKSDRIEPLTFYLDPQWQLALNPSERKYC | |
| | GSGFHGSDNVFSNMQALFVGYGPGFKHGIEADTFENIEVYNLMCDLLNLTPAPNN | |
| | GTHGSLNHLLKNPVYTPKHPKEVHPLVQCPFTRNPRDNLGCSCNPSILPIEDFQTQ | |
| | FNLTVAEEKIIKHETLPYGRPRVLQKENTICLLSQHQFMSGYSQDILMPLWTSYTV | |
| | DRNDSFSTEDFSNCLYQDFRIPLSPVHKCSFYKNNTKVSYGFLSPPQLNKNSSGIYS | |
| | EALLTTNIVPMYQSFQVIWRYFHDTLLRKYAEERNGVNVVSGPVFDFDYDGRCD | |
| | SLENLRQKRRVIRNQEILIPTHFFIVLTSCKDTSQTPLHCENLDTLAFILPHRTDNSE | |
| | SCVHGKHDSSWVEELLMLHRARITDVEHITGLSFYQQRKEPVSDILKLKTHLPTFS | |
| | QEDLINDKTHTCPPCPAPELLGGPSVFLFPPKPKDTLMISRTPEVTCVVVDVS | |
| | HEDPEVKFNWYVDGVEVHNAKTKPREEQYNSTYRVVSVLTVLHQDWLNGK | |
| | EYKCKVSNKALPAPIEKTISKAKGQPREPQVYTLPPSREEMTKNQVSLTCLV | |
| | KGFYPSDIAVEWESNGQPENNYKTTPPVLDSDGSFFLYSKLTVDKSRWQQGN | |
| | VFSCSVMHEALHNHYTQKSLSLSPGK | |
| 5 | PSCAKEVKSCKGRCFERTFGNCRCDAACVELGNCCLDYQETCIEPEHIW | ENPP1-linker- |
| | TCNKFRCGEKRLTRSLCACSDDCKDKGDCCINYSSVCQGEKSWVEEPCESINEPQ | hIgG1 Fc |
| | CPAGFETPPTLLFSLDGFRAEYLHTWGGLLPVISKLKKCGTYTKNMRPVYPTKTFP | construct |
| | NHYSIVTGLYPESHGIIDNKMYDPKMNASFSLKSKEKFNPEWYKGEPIWVTAKYQ | |
| | GLKSGTFFWPGSDVEINGIFPDIYKMYNGSVPFEERILAVLQWLQLPKDERPHFYT | |
| | LYLEEPDSSGHSYGPVSSEVIKALQRVDGMVGMLMDGLKELNLHRCLNLILISDH | |
| | GMEQGSCKKYIYLNKYLGDVKNIKVIYGPAARLRPSDVPDKYYSFNYEGIARNLS | |
| | CREPNQHFKPYLKHFLPKRLHFAKSDRIEPLTFYLDPQWQLALNPSERKYCGSGF | |
| | HGSDNVFSNMQALFVGYGPGFKHGIEADTFENIEVYNLMCDLLNLTPAPNNGTH | |
| | GSLNHLLKNPVYTPKHPKEVHPLVQCPFTRNPRDNLGCSCNPSILPIEDFQTQFNL | |
| | TVAEEKIIKHETLPYGRPRVLQKENTICLLSQHQFMSGYSQDILMPLWTSYTVDRN | |
| | DSFSTEDFSNCLYQDFRIPLSPVHKCSFYKNNTKVSYGFLSPPQLNKNSSGIYSEAL | |
| | LTTNIVPMYQSFQVIWRYFHDTLLRKYAEERNGVNVVSGPVFDFDYDGRCDSLE | |
| | NLRQKRRVIRNQEILIPTHFFIVLTSCKDTSQTPLHCENLDTLAFILPHRTDNSESCV | |
| | HGKHDSSWVEELLMLHRARITDVEHITGLSFYQQRKEPVSDILKLKTHLPTFSQED | |
| | LINDKTHTCPPCPAPELLGGPSVFLFPPKPKDTLMISRTPEVTCVVVDVSHEDP | |
| | EVKFNWYVDGVEVHNAKTKPREEQYNSTYRVVSVLTVLHQDWLNGKEYKC | |
| | KVSNKALPAPIEKTISKAKGQPREPQVYTLPPSREEMTKNQVSLTCLVKGFYP | |
| | SDIAVEWESNGQPENNYKTTPPVLDSDGSFFLYSKLTVDKSRWQQGNVFSCS | |
| | VMHEALHNHYTQKSLSLSPGK | |
| 6 | DKTHTCPPCPAPELLGGPSVFLFPPKPKDTLMISRTPEVTCVVVDVSHED | Human IgG1 Fc |
| | PEVKFNWYVDGVEVHNAKTKPREEQYNSTYRVVSVLTVLHQDWLNGKEYKCK | |
| | VSNKALPAPIEKTISKAKGQPREPQVYTLPPSREEMTKNQVSLTCLVKGFYPSDIA | |
| | VEWESNGQPENNYKTTPPVLDSDGSFFLYSKLTVDKSRWQQGNVFSCSVMHEAL | |
| | HNHYTQKSLSLSPGK | |

```
(Mouse NPP1-NCBI accession NP_001295256.1)
SEQ ID NO: 7
    1 MERDGDQAGH GPRHGSAGNG RELESPAAAS LLAPMDLGEE PLEKAERARP AKDPNTYKVL
  61 SLVLSVCVLT TILGCIFGLK PSCAKEVKSC KGRCFERTFS NCRCDAACVS LGNCCLDFQE
121 TCVEPTHIWT CNKFRCGEKR LSRFVCSCAD DCKTHNDCCI NYSSVCQDKK SWVEETCESI
181 DTPECPAEFE SPPTLLFSLD GFRAEYLHTW GGLLPVISKL KNCGTYTKNM RPMYPTKTFP
241 NHYSIVTGLY PESHGIIDNK MYDPKMNASF SLKSKEKFNP LWYKGQPIWV TANHQEVKSG
301 TYFWPGSDVE IDGILPDIYK VYNGSVPFEE RILAVLEWLQ LPSHERPHFY TLYLEEPDSS
361 GHSHGPVSSE VIKALQKVDR LVGMLMDGLK DLGLDKCLNL ILISDHGMEQ GSCKKYVYLN
421 KYLGDVNNVK VVYGPAARLR PTDVPETYYS FNYEALAKNL SCREPNQHFR PYLKPFLPKR
481 LHFAKSDRIE PLTFYLDPQW QLALNPSERK YCGSGFHGSD NLFSNMQALF IGYGPAFKHG
541 AEVDSFENIE VYNLMCDLLG LIPAPNNGSH GSLNHLLKKP IYNPSHPKEE GFLSQCPIKS
601 TSNDLGCTCD PWIVPIKDFE KQLNLTTEDV DDIYHMTVPY GRPRILLKQH RVCLLQQQQF
661 LTGYSLDLLM PLWASYTFLS NDQFSRDDFS NCLYQDLRIP LSPVHKCSYY KSNSKLSYGF
721 LTPPRLNRVS NHIYSEALLT SNIVPMYQSF QVIWHYLHDT LLQRYAHERN GINVVSGPVF
781 DFDYDGRYDS LEILKQNSRV IRSQEILIPT HFFIVLTSCK QLSETPLECS ALESSAYILP
841 HRPDNIESCT HGKRESSWVE ELLTLHRARV TDVELITGLS FYQDRQESVS ELLRLKTHLP
901 IFSQED
(Cow NPP1-NCBI accession NP_001193141.1)
SEQ ID NO: 8
    1 MERDSCAGGG SRGGEGGRGP REGLAGNGRD PGPGRAAEAS GEPQAAASLL APMDLGEEPL
  61 ERAARARPAK DPNTYKVLSL VLSVCVLTTI LGCIFGLKPS CAKEIKSCKG RCFERTFGNC
121 RCDAACVDLG NCCLDYQETC IEPERIWTCT KFRCGEKRLS RSLCSCSDDC KDKGDCCINH
181 GSVCRGEKSW AEEECDSIDE PQCPAGFETP PTLLFSLDGF RAEYLHTWGG LLPVISKLKT
241 CGTYTKNMRP VYPTKTFPNH YSIVTGLYPE SHGIIDNNIY DPQMNANFAL KNKEKFNPEW
301 YKGEPIWLTA KYQGLKTGTF FWPGSDVKIN GIFPDIYKIY NVSVPFEERI LAILKWLQLP
361 KDERPHFYTL YLEEPDSSGH SYGPVSSEVI RALQRVDNMV GMLMDGLKEL NLHRCLNLIL
421 ISDHGMEQGS CKKYVYLNKY LGDTKDYKVV YGPAARLRPS DVPDKYYSFD YEGIAKNLSC
481 QEPNQHFKPY LKHFLPKRLH FAKNDRIERL TFYLDPQWQL ALNPSERKYC GGGFHGSDNT
541 FLNMQALFIG YGPGFKHSTE VDSFENIEVY NLMCDLLNLT PAPNNGTHGS LNHLLSNPVY
601 TPKHPKEVRP LVQCPFTRAP RESLDCSCDP SILPIVDFQT QLNLTMAEEK TIKRGALPYG
661 RPRVLQNSTV CLLYQHQFVS GYSRDILMPL WTSYTIGRND SFSTEDFSNC LYQDLRIPLS
721 PVHKCSFYKN NAKLSYGLLS PPQLHKGSSQ VYSEALLTTN IVPMYQSFQV IWHYLHGTLL
781 QRYAEERNGL NVVSGPVFDS DYDGRYDSLE TLKQNSKIIR NLEVLIPTHF FLVLTSCKNT
841 SQTPLQCENL DAMAFILPHK TDNSESCAHG KHESLWVEEL LKLHTARITD VEHITGLSFY
901 QERKEPISDI LKLKTHLPTF NQED
(Rabbit NPP1-NCBI accession NP_001162404.1)
SEQ ID NO: 9
    1 MERDGCAGGG SRGGEGGRAP REGPAGNSRD PGRSHAAEAP GNPQAAASLL APMDVGEEPL
  61 EKAARARTAK DPNTYKVLSL VLSVCVLTTI LGCIFGLKPS CAKEVKSCKG RCFERTFGNC
121 RCDAACVELG NCCLDYQETC IEPEHIWTCN KFRCGEKRLT RSLCACSDDC KDQGDCCINY
181 SSVCQGEKSW VEEPCESINE PQCPAGFETP PTLLFSLDGF RAEYLHTWGG LLPVISKLKK
241 CGTYTKNMRP VYPTKTFPNH YSIVTGLYPE SHGIIDNKMY DPKMNASFSL KSKEKFNPEW
301 YKGEPIWVTA KYQGLKSGTF FWPGSDVEIN GIFPDIYKMY NGSVPFEERI LAVLQWLQLP
361 KDERPHFYTL YLEEPDSSGH SYGPVSSEVI KALQRVDNMV GMLMDGLKEL NLHRCLNLIL
421 VSDHGMEQGS CKKYIYLNKY LGDVKNIKVI YGPAARLRPS DVPDKYYSFN YEGIARNLSC
481 REPNQHFKPY LKHFLPKRLH FAKSDRIEPL TFYLDPQWQL ALNPSERKYC GSGFHGSDNI
541 FSNMQALFVG YGPGFKHGIE VDTFENIEVY NLMCDLLNLT PAPNNGTHGS LNHLLKNPVY
601 TPKHPKEVHP LIQCPFTRNP RDNLGCSCNP SILPIEDFQT QFNLTVAEEK NIKHETLPYG
661 RPRVLQKKNT ICLLSQHQFM SGYSQDILMP LWTSYTVDRN DSFSTEDFSN CLYQDFRISL
721 SPVHKCSFYK NNTKVSYGFL SPPQLNKNSR GIYSEALLTT NIVPMYQSFQ VIWRYFHDTL
781 LRKYAEERNG VNVVSGPVFD FDYDGRYDSL EILRQKRRVI RNQEILIPTH FFIVLTSCKD
841 ASQTPLHCEN LDTLAFILPH RTDNSESCLH GKHESSWVEE LLMLHRARIT DVEHITGLSF
901 YQQRKEPVSD ILKLKTHLPT FSQED
(Baboon NPP1-NCBI accession NP_001076211.2)
SEQ ID NO: 10
    1 MERDGCAGGG SQGGGKGGRG PREGLAGNGR DPSHGQASEA PGDPQAAASL LAPMDLGEEP
  61 LEKAAGARPA KDPNTYKVLS LVLSVCVLTT ILGCIFGLKP SCAKEVKSCK GRCFERTFGN
121 CRCDVACVDL GNCCLDYQET CIEPERIWTC NKFRCGEKRL SRSLCACSDD CKERGDCCIN
181 YSAVCQGEKS WVEETCENIN EPQCPEGFEM PPTLLFSLDG FRAEYLHTWG GLLPVISKLK
241 KCGTYAKNMR PVYPTKTFPN HYSIVTGLYP ESHGIIDNKM YDPKMNASFS LKSKEKFNPE
301 WYKGEPIWLT AKYQGLRSGT FFWPGSDVKI NGIFPDIYKI YNGSVPFEER ILAILKWLRL
361 PKDERPHFYT LYLEEPDSSG HSYGPVSSEV IKALQRVDNM VGMLMDGLKE LNLHQCLNLI
421 LISDHGMEQG SCKKYIYLNK YLGDTKNIKV IYGPAARLRP SDVPEKYYSF NYENIARNLS
481 CREPNQHFKP YLKHFLPKRL HFAKSDRIEP LTFYLDPQWQ LALSPSERKY CGSGFHGSDN
541 VFSNMQALFV GYGPGFQHGI EVDSFENIEV YNLMCDLLNL TPAPNNGTHG SLNHLLKNPI
601 YTPKHPKEVQ PSVQCPLAGS PRDSLGCSCN PSILPIVDFQ TQFNLTTAEE KNINRASLPY
661 GRPRLLQKKS SVCLLYQHQF VSGYSHDVLM PLWTSYTVNR NDSFSTEDFS NCLYQDLRIS
721 FSPIHNCSFY KNNAKLSYGF LSPPQLSKDS SQIYSEALLT SNIVPMYQSF QVIWRYFHDT
781 LLQRYAEERN SINVVSGPVF DSDYDGRYDS SEALKRNRRV IRNQEILIPT HFFIVITSCK
841 NTSQTPLQCD NLDPLAFILP HRSDNSESCV HEKRESSWIE ELLMMHRARI MDVEHITGLS
901 FYQERKEPVS DILKLKTHLP TVSQED
```

### 17. Treatment Protocols

The following protocols may be used as guidance for ENPP1 enzyme replacement therapy at the recited dosages of the ENPP1 agent, as determined to be appropriate by the medical practitioner. Treatment protocols set forth herein rely on the use of abbreviated terms, whose full names are set forth in Table 2.

a. Prohibited Medication or Therapy**

A patient subject to treatment with an ENPP1 agent should discontinue use of oral phosphate and vitamin D3 or analogs prior to the first dose of an ENPP1 agent (e.g., at least 14 days prior to the first dose) and use is prohibited throughout the treatment period. Oral phosphate treatment may be tapered to avoid hypercalciuria. Vitamin D metabolites or analogs may be discontinued without titration. Oral bisphosphonates should be withdrawn 6 months prior to the first dose.

### Additional Prohibited Medications Throughout the Treatment Period are:

bisphosphonates
anti-FGF23 (e.g. burosumab)
calcimimetics
antacids
systemic corticosteroids
PTH suppressors
   b. Treatment Frequency

An ENPP1 agent is administered at least once or twice bimonthly, at least once or twice monthly, three times monthly, at least once or twice weekly.

In some embodiments, the ENPP1 agent is administered once a week. In some embodiments, the ENPP1 agent is administered twice a week. In some embodiments, the ENPP1 agent is administered once every two weeks. In some embodiments, the ENPP1 agent is administered once every three weeks. In some embodiments, the ENPP1 agent is administered once a month. In some embodiments, the ENPP1 agent is administered once every five weeks. In some embodiments, the ENPP1 agent is administered once every six weeks. In some embodiments, the ENPP1 agent is administered once every seven weeks. In some embodiments, the ENPP1 agent is administered once every two months. In some embodiments, the ENPP1 agent is administered once every nine weeks. In some embodiments, the ENPP1 agent is administered once every ten weeks. In some embodiments, the ENPP1 agent is administered once every eleven weeks. In some embodiments, the ENPP1 agent is administered once every three months.

The first dose of an ENPP1 agent may be administered on Day 1. On Days 8 to 29 and thereafter, the ENPP1 agent is administered to a subject at a selected dose of ENPP1 agent mg/kg doses twice weekly. The dose may be administered at approximately the same time on each dosing day. The site of injection is alternated, with no site within 2 inches of any prior site of injection within the prior 2 weeks.

A selected dose of an ENPP1 agent of 0.2 mg/kg, 0.6 mg/kg, or 1.8 mg/kg is administered SC over a time period determined by a medical practitioner skilled in the field of ENPP1 replacement therapy. The first dose of an ENPP1 agent may be administered on Day 1. After the first dose, a subject may be observed for 7 days to monitor safety and to collect PK samples. On Days 8 to 29, a subject receives a selected dose twice weekly. Administration of an ENPP1 agent at a selected dose is continued as considered appropriate by the medical professional.

A subject may receive 8 doses of an ENPP1 agent over the course of a 29 day period of time, resulting in an exposure of 1.6 mg, 4.8 mg, and 14.4 mg per 29 days, respectively, for dose amounts of 0.2 mg/kg, 0.6 mg/kg, and 1.8 mg/kg.

Like the endogenous ENPP1 enzyme, an ENPP1 agent cleaves ATP to generate AMP and PPi, thereby increasing plasma PPi levels and into AMP which CD73 coverts rapidly to adenosine. Replacement of the endogenous human enzyme is intended to correct the inherent deficiency and allow for improved health and mitigation of clinical complications associated with ENPP1 Deficiency.

c. Evaluation of Treatment
i. Pharmacokinetics
An ENPP1 agent plasma concentration-time profiles and determination of noncompartmental PK parameters (including Tmax, Cmax, AUClast, AUCtau, AUCinf, T1/2, Cmin, Cl/F and V/F)
Assess linearity between ascending ENPP1 agent SC doses and PK parameters.
   ii. Pharmacodynamics
Change from baseline for plasma PPi levels, serum phosphate, plasma intact FGF23 levels, TmP/GFR (adjusted to creatinine clearance)
Assess linearity between ascending ENPP1 agent SC doses and PD parameters
Correlate the changes in PPi with changes in FGF23
Correlate the changes in PPi with changes in TmP/GFR
Correlate the changes in FGF23 with changes in TmP/GFR
   Exploration of presence and changes of blood and urine biomarkers
   Plasma and urine creatinine (used to calculate the renal clearance of phosphate)
   Serum 1.25(OH)2D, plasma ionized and total calcium, parathyroid hormone
   Bone biomarkers: serum alkaline phosphatase (ALP), bone-specific ALP (BALP), carboxy terminal cross-linked telopeptide of type I collagen (CTx), and procollagen type 1 N-terminal propeptide (P1NP)
      iii. Efficacy

In order to assess treatment efficacy, determination of one or more of the following physical parameters may be made prior to and during treatment.
Baseline Skeletal
Bone density using DEXA
Na¹⁸F-PET/HR-pQCT (or HR-CT)
Baseline arterial and organ calcification
Na¹⁸F-PET/HR-pQCT (or HR-CT)
Echocardiogram
Baseline cardiovascular and peripheral vascular reactivity function
Stress Doppler echocardiography
ECG
Ankle-brachial Index
Pulse Wave Velocity
Peripheral arterial tonometry (PAT)
HRpQCT (or HR-CT) with and without contrast
Baseline neurological function
NIH Stroke Score
Neurological exam
Baseline pulmonary function
Standard pulmonary function test
Baseline performance outcomes
2 MWT, 6 MWT
Handheld dynamometry, grip strength, Range of Motion
Hearing tests: Physical exam and otoscopy, immittance audiometry (tympanometry), Pure Tone Audiometry (PTA), High Frequency Audiometry (HFA)
Baseline patient, clinician, and caregiver outcomes
Patient and Physician Global Impression of Change (CGI-C and CGI-S)
Gross Motor Function Classification System—Expanded and Revised
PROMIS Pain Interference and pain intensity
   PROMIS Fatigue, mobility, cognitive impact, upper extremity
Western Ontario and McMaster University Osteoarthritis Index (WOMAC) Stiffness Score
Baseline renal calcification as measured by renal ultrasound
Baseline bone histomorphometry using bone biopsy (optional)
Baseline optical coherence tomography of aorta, coronary arteries, carotid arteries, and renal arteries and vascular beds.
   iv. Safety and Immunogenicity

Safety assessments may be summarized at Baseline and at each observed time point. Safety variables include:
Incidence, frequency, and severity of adverse events (AEs), treatment-related AEs, and serious adverse events (SAEs)
Vital signs and weight
Physical examinations
Estimated glomerular filtration rate (eGFR)
Laboratory tests including chemistry, hematology, and urinalysis, including additional biochemical parameters of interest
Anti-ENPP1-FC antibody testing and dose-limiting toxicities (DLTs)
Incidence of any anti-drug antibodies (ADA)
Incidence of TEAEs associated with hypersensitivity reactions
Concomitant medications
Electrocardiogram (ECG)

TABLE 2

| | |
|---|---|
| 2MWT | 2-minute walk test |
| 6MWT | 6-minute walk test |
| ADA | anti-drug antibodies |
| AE | adverse event |
| ALP | alkaline phosphatase |
| AMP | adenosine monophosphate |
| ARHR2 | autosomal recessive hypophosphatemic rickets type 2 |
| ATP | adenosine triphosphate |
| AUC0-t | Area under the plasma concentration-time curve from time |
| | zero to the time of last measurable concentration |
| AUCinf | Area under the plasma concentration-time curve from time zero to |
| | infinity |
| AUCtau | Area under the concentration-time curve over the dosing interval |
| BALP | bone-specific alkaline phosphatase |
| CaGI | Caregiver Global Impression |
| CGI-C | Clinician Global Impression of Change |
| CL/F | clearance after extravascular administration of drug |
| Cmax | Maximum plasma concentration |
| CTx | carboxy terminal cross-linked telopeptide of type I collagen |
| DSMB | Data Safety Monitoring Board |
| ECG | Electrocardiogram |
| eCRF | electronic case report form |
| ENPP1 | ectonucleotide pyrophosphatase/phosphodiesterase 1 |
| EudraCT | European Union Drug Regulating Authorities Clinical Trials |
| ERT | enzyme replacement therapy |
| FDA | Food and Drug Administration |
| FGF23 | fibroblast growth factor 23 |
| FIH | first in human |
| FIP | first in patient |
| GACI | Generalized Arterial Calcification of Infancy |
| GCP | Good Clinical Practice |
| GLP | Good Laboratory Practice |
| GMFCS- | Gross Motor Function Classification System - Expanded and |
| E and R | Revised |
| HRqCT | high-resolution quantitative computed tomography |
| IB | Investigator Brochure |
| ICF | Informed Consent Form |
| ICH | International Conference on Harmonisation of |
| | Technical Requirements for Registration of Pharmaceuticals |
| | for Human Use |
| ICH E6 | ICH Harmonised Tripartite Guideline: Guideline for Good |
| | Clinical Practice E6 |
| IEC | institutional ethics committee |
| IND | Investigational New Drug (application) |
| INZ-701 | recombinant human ectonucleotide |
| | pyrophosphatase/phosphodiesterase 1 fused to the Fc fragment of IgG1 |
| IRB | Institutional Review Board |
| MAD | multiple ascending dose |
| MTD | maximum tolerated dose |
| NOAEL | no observed adverse effect level |
| PD | pharmacodynamic(s) |
| PK | pharmacokinetic(s) |
| PPi | inorganic pyrophosphate |
| PROMIS | Patient Reported Outcomes Measurement Information System |
| PTH | parathyroid hormone |
| SAE | serious adverse event |
| SAP | Statistical Analysis Plan |
| SC | Subcutaneous |
| T½ | elimination half-life |
| Tmax | time to maximum plasma concentration |
| TEAE | treatment-emergent adverse events |
| TmP/GFR | tubular maximum reabsorption rate of phosphate adjusted for |
| | glomerular filtration rate |
| V/F | apparent volume of distribution after extravascular administration |
| | of drug |
| WOCBP | women of child-bearing potential |
| WOMAC | Western Ontario and McMaster University Osteoarthritis Index |

### EXEMPLIFICATION

The invention now being generally described, it will be more readily understood by reference to the following examples, which are included merely for purposes of illustration of certain embodiments and embodiments of the present invention and are not intended to limit the invention.

### Example 1. Generation of ENPP11 Fusion Proteins

One example of an ENPP1 fusion protein is ENPP1-Fc. However, the exemplification of ENPP1-Fc can be applied to other ENPP1 fusion proteins as set forth herein.

ENPP1-Fc is a recombinant fusion protein that contains the extracellular domains of human ENPP1 (soluble ENPP1) coupled with an Fc fragment of IgG1 (rhENPP1-Fc). The recombinant extracellular domains of ENPP1-Fc contain its catalytic activity and are identical to the native ENPP1 enzyme. ENPP1-Fc is a recombinant human protein produced in CHO cells via a fed batch cell culture process that is free of animal-derived components. The molecular weight of the ENPP1-Fc dimer is approximately 290 kDa; ENPP1-Fc is highly glycosylated and has a pI of approximately 6.0. Like endogenous ENPP1, the primary substrate for ENPP1-Fc is ATP, which is cleaved to AMP and PPi.

In a specific embodiment, soluble ENPP1 protein was fused to a human Fc domain with a linker via a linker (comprising a leucine, isoleucine, and asparagine). Three ENPP1-Fc constructs are shown in Table 1 as SEQ ID NOs: 3, 4, and 5 as purified from CHO cells.

Purification of ENPP1-Fc could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. Following purification of the protein, the catalytic activity of the ENPP1-Fc protein could be evaluated using pNP-TMP as a chromogenic substrate.

### Example 2: Treatment

ENPP1-FC is administered at one of the following selected doses: 0.2 mg/kg, 0.6 mg/kg, and 1.8 mg/kg. Administration is subcutaneous (SC) at least once or twice bimonthly, at least once or twice monthly, three times monthly, at least once or twice weekly.

The first dose of ENPP1-FC may be administered on Day 1. On Days 8 and thereafter, ENPP1-Fc is administered to a subject at a selected dose of ENPP1 agent mg/kg doses twice weekly. The dose may be administered at approximately the same time on each dosing day. The site of injection is alternated, with no site within 2 inches of any prior site of injection within the prior 2 weeks.

A selected dose of ENPP1-FC is one of 0.2 mg/kg, 0.6 mg/kg, or 1.8 mg/kg SC. The first dose of ENPP1-FC may be administered on Day 1. After the first dose, a subject may be observed for 7 days to monitor safety and to collect PK samples. On Days 8 and thereafter, a subject receives a selected dose twice weekly. In some embodiments, on Days 8 and thereafter, a subject receives a selected dose once weekly. Administration of ENPP1-Fc at a selected dose is continued as considered appropriate by the medical professional.

A subject may receive 8 doses of ENPP1-FC over the course of a 29 day period of time, for example, resulting in an exposure of 1.6 mg, 4.8 mg, and 14.4 mg per 29 days, respectively, for dose amounts of 0.2 mg/kg, 0.6 mg/kg, and 1.8 mg/kg. Or a subject may receive more or less than 8 doses, as considered appropriate by a medical profession.

Like the endogenous ENPP1 enzyme, ENPP1-FC cleaves ATP to generate AMP and PPi, thereby increasing plasma PPi levels and into AMP which CD73 coverts rapidly to adenosine. Replacement of the endogenous human enzyme is intended to correct the inherent deficiency and allow for improved health and mitigation of clinical complications associated with ENPP1 Baseline patient, clinician, and caregiver outcomes.

### Example 3: Treatment of a Patient Having an ENPP1 Deficiency

Enpp1-Fc is administered to a patient identified as having an ENPP1 deficiency by subcutaneous injection on Day 1 and twice weekly starting on Day 8 using a select dose as follows.

| | |
|---|---|
| 1 | 0.2 mg/kg |
| 2 | 0.6 mg/kg |
| 3 | 1.8 mg/kg |

ENPP1-Fc is administered at a selected dose of ENPP1-FC is one of 0.2 mg/kg, 0.6 mg/kg, or 1.8 mg/kg SC at least once or twice weekly for a period of time determined by the medical professional. The Patient's response to enzyme replacement is monitored as appropriate, as determined by the medical professional, e.g., by following a reduction in one or more symptoms of ENPP1 deficiency, and/or using guidance provided herein. In some instances, the study period for evaluating the efficacy of treating ENPP1 deficiency using ENPP1-Fc at different dosages is 32 days (Phase I). In some instances, the study period for evaluating the efficacy of treating ENPP1 deficiency using ENPP1-Fc at different dosages is an extended study period of 338 days (Phase II).

The following examples, in the context of the entire specification, provide guidance to determine treatment protocols and efficacy.

### Example 4: Treatment of a Patient Diagnosed with GACI

Enpp1 deficiency may mask as GACI. GACI is a rare disease occurring in infants and involving extensive arterial calcification (Albright, et al., 2015, Nature Comm. 10006).

ENPP1-Fc is administered at a selected dose of ENPP1-FC is one of 0.2 mg/kg, 0.6 mg/kg, or 1.8 mg/kg SC at least twice weekly for a period of time determined by the medical professional. GACI Patient response to enzyme replacement is monitored as appropriate, as determined by the medical professional, e.g., by following a reduction in one or more symptoms of GACI, and/or using guidance provided herein.

The following examples, in the context of the entire specification, provide guidance to determine treatment protocols and efficacy.

### Example 5: Treatment of Patient Diagnosed with ARHR2

Enpp1 deficiency may mask as ARHR2. ARHR2 is a rare skeletal disorder characterized by low levels of plasma PPi and serum phosphate which can result in rickets, repeated fractures of the long bones, rachitic skeletal deformities and impaired growth and development (Ferreira et al 2014, Moran 1975, Rutsch et al 2008).

ENPP1-Fc is administered at a selected dose of ENPP1-FC is one of 0.2 mg/kg, 0.6 mg/kg, or 1.8 mg/kg SC at least twice weekly for a period of time determined by the medical professional. ARHR2 Patient response to enzyme replacement is monitored as appropriate, as determined by the medical professional, e.g., by following a reduction in one or more symptoms of ARHR2, using guidance provided herein.

The following examples, in the context of the entire specification, provide guidance to determine treatment protocols and efficacy.

### Example 6: Treatment of a Patient Having an ABCC6 Deficiency

Enpp1-Fc is administered to a patient identified as having an ABCC6 deficiency by subcutaneous injection on Day 1 and twice weekly starting on Day 8 using a select dose as follows.

| | | |
|---|---|---|
| 1 | 10.2 | mg/kg |
| 2 | 0.6 | mg/kg |
| 3 | 1.8 | mg/kg |

ENPP1-Fc is administered at a selected dose of ENPP1-FC is one of 0.2 mg/kg, 0.6 mg/kg, or 1.8 mg/kg SC at least twice weekly for a period of time determined by the medical professional. The Patient's response to enzyme replacement is monitored as appropriate, as determined by the medical professional, e.g., by following a reduction in one or more symptoms of ABCC6 deficiency, and/or using guidance provided herein.

The following examples, in the context of the entire specification, provide guidance to determine treatment protocols and efficacy.

### Example 7: Treatment of Patients (Female and Male) Diagnosed with PXE Having an ABCC6 Deficiency

A Phase 1/2, Open-Label, Multiple Ascending Dose Study is performed in order to Evaluate the Safety, Tolerability, Pharmacokinetics, and Pharmacodynamics of INZ-701. This is followed by an Open-Label Long-Term Extension Period in Adults With ABCC6 Deficiency Manifesting as Pseudoxanthoma Elasticum (PXE).

| Arm | Intervention/treatment |
|---|---|
| Experimental: INZ-701 | Drug: INZ-701 |
| The study design | Recombinant fusion |
| is a MAD 3 + 3 with 3 | protein that contains |
| dose cohorts. The planned | the extracellular domains of human |
| doses will be 0.2 mg/kg, | ENPP1 coupled with an Fc fragment |
| 0.6 mg/kg, and 1.8 mg/kg | from an immunoglobulin gamma-1 |
| administered via subcutaneous | (IgG1) antibody (rhENPP1-Fc) |
| injection twice weekly. | |

### Outcome Measures

Measures of Primary and Secondary Outcomes are as follows.

### Primary Outcome Measure:

1. Number of Treatment Emergent Adverse Events (TEAEs) [Time Frame: 32 days (Dose Evaluation Period)]
Treatment-emergent AEs are defined as any AE occurring from the first dose of INZ-701 through 30 days after the last dose of INZ-701.
2. Number of Treatment Emergent Adverse Events (TEAEs) [Time Frame: 52 weeks (Day 1 through Safety Follow-up Visit)]
Treatment-emergent AEs are defined as any AE occurring from the first dose of INZ-701 through 30 days after the last dose of INZ-701.

### Secondary Outcome Measures:

1. Incidence of Anti-Drug Antibodies (ADAs) [Time Frame: 32 days (Dose Evaluation Period)]

The presence of ADAs will be assessed and, if present, further evaluation will determine specificity and subtypes.
2. Incidence of Anti-Drug Antibodies (ADAs) [Time Frame: 52 weeks (Day 1 through Safety Follow-up Visit)]

The presence of ADAs will be assessed and, if present, further evaluation will determine specificity and subtypes.
3. Area under the Plasma Concentration versus Time Curve (AUC) of INZ-701 [Time Frame: 32 days (Dose Evaluation Period)]

For each subject, variation of concentration of INZ-701 in the plasma will be measured via a series of blood samples obtained throughout the study, comparing the subject's baseline value over time.
4. Maximum Plasma Concentration (Cmax) of INZ-701 [Time Frame: 32 days (Dose Evaluation Period)]

For each subject, the maximum concentration of INZ-701 in the plasma will be measured via a series of blood samples obtained throughout the study, comparing the subject's baseline value over time.
5. Systemic Clearance of INZ-701 [Time Frame: 32 days (Dose Evaluation Period)]

For each subject, clearance of INZ-701 from the body will be measured via a series of blood samples obtained throughout the study, comparing the subject's baseline value over time.
6. Change from Baseline in Plasma Inorganic Pyrophosphate (PPi) Levels [Time Frame: 32 days (Dose Evaluation Period)]

For each subject, plasma PPi will be measured via a series of blood samples obtained throughout the study, comparing the subject's baseline value over time.
7. Change from Baseline in Plasma Inorganic Pyrophosphate (PPi) Levels [Time Frame: 52 weeks (Baseline through Safety Follow-up Visit)].

For each subject, plasma PPi will be measured via a series of blood samples obtained throughout the study, comparing the subject's baseline value over time.

### Eligibility Criteria

Ages Eligible for Study: 18 Years to 64 Years
Sexes Eligible for Study: All
Gender Based: No
Accepts Healthy Volunteers: No

### Criteria for Inclusion and Exclusion

Inclusion and Exclusion Criteria are as follows.

### Inclusion Criteria:

1. Must provide written or electronic consent after the nature of the study has been explained, and prior to any research-related procedures, per ICH GCP
2. Clinical diagnosis of PXE supported by prior genetic identification of biallelic ABCC6 mutations
3. Male or female, 18 to <65 years of age at Screening
4. Plasma PPi<1300 nM at Screening
5. Subjects who are being treated with statins or proprotein convertase subtilisin/kexin type 9 (PCSK9) inhibitors must be on stable doses for 3 years prior to enrollment through end of study unless the investigator deems, in consultation with the Sponsor, that the change will not confound interpretation of the study data
6. Women of child-bearing potential (WOCBP) must have a negative serum pregnancy test at Screening
7. WOCBP and partners of fertile males who are WOCBP must agree to use 1 highly effective form of contraception and a barrier method from at least 1 month before the first dose of INZ-701 through 30 days after last dose of INZ-701 (greater than 5 half-lives of INZ-701). WOCBP and partners of fertile males who are WOCBP must also agree to not donate ova from the period following the first dose of INZ-701 through 30 days after last dose of INZ-701.
8. Males who are sexually active must agree to use condoms from the period following first dose of INZ-701 through 30 days after the last dose of INZ-701. Males must also agree to not donate sperm from the period following the first dose of INZ-701 through 30 days after last dose of INZ-701.
9. In the opinion of the Investigator, must be willing and able to complete all aspects of the study
10. Agree to provide access to relevant medical records

### Exclusion Criteria:

1. In the opinion of the Investigator, presence of any clinically significant disease (outside of those considered associated with the diagnosis of ABCC6 Deficiency) that precludes study participation or may confound interpretation of study results, including known uncontrolled thyroid disease or unrelated connective tissue, bone, mineral, ophthalmologic, or muscle disease
2. Advanced eye disease requiring anti-VEGF treatment at Screening
3. Clinically significant abnormal laboratory result at Screening.
4. Screening laboratory results demonstrating elevations of aspartate aminotransferase, alanine aminotransferase, bilirubin (unless due to Gilbert disorder), eGFR>60,25-hydroxyvitamin D (25[OH]D) levels <20 ng/mL, parathyroid hormone (PTH)>40% above the upper limit of normal, or significant hyper- or hypocalcemia. Note: Rescreening for certain assessments is permitted as described in the protocol.
5. Known active fungal, bacterial, and/or viral infection including human immunodeficiency virus, hepatitis B virus, hepatitis C virus, or COVID-19 virus. A negative COVID-19 test result is required within 5 days prior to first dose of INZ-701.
6. Malignancy within the last 5 years, except non-melanoma skin cancers or cervical carcinoma in situ.
7. Known intolerance to INZ-701 or any of its excipients.
8. Unable or unwilling to discontinue the use of any prohibited medication (examples include bisphosphonates, calcimimetics, antacids, systemic corticosteroids, pyrophosphate containing medications) as provided in the protocol. Discontinuation should be undertaken only if considered not detrimental and indicated by the subject's treating physician.
9. Receipt of any other investigational new drug within 5 half-lives of the last dose of the other investigational product or from 4 weeks prior to the first dose of INZ-701, whichever is longer, or use of an investigational device, through completion of participation in the study
10. Last symptoms from a COVID-19 vaccination within 14 days prior to the first dose of INZ-701 or as described in Inozyme COVID-19 Vaccine Guidance Document.
11. Subjects who are pregnant, trying to become pregnant, or breastfeeding.
12. Subjects who are trying to father a child.

### Example 8: Treatment of a Patient Diagnosed with GACI Having ABCC6 Deficiency

ABCC6 deficiency may mask as GACI. GACI is a rare disease occurring in infants and involving extensive arterial calcification (Albright, et al., 2015, Nature Comm. 10006). GACI is believed to occur due to a defective ENPP1 protein. Surprisingly, in some instances, GACI phenotype is observed even when the subject has ABCC6 deficiency. Patients with ABCC6 deficiency can be identified by using isolated DNA samples using protocols described in J Med Genet. 2007 October; 44(10): 621-628. (Mutation detection in the ABCC6 gene and genotype—phenotype analysis in a large international case series affected by pseudoxanthoma elasticum, Ellen G Pfendner, et al)

ENPP1-Fc is administered at a selected dose of ENPP1-FC is one of 0.2 mg/kg, 0.6 mg/kg, or 1.8 mg/kg SC at least twice weekly for a period of time determined by the medical professional. GACI Patient (having ABCC6 deficiency) response to enzyme replacement is monitored as appropriate, as determined by the medical professional, e.g., by following a reduction in one or more symptoms of GACI, and/or using guidance provided herein.

The following examples, in the context of the entire specification, provide guidance to determine treatment protocols and efficacy.

### Example 9: Treatment of Patient Diagnosed with ARHR2 Having ABCC6 Deficiency

ABCC6 deficiency may mask as symptoms of ARHR2. ARHR2 is a rare skeletal disorder characterized by low levels of plasma PPi and serum phosphate which can result in rickets, repeated fractures of the long bones, rachitic skeletal deformities and impaired growth and development (Ferreira et al 2014, Moran 1975, Rutsch et al 2008). In some subjects, ARHR2 phenotype is observed when the subject has ABCC6 deficiency. Patients with ABCC6 deficiency can be identified by following the procedure in Example 4.

ENPP1-Fc is administered at a selected dose of ENPP1-FC is one of 0.2 mg/kg, 0.6 mg/kg, or 1.8 mg/kg SC at least twice weekly for a period of time determined by the medical professional. ARHR2 Patient response to enzyme replacement is monitored as appropriate, as determined by the medical professional, e.g., by following a reduction in one or more symptoms of ARHR2, using guidance provided herein.

The following examples, in the context of the entire specification, provide guidance to determine treatment protocols and efficacy.

### Example 10: Treatment of Patient Diagnosed with Chronic Kidney Disease (CKD)

Pathological calcification can manifest as another disease state referred as CKD. Clinical symptoms of chronic kidney diseases include itching, muscle cramps, nausea, lack of appetite, swelling of feet and ankles, sleeplessness and labored breathing. Chronic kidney disease if left untreated tends to progress into End stage renal disease (ESRD). (Chronic Kidney Disease Diagnosis and Management: A Review, Chen T K, Knicely D H, Grams M E. Chronic Kidney Disease Diagnosis and Management: A Review. JAMA. 2019 Oct. 1; 322(13):1294-1304.)

ENPP1-Fc is administered at a selected dose of ENPP1-FC is one of 0.2 mg/kg, 0.6 mg/kg, or 1.8 mg/kg SC at least twice weekly for a period of time determined by the medical professional. CKD Patient response to enzyme replacement is monitored as appropriate, as determined by the medical professional, e.g., by following a reduction in one or more symptoms of CKD, using guidance provided herein.

The following examples, in the context of the entire specification, provide guidance to determine treatment protocols and efficacy.

### Example 11: Treatment of Patient Diagnosed with Ossification of Posterior Longitudinal ligament (OPLL)

Pathological calcification can manifest as another disease state referred as OPLL. Clinical symptoms and signs caused by OPLL are categorized as: (1) myelopathy, or a spinal cord lesion with motor and sensory disturbance of the upper and lower limbs, spasticity, and bladder dysfunction; (2) cervical radiculopathy, with pain and sensory disturbance of the upper limbs; and (3) axial discomfort, with pain and stiffness around the neck. The most common symptoms in the early stages of OPLL include dysesthesia and tingling sensation in hands, and clumsiness. (Wu J C, Chen Y C, Huang W C. Ossification of the Posterior Longitudinal Ligament in Cervical Spine: Prevalence, Management, and Prognosis. Neurospine. 2018 March; 15(1):33-41.)

ENPP1-Fc is administered at a selected dose of ENPP1-FC is one of 0.2 mg/kg, 0.6 mg/kg, or 1.8 mg/kg SC at least twice weekly for a period of time determined by the medical professional. OPLL Patient response to enzyme replacement is monitored as appropriate, as determined by the medical professional, e.g., by following a reduction in one or more symptoms of OPLL, using guidance provided herein.

The following examples, in the context of the entire specification, provide guidance to determine treatment protocols and efficacy.

### Example 12: Treatment of Patient Diagnosed with Hereditary Hypophosphatemic Rickets

Pathological calcification can manifest as another disease state referred as Hereditary Hypophosphatemic Rickets (HHR). Clinical symptoms and signs caused by HHR are categorized as: (1) myelopathy, or a spinal cord lesion with motor and sensory disturbance of the upper and lower limbs, spasticity, and bladder dysfunction; (2) cervical radiculopathy, with pain and sensory disturbance of the upper limbs; and (3) axial discomfort, with pain and stiffness around the neck. The most common symptoms of HHR include premature fusion of the skull bones (craniosynostosis) and dental abnormalities. The disorder may also cause abnormal bone growth where ligaments and tendons attach to joints (enthesopathy). In adults, hypophosphatemia is characterized by a softening of the bones known as osteomalacia. (Cho H Y, Lee B H, Kang J H, Ha I S, Cheong H I, Choi Y. A clinical and molecular genetic study of hypophosphatemic rickets in children. Pediatr Res. 2005 August; 58(2):329-33.)

ENPP1-Fc is administered at a selected dose of ENPP1-FC is one of 0.2 mg/kg, 0.6 mg/kg, or 1.8 mg/kg SC at least twice weekly for a period of time determined by the medical professional. HHR Patient response to enzyme replacement is monitored as appropriate, as determined by the medical professional, e.g., by following a reduction in one or more symptoms of HHR, using guidance provided herein.

The following examples, in the context of the entire specification, provide guidance to determine treatment protocols and efficacy.

### Example 13: Treatment of Patient Diagnosed with X-Linked Hypophosphatemia (XLH)

Pathological calcification can manifest as another disease state referred as X-linked hypophosphatemia (XLH). X-linked dominant hypophosphatemic rickets, or X-linked Vitamin D-resistant rickets, is an X-linked dominant form of rickets (or osteomalacia) that differs from most cases of rickets in that vitamin D supplementation does not cure it. It can cause bone deformity including short stature and genu varum (bow leggedness). It is associated with a mutation in the PHEX gene sequence (Xp.22) and subsequent inactivity of the PHEX protein. (Carpenter T O, Imel E A, Holm I A, Jan de Beur S M, Insogna K L. A clinician's guide to X-linked hypophosphatemia. J Bone Miner Res. 2011 July; 26(7):1381-8. doi: 10.10021jbmr.340. Epub 2011 May 2. Erratum in: J Bone Miner Res. 2015 February; 30(2):394)

ENPP1-Fc is administered at a selected dose of ENPP1-FC is one of 0.2 mg/kg, 0.6 mg/kg, or 1.8 mg/kg SC at least twice weekly for a period of time determined by the medical professional. XLH Patient response to enzyme replacement is monitored as appropriate, as determined by the medical professional, e.g., by following a reduction in one or more symptoms of XLH, using guidance provided herein.

The following examples, in the context of the entire specification, provide guidance to determine treatment protocols and efficacy.

### Example 14: Treatment of Patient Diagnosed with Autosomal Dominant Hypophosphatemic Rickets (ADHR)

Pathological calcification can manifest as another disease state referred as Autosomal Dominant Hypophosphatemic Rickets (ADHR). Clinical symptoms and signs caused by ADHR poorly formed bones (rickets), bone pain, and tooth abscesses. ADHR is caused by a mutation in the fibroblast growth factor 23 (FGF23). ADHR is characterized by impaired mineralization of bone, rickets and/or osteomalacia, suppressed levels of calcitriol (1,25-dihydroxyvitamin D3), renal phosphate wasting, and low serum phosphate. Mutations in FGF23 render the protein more stable and uncleavable by proteases resulting in enhanced bioactivity of FGF23. The enhanced activity of FGF23 mutants reduce expression of sodium-phosphate co-transporters, NPT2a and NPT2c, on the apical surface of proximal renal tubule cells, resulting in renal phosphate wasting. (Rowe P S. The wrickkened pathways of FGF23, MEPE and PHEX Crit Rev Oral Biol Med. 2004 Sep. 1; 15(5):264-81.)

ENPP1-Fc is administered at a selected dose of ENPP1-FC is one of 0.2 mg/kg, 0.6 mg/kg, or 1.8 mg/kg SC at least twice weekly for a period of time determined by the medical professional. ADHR Patient response to enzyme replacement is monitored as appropriate, as determined by the medical professional, e.g., by following a reduction in one or more symptoms of ADHR, using guidance provided herein.

The following examples, in the context of the entire specification, provide guidance to determine treatment protocols and efficacy.

### Example 15: Measurement of Plasma Inorganic Pyrophosphate

Low plasma PPi levels are a characteristic of ENPP1 Deficiency and are used as an indicator of treatment efficacy. ENPP1-Fc specifically cleaves ATP to generate AMP and PPi. The therapeutic goal of ENPP1 ERT is to normalize extracellular PPi levels and correct clinical abnormalities associated with ENPP1 Deficiency.

PPi is measured by obtaining patient plasma samples. Determined PPi data may be used to adjust dose levels. PPi levels may also serve as the primary PD marker for PK/PD analysis.

The concentration of Pi and PPi in mammals is 1-3 mM and 2-3 μM respectively.

### Example 16: Biomarkers Associated with Bone Health

In addition to low plasma PPi, ENPP1 deficient patients are characterized biochemically by low serum phosphate, high urine phosphate, low renal TmP/GFR, normal calcium (Ca), low-normal urine Ca, normal 25-hydroxy Vitamin D (25 OH D), low-normal 1.25(OH)2D, high BAP, high intact FGF23, and normal PTH (IOF 2019).

Biomarkers that may be used as additional determinants of bone health of a treated patient are set forth in Table 3.

TABLE 3
Clinical Intermediates and Biomarkers

| | Laboratory Analyte | Sample Type |
|---|---|---|
| Primary | Pyrophosphate (PPi) | plasma |
| Pharmacodynamic | Inorganic phosphate | serum |
| Markers | FGF23 (intact) | Plasma |
| | TmP/GFR | serum creatinine, |
| | | serum phosphate, |
| | ALP, BALP, | urine phosphate |
| | CTx, P1NP | serum, plasma, urine |

### Example 17: Efficacy of Treatment with ENPP1-Fc

Treatment efficacy may be assessed by measuring plasma PPi as well as measuring other plasma analytes, such as FGF23, Pi, FGF23, Pi, TmP/GFR, serum alkaline phosphatase (ALP), bone-specific ALP (BALP), carboxy terminal cross-linked telopeptide of type I collagen (CTx), and procollagen type 1 N-terminal propeptide (P1NP). These analyte measurements may be used as a PD markers associated with ENPP1 Deficiency to determine the efficacy for ENPP1-FC. Changes in these analytes may be described as changes from baseline and in a time-dependent manner over the course of treatment. Dose linearity of PK and PD parameters also may be assessed.

Changes from baseline in plasma PPi levels, FGF23 levels and Urinary phosphorus excretion per creatinine clearance may be analyzed using a t test of paired differences to test the null hypothesis that

### Example 18: Drug Concentration Measurements

In addition, blood samples may be obtained from a patient for measurement of ENPP1-FC concentration in plasma and subsequent determination of PK parameters following the first dose (i.e. single dose) and at/after multiple doses (i.e. steady-state).

### Example 19: Immunogenicity (Anti-Drug Antibodies)

If desired, immunogenicity to ENPP1-Fc may be measured using anti-drug antibodies (ADA). Immunogenicity testing can utilize a multi-tiered approach; if ADA are detected in the initial screen, a confirmatory test may be run to determine specificity. Samples may also be used to assess and further establish assays for specificity confirmation (i.e. titer) and neutralizing antibodies.

### Example 20: Pharmacokinetic, Pharmacodynamic, and Exploratory Biomarker Analyses

Pharmacokinetic analysis may be performed on the PK population, and PK parameters of ENPP1-FC may be summarized by treatment with descriptive statistics. Dose linearity of PK and PD parameters may also be assessed. PK/PD analyses, immunogenicity analyses; and exploratory biomarker analyses may be determined.

### Example 21: Additional Determinators of Efficacy

Although restoring a normal level of PPi is the primary indicator of efficacy of treatment using NEPP1-Fc, other physical measurements also may be used, if desired to assist in determining treatment efficacy. These include one or more of the following.

### 1. Radiography and Imaging

X-Rays for Skeletal Severity. Standard X-rays may be obtained to detect rachitic skeletal deformities. Obtain X-rays may be obtained, for example, on the wrists and knees.

DEXA Scan. DEXA scans may be used to evaluate changes in bone density.

Positron Emission Tomography—Computed Tomography. Baseline Na¹⁸F-PET/HRpQCT (or HR-CT) may be a full body scan done within 1 month of first dose of ENPP1-FC to measure calcification of arteries and organs and skeletal abnormalities at baseline and for future interventional assessments. The Na¹⁸F-PET measures bone turnover as well as microcalcification of the arteries. High-resolution quantitative computed tomography (HRQCT) or HR-CT can determine bone microstructure at the non-dominant distal radius and tibia. Standard bone geometric parameters are calculated.

Doppler Echocardiogram. A baseline echocardiogram may be obtained within 3 days prior to a first dose of ENPP1-FC. Doppler echo may be used to measure heart function [LVEF, blood flow] calcification of heart and valves, and arterial stiffness.

Optical Coherence Tomography. Optical coherence tomography may be used to visualize neointimal proliferation.

Peripheral Arterial Tonometry. Peripheral arterial tonometry (PAT) may be used to assess digital pulse wave amplitude (PWA), which corresponds to digital volume variation.

Renal Ultrasound. Renal ultrasound may be used, for example, within 1 week of starting ENPP1-FC, to measure renal calcification.

Bone Histomorphology and Bone Biopsy. Bone biopsy may be performed as a baseline measurement. Tetracycline loading for 10 days prior to bone biopsy is preferred. 2. Walking Ability. Walk tests may be used as a submaximal exercise measurement to measure functional capacity in ambulatory patients combining cardiopulmonary, neuromuscular, and musculoskeletal functions. The 6 Minute Walk Test (6 MWT) was originally developed by the American Thoracic Society (ATS 2002) for use with adults, and is now commonly used in both adult and pediatric populations (Mylius et al 2016), and with children with neuromuscular diseases such as spinal muscular atrophy (Montes et al 2018), Duchenne muscular dystrophy (McDonald et al 2013), and infantile-onset Pompe disease (van der Meijden et al 2018). The 2 Minute Walk Test (2 MWT) is included in the NIH Toolbox and is increasingly being used to measure the same properties.

The 6 MWT and the 2 MWT may be administered to the patient before and during treatment at the discretion of the healthcare provider. If a subject is unable to complete at least the 2 MWT at baseline, additional assessments during treatment may be left to a healthcare provider's discretion. Resting heart rate is obtained prior to the test and post-test. Distance walked in the first 2 minutes of the 6 MWT and the full 6 minutes may be recorded. The distances walked in 2 minutes and 6 minutes may be compared to age- and sex-matched normative data (percent predicted values).

3. Dynamometry. Strength may be assessed using dynamometry before and/or during treatment at the discretion of the healthcare provider. Hand-held dynamometry is a direct measurement of strength commonly used in both children and adults. Muscle groups that may be assessed include: shoulder abduction, shoulder flexion, elbow flexion, elbow extension, hip abduction, hip flexion, hip extension, and knee extension. Each muscle group may be measured 2 times bilaterally.

Grip Strength. Grip strength may be measured using a grip strength dynamometer before and/or during treatment at the discretion of the healthcare provider. Equipment and assessor instructions may be standardized across sites. Grip may be assessed bilaterally with 1 practice and 1 maximal force measures taken for each hand and results may be compared to age and gender matched normative data (when available).

Range of Motion. Range of Motion may be assessed using a goniometer, an instrument that tests the angle of joints and measures the degree of movement at a joint. The stationary arm of the goniometer is aligned with the specified bony landmark on the stationary body segment, and the moving arm of the goniometer is aligned with the specified bony landmark of the limb that is moving. The fulcrum of the goniometer is specified for each motion measured using axis of motion and bony landmarks. Range of motion may be assessed for one or more of the following: shoulder abduction, shoulder flexion, elbow flexion, elbow extension, hip abduction, hip flexion, hip extension, and knee extension.

4. Hearing Testing. Moderate hearing loss has been associated with ARHR2 (Brachet et al 2014, Steichen-Gersdorf et al 2015). Baseline hearing may be determined by one or more of: Physical exam and otoscopy, Immittance audiometry (commonly called tympanometry), Pure Tone Audiometry (PTA) with frequencies up to 8 kHz if possible. (If there is a PTA threshold of >15 dB, the subject should also undergo bone conduction testing), High Frequency Audiometry (HFA), with frequencies up to 16 kHz.

5. Clinician Global Impression Scales. The Clinical Global Impression (CGI-S) scales were developed for use in National Institute of Mental Health-sponsored clinical studies to provide a brief, stand-alone assessment of the clinician's view of the patient's global functioning prior to and after initiating a study medication (Guy 1976). The CGI provides an overall clinician-determined summary measure that considers all available information, including knowledge of the patient's history, psychosocial circumstances, symptoms, behavior, and the impact of the symptoms on the patient's ability to function. The CGI-S may be administered before and/or during treatment at the discretion of the healthcare provider and provides a global assessment of change using a seven-point scale ranging from −3 (severe worsening) to +3 (significant improvement). The Table below shows the scale and the corresponding impression which is an indicator of overall health of the patient when compared to a base line such as that of a healthy person or health of a patient prior to dosage regimen. (See Guy, W. Clinical global impressions (CGI) scale. Handbook of Psychiatric Measures. Washington, DC: American Psychiatric Association. 2000:100-102)

| Unit | Impression |
|---|---|
| −3 | Very much worse than baseline |
| −2 | Much worse than baseline |
| −1 | Minimally worse than baseline |
| 0 | No change compared to baseline |
| 1 | Minimally improved compared to baseline |
| 2 | Much improved compared to baseline |
| 3 | Very much improved compared to baseline |

6. Gross Motor Function Classification System—Expanded and Revised. The Gross Motor Classification System—Expanded and Revised (GMFCS—E and R) may be administered before and/or during treatment at the discretion of the healthcare provider. The GMFCS—E and R classifies patient-initiated movement with an emphasis on mobility on a scale from 1 to 5.

7. Patient Reported Outcomes Measurement Information Systems. The Patient Reported Outcomes Measurement Information Systems (PROMIS) consists of a variety of questionnaires developed by the National Institutes of Health (NIH) to evaluate physical, mental, and social well-being from the patient perspective (http://www.healthmeasures.net). These questionnaires have been used in clinical studies in people with chronic health conditions such as X-linked hypophosphatemia, arthritis, multiple sclerosis, and neurofibromatosis. Each questionnaire contains 8 to 10 items which are rated by the participant on a 5-point Likert scale ranging from 1 (never) to 5 (always). Scores are summed for each questionnaire, with high scores indicating more of the domain being measured (e.g. more fatigue, more physical function). Raw scores are converted to T-Scores based on a mean of 50 and a standard deviation of 10, allowing comparison of the study sample to the general population. PROMIS Scales may include the Pain Interference (short form 8a), Pain Intensity (version 3a), Physical Function—Upper Extremity (custom short form), Physical Function—Mobility (short form 13a FACIT Fatigue), Fatigue (short form), and Cognitive Impact (short form 8a) and may be administered before and/or during treatment at the discretion of the healthcare provider. These assessments may be completed by the subject without assistance.

8. Caregiver Global Impression Scales. The Caregiver Global Impression of Status may be administered to the patient's caregiver before and/or during treatment at the discretion of the healthcare provider. The Caregiver Global Impression of Change provides a global assessment of change using a seven-point scale ranging from −3 (severe worsening) to +3 (significant improvement).

9. Western Ontario and McMaster University Osteoarthritis Index. The WOMAC is a patient-reported outcome used to assess activities of daily living, functional mobility, gait, general health, pain, and quality of life in patients with hip or knee pain (www.sralab.org). The assessment consists of 24-items and takes approximately 12 minutes to administer. The WOMAC may be administered before and/or during treatment at the discretion of the healthcare provider. The assessment may be completed by the subject without assistance.

### Example 22: Treatment of ENPP1 Deficient Patient with Low Dose of ENPP1-Fc

ENPP1-Fc was administered subcutaneously (SC) at 0.2 mg/kg to a cohort of 3 patients who suffer from ENPP1 deficiency. The first dose of ENPP1-FC was administered on Day 1. On Days 8 and thereafter, ENPP1-Fc is administered to a subject at a selected dose of ENPP1 agent 0.2 mg/kg dose twice weekly for a study period of 32 days. The dose was administered at approximately the same time on each dosing day. The site of injection was alternated, with no site within 2 inches of any prior site of injection within the prior 2 weeks.

Blood samples were collected from each patient prior to administration of ENPP1-Fc on Day 0 to establish a baseline for PPi levels, and ENPP1 activity. Samples were also collected from each patient after the dose from Days 1-32. Each Patient's response to enzyme replacement was monitored as appropriate, as determined by the medical professional, e.g., by following a reduction in one or more symptoms of ENPP1 deficiency, and/or using guidance provided herein.

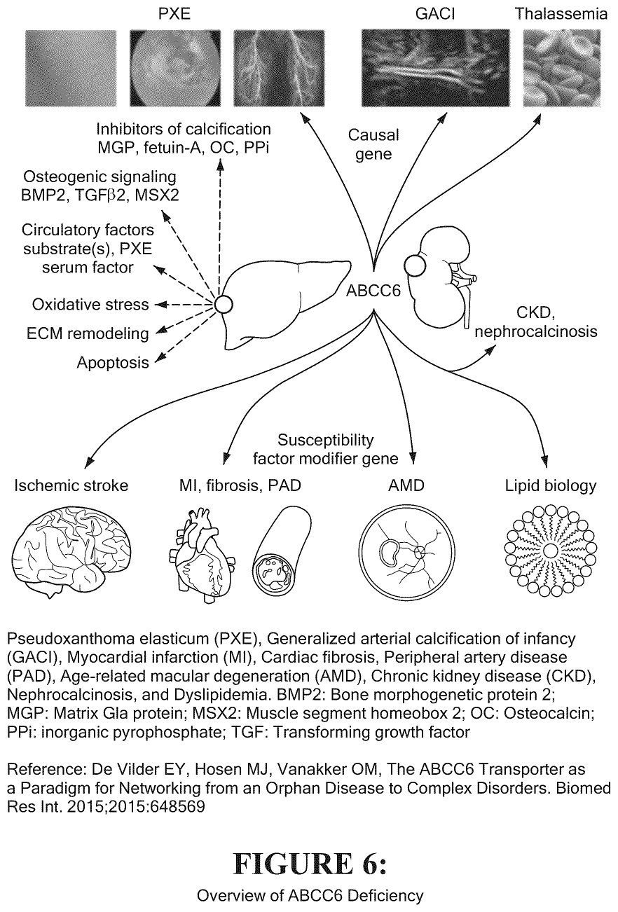

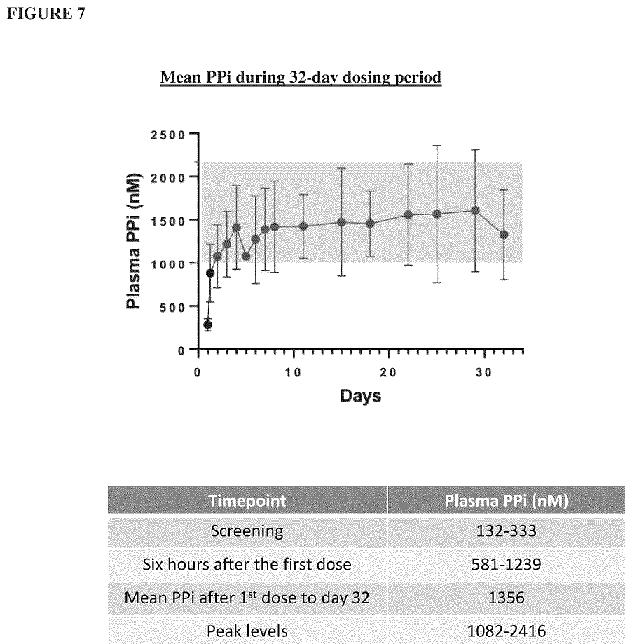

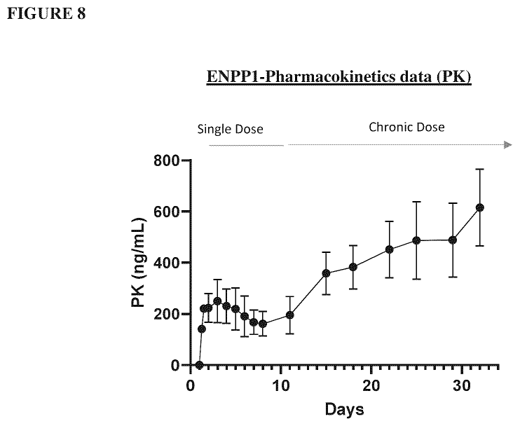

Ppi levels, ENPP1 activity levels, and PK value of ENPP1 were determined for each sample collected. The results of the dose study are shown in FIGS. 7-9. FIG. 7 shows PPi levels before and after administration of ENPP1-Fc at 0.2 mg/kg from Day 0 to Day 31. Likewise FIGS. 8 and 9 show the measured ENPP1 activity and PK data for samples collected from Day 0 to 31.

The results show a rapid, significant, and sustained increase in PPi levels observed in all three adult patients dosed with ENPP1. (See FIG. 7). The PPi levels increased in all patients to levels comparable to those observed in healthy subjects (˜1.5-3 μM). The Table shows the peak and median value of the PPi levels observed.

The PK data indicated that the ENPP1 activity level was approaching a steady-state by Day 29 and exhibited nearly 4-fold accumulation when compared with the levels of ENPP1 on Day 1. The PK data and ENPP1 activity data were consistent with each other amongst all three patients. (See FIGS. 8 & 9).

Analysis of the patients also indicated that the ENPP1-Fc administration was generally well-tolerated and exhibited a favorable initial safety profile. No serious adverse events (SAEs) were observed in any of the patients. No injection site reactions were observed.

The patients were continuously monitored after the 32-day dose period and at Week 12, low titers of anti-drug antibodies (ADAs) were observed in two out of three patients. However, the increased PPi levels observed during the 32-day dose evaluation period were still sustained in all three patients through Week 12.

The presence of sustained ENPP1 activity and increase in ENPP1 protein levels indicate that once-weekly dosing is sufficient to produce the desired treatment outcome.

### Example 23: Treatment of ENPP1 Deficient Patients with Different Doses of ENPP1-Fc for an Extended Phase H Study Period

ENPP1-Fc was administered subcutaneously (SC) at 0.2 mg/kg to a cohort (1) of 3 patients, at 0.6 mg/kg to cohort (2) of 3 patients and 1.8 mg/kg to another cohort (3) of 3 patients, all of whom suffer from ENPP1 deficiency. The first dose of ENPP1-Fc was administered on Day 1.

On Days 8 and thereafter, ENPP1-Fc is administered to a subject at a selected dose of ENPP1 agent 0.2 mg/kg dose twice weekly to cohort (1) patients for a phase I study period of 32 days. Similarly, on days 8 and thereafter, ENPP1-Fc is administered to a subject at a selected dose of ENPP1 agent 0.6 mg/kg dose twice weekly to cohort (2) patients for a phase I study period of 32 days. Likewise, on days 8 and thereafter, ENPP1-Fc is administered to a subject at a selected dose of ENPP1 agent 1.8 mg/kg dose twice weekly to cohort (3) patients for a phase I study period of 32 days.

After day 32, subjects from each cohorts 1, 2 & 3 continued receiving the same dose (0.2 mg/kg, 0.6 mg/kg, 1.8 mg/kg respectively) that was assigned in the phase I study period for an extended period of 48 weeks (phase II study period of 336 days). The dose was administered at approximately the same time on each dosing day. The site of injection was alternated, with no site within 2 inches of any prior site of injection within the prior 2 weeks.

Blood samples were collected from each patient prior to administration of ENPP1-Fc on Day 0 to establish a baseline for PPi levels, and ENPP1 activity. Blood samples were also collected from healthy volunteers (HV) for determining normal PPi levels, and normal ENPP1 activity in subjects without ENPP1 deficiency.

Samples were also collected from each patient after the dose from Days 1-32 (Phase I). Samples were also collected from each patient after the dose from Days 32-338 (Phase II). Each Patient's response to enzyme replacement was monitored as appropriate, as determined by the medical professional, e.g., by following a reduction in one or more symptoms of ENPP1 deficiency, and/or using guidance provided herein.

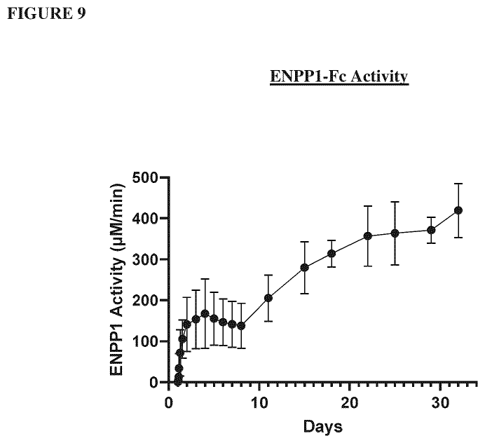

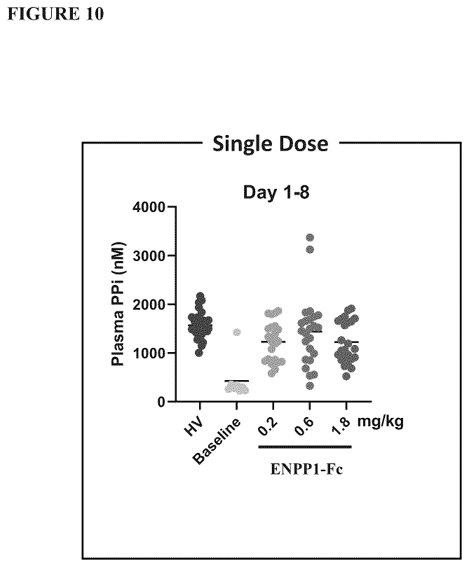

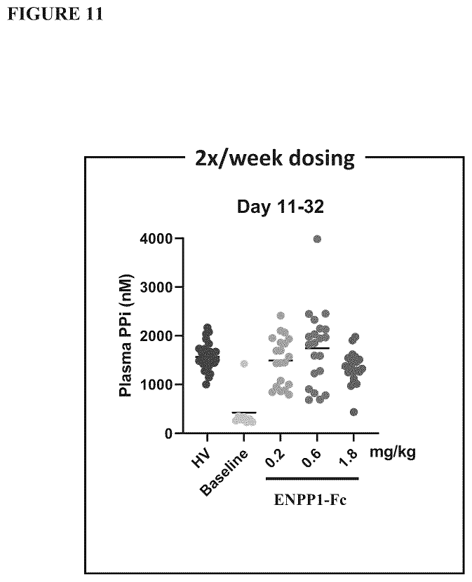

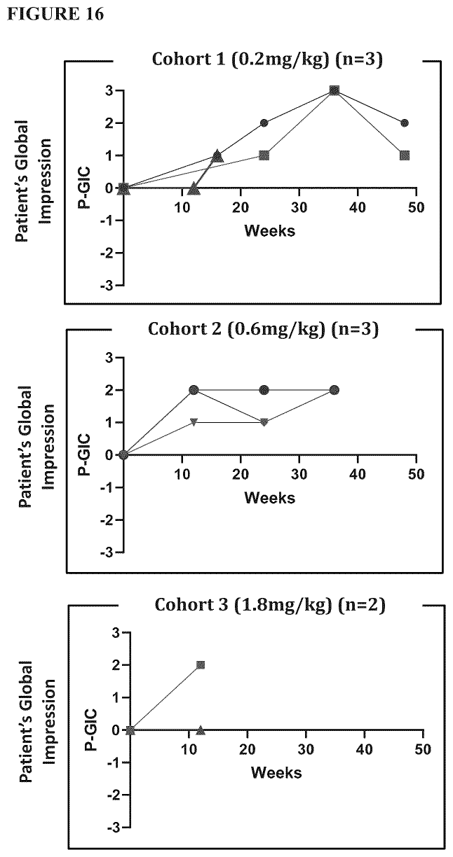

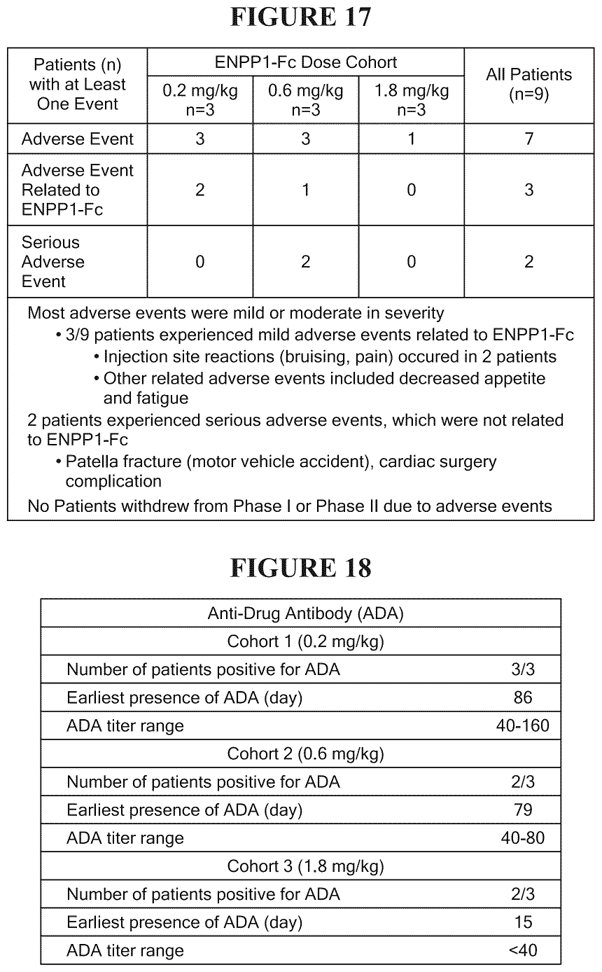

Ppi levels, ENPP1 activity levels, and PK value of ENPP1 were determined for each sample collected. The results of the administered dosage to patient cohorts for an extended study period are shown in FIGS. 10-18.

The results show a rapid, significant, and sustained increase in PPi levels observed in all three adult patients (Cohort 1) dosed with 0.2 mg/kg of ENPP1. (See FIGS. 10-12). Similar results of significant, and sustained increase in PPi levels was observed in patients dosed with 0.6 mg/ml (Cohort 2) and also in patients dosed with 1.85 mg/ml (Cohort 3). The PPi levels increased in all patients to levels comparable to those observed in healthy subjects (˜1.5-3 μM). Rapid increase in PPi levels was observed after the first dose in all cohorts.

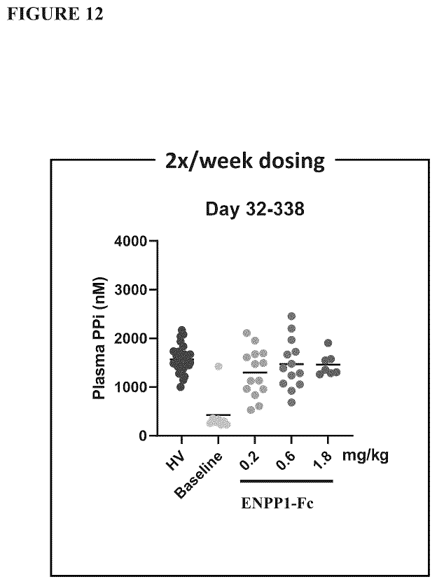

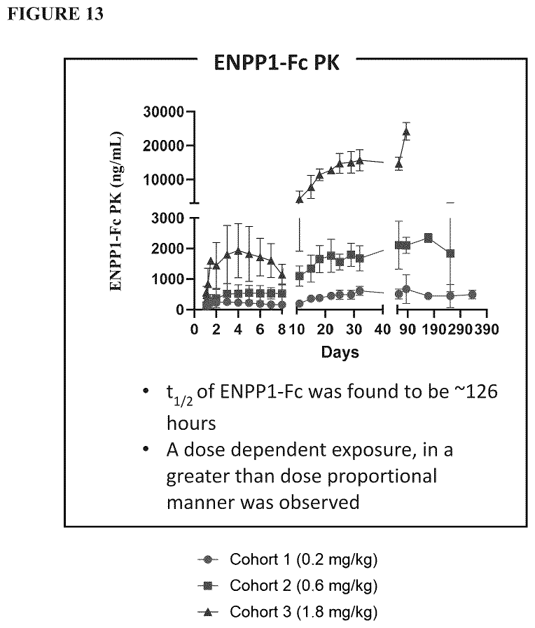

Likewise FIGS. 13 and 14 show the measured ENPP1 activity and pharmacokinetics (PK) data for samples collected from Day 0 to 338. The results showed that ENPP1-Fc had a half-life of over 126 hours.

The PK data indicated that the ENPP1 activity level was approaching a steady-state by Day 29 and exhibited nearly 4-fold accumulation when compared with the levels of ENPP1 on Day 1. The PK data and ENPP1 activity data were consistent with each other in all three cohorts of dosages (0.2 mg/kg, 0.6 mg/kg & 1.8 mg/kg) (See FIGS. 13 & 14).

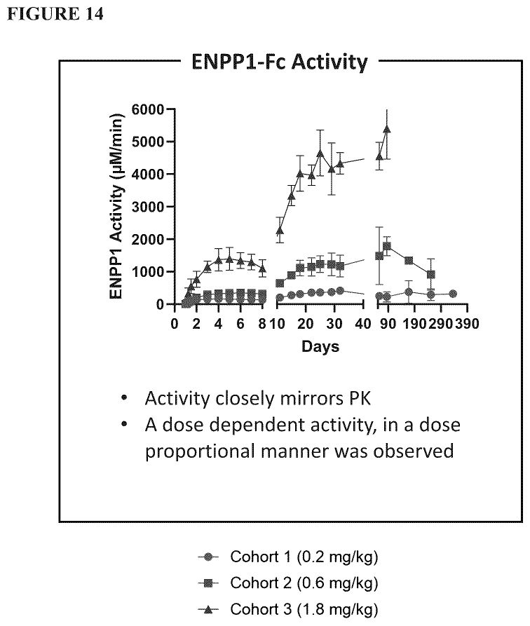

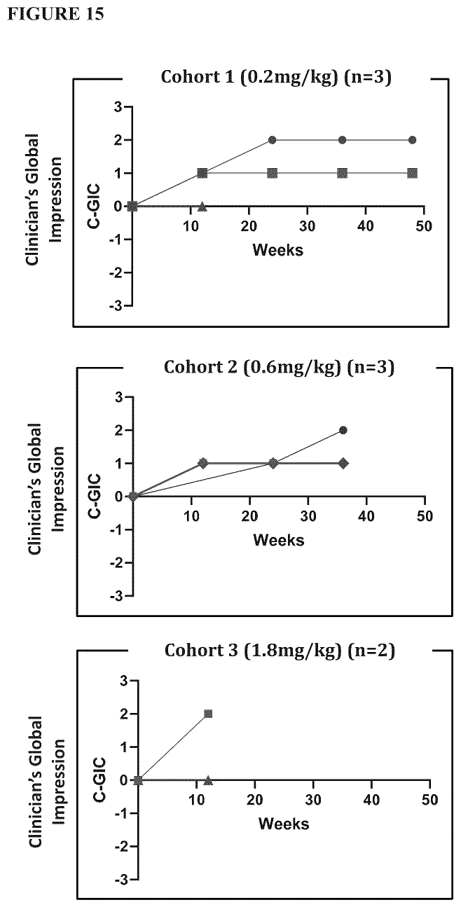

FIGS. 15 and 16 shows the Clinician's global impression scale and Patient's global impression scale for each of the three dosing cohorts. The Global impression of change scale (GIC) ranges from −3 to +3 with each unit signifying the patient's or clinician's impression of how the treatment is progressing and improvement in disease state is occurring. In all three cohorts (1, 2 & 3) who were exposed to different doses (0.2 mg/kg, 0.6 mg/kg & 1.8 mg/kg respectively) there was a strong concordance in improved clinician's and patient's global impression of change.

Analysis of the patients also indicated that the ENPP1-Fc administration was generally well-tolerated and exhibited a favorable initial safety profile. No serious adverse events (SAEs) were observed in any of the patients in any of the cohorts being treated with 0.2 mg/kg, 0.6 mg/kg and 1.8 mg/kg respectively. Minor injection site reactions such as bruising, and pain were observed but none were severe.

The patients were continuously monitored after the 32-day dose period and at Week 48, low titers of anti-drug antibodies (ADAs) were observed in two out of three patients. However, the increased PPi levels observed during the 32-day dose evaluation period were still sustained in all three patients through Week 48. The overall results of the extended study period indicate that ENPP1-Fc administration was generally well-tolerated and exhibited a favorable safety profile.

The presence of sustained ENPP1 activity, increased PPi levels and increased in ENPP1 protein levels over an extended study period of 48 weeks indicate that once-weekly dosing is sufficient to produce the desired treatment outcome.

### REFERENCES

ATS. American Thoracic Society Statement: Guidelines for the six-minute walk test. AmJRespir, CritCare Med. 2002 2002; 166(1):111-7.
Brachet C, Mansbach A L, Clerckx A, Deltenre P, Heinrichs C. Hearing loss is part of the clinical picture of ENPP1 loss of function mutation. Horm Res Paediatr. 2014; 81(1):63-6.
CTFG. Recommendations related to contraception and pregnancy testing in clinical trials. 2014 [19 May 2020]; Available from:
http://www.hma.eu/fileadmin/dateien/Human_Medicines/01-About_HMA/Working Groups/CTFG/2014 09 HMA CTFG Contraception.pdf.
Ferreira C, Ziegler S, Gahl W. Generalized Arterial Calcification of Infancy. In: Adam M P, Ardinger H H, Pagon R A, Wallace S E, Bean L J H, Stephens K, et al., editors. GeneReviews((R)). Seattle (WA)2014.
Guy W. The Clinical Global Impression Scale. In: Rush Jr A J, First M B, Blacker D, editors. Handbook of Psychiatric Measures, 2008. Washington, D C: American Psychiatric Publishing, Inc; 1976. p. 90-2.
IOF. Autosomal Recessive Hypophosphatemic Rickets Type 2 (ARHR2). 2019 [19 May 2020]; Available from: https://www.iofbonehealth.org/osteoporosis-musculoskeletal-disorders/skeletal-rare-disorders/autosomal-recessive-hypophosphatemi-0.
Mackenzie N C, Huesa C, Rutsch F, MacRae V E. New insights into NPP1 function: lessons from clinical and animal studies. Bone. 2012 November; 51(5):961-8.
McDonald C M, Henricson E K, Abresch R T, Florence J M, Eagle M, Gappmaier E, et al. The 6-minute walk test and other endpoints in Duchenne muscular dystrophy: longitudinal natural history observations over 48 weeks from a multicenter study. Muscle Nerve. 2013 September; 48(3):343-56.
Montes J, McDermott M P, Mirek E, Mazzone E S, Main M, Glanzman A M, et al. Ambulatory function in spinal muscular atrophy: Age-related patterns of progression. PLoS One. 2018; 13(6):e0199657.
Moran J J. Idiopathic arterial calcification of infancy: a clinicopathologic study. Pathol Annu. 1975; 10:393-417.
Mylius C F, Paap D, Takken T. Reference value for the 6-minute walk test in children and adolescents: a systematic review. Expert Rev Respir Med. 2016 December; 10(12):1335-52.
NCI. National Cancer Institute Division of Cancer Treatment and Diagnosis (DCTD), National Cancer Institute (NCI), National Institutes of Health (NIH), Department of Health and Human Services (DHHS). Common Terminology Criteria for Adverse Events V5.0 (CTCAE). Published: Nov. 27, 2017. Available at https://ctep.cancer.gov/protocoldevelopment/electronic applications/docs/ctcae_v5_quick_reference_8.5x11.pdf. 2017.
Orriss I R, Arnett T R, Russell R G. Pyrophosphate: a key inhibitor of mineralisation. Curr Opin Pharmacol. 2016 June; 28:57-68.
Rutsch F, Boyer P, Nitschke Y, Ruf N, Lorenz-Depierieux B, Wittkampf T, et al. Hypophosphatemia, hypophosphaturia, and bisphosphonate treatment are associated with survival beyond infancy in generalized arterial calcification of infancy. Circ Cardiovasc Genet. 2008 December; 1(2):133-40.
Steichen-Gersdorf E, Lorenz-Depiereux B, Strom T M, Shaw N J. Early onset hearing loss in autosomal recessive hypophosphatemic rickets caused by loss of function mutation in ENPP1. J Pediatr Endocrinol Metab. 2015 July; 28(7-8):967-70.
van der Meijden J C, Kruijshaar M E, Harlaar L, Rizopoulos D, van der Beek N, van der Ploeg A T. Long-term follow-up of 17 patients with childhood Pompe disease treated with enzyme replacement therapy. J Inherit Metab Dis. 2018 November; 41(6):1205-14.

### Additional Embodiments

1 A method for preventing the progression of or reducing vascular calcification in a subject with ENPP1 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce vascular calcification in the subject.
2. A method for preventing the progression of or reducing pathological calcification in a subject with ENPP1 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce pathological calcification in the subject.
3. A method for preventing the progression of or reducing tissue calcification in a subject with ENPP1 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject
4. A method for preventing the progression of or reducing pathological ossification in a subject with ENPP1 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject.
5. A method for increasing circulating pyrophosphate (PPi) in a subject with ENPP1 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase circulating PPi in the subject.
6. A method for increasing pyrophosphatase activity in a subject with ENPP1 Deficiency, the method comprising: administering to the subject an ENPP1 agent at a dose of about 0.2 mg per kilogram of the subject, or about 0.4 mg per kilogram of the subject, about 0.6 mg per kilogram of the subject, or about 1.2 mg per kilogram of the subject, or about 1.8 mg per kilogram of the subject, to thereby increase circulating PPi in the subject.
7. A method for ameliorating one or more symptoms of ENPP1 Deficiency in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby ameliorate one or more symptoms of ENPP1 Deficiency in the subject.
8. A method for treating a subject with ENPP1 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat the subject.
9. A method for preventing the progression of or reducing vascular calcification in a subject with pathological calcification, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce vascular calcification in the subject.
10. A method for preventing the progression of or reducing pathological calcification in a subject with pathological calcification, the method comprising: administering to the subject an ENPP1 agent at a dose of about 0.2 mg per kilogram of the subject, or about 0.4 mg per kilogram of the subject, about 0.6 mg per kilogram of the subject, or about 1.2 mg per kilogram of the subject, or about 1.8 mg per kilogram of the subject, to thereby prevent the progression of or reduce pathological calcification in the subject.
11. A method for preventing the progression of or reducing tissue calcification in a subject with pathological calcification, the method comprising: administering to the subject an ENPP1 agent at a dose of about 0.2 mg per kilogram of the subject, or about 0.4 mg per kilogram of the subject, about 0.6 mg per kilogram of the subject, or about 1.2 mg per kilogram of the subject, or about 1.8 mg per kilogram of the subject, to thereby prevent the progression of or reduce tissue calcification in the subject.
12. A method for preventing the progression of or reducing pathological ossification in a subject, the method comprising: administering to the subject an ENPP1 agent at a dose of about 0.2 mg per kilogram of the subject, or about 0.4 mg per kilogram of the subject, about 0.6 mg per kilogram of the subject, or about 1.2 mg per kilogram of the subject, or about 1.8 mg per kilogram of the subject, to thereby prevent the progression of or reduce tissue calcification in the subject.
13. A method for increasing circulating pyrophosphate (PPi) in a subject with pathological calcification, the method comprising: administering to the subject an ENPP1 agent at a dose of about 0.2 mg per kilogram of the subject, or about 0.4 mg per kilogram of the subject, about 0.6 mg per kilogram of the subject, or about 1.2 mg per kilogram of the subject, or about 1.8 mg per kilogram of the subject, to thereby increase circulating PPi in the subject.
14. A method for increasing pyrophosphatase activity in a subject with pathological calcification, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase circulating PPi in the subject.
15. A method for ameliorating one or more symptoms of pathological calcification in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby ameliorate one or more symptoms of pathological calcification in the subject.
16. A method for treating a subject with pathological calcification, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat the subject.
17. A method for preventing the progression of or reducing vascular calcification in a subject with ABCC6 Deficiency or ABCC6 deficient subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce vascular calcification in the subject.
18. A method for preventing the progression of or reducing pathological calcification in a subject with ABCC6 Deficiency or ABCC6 deficient subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce pathological calcification in the subject.
19. A method for preventing the progression of or reducing tissue calcification in a subject with ABCC6 Deficiency or ABCC6 deficient subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby prevent the progression of or reduce tissue calcification in the subject
20. A method for preventing the progression of or reducing pathological ossification in a subject with ABCC6 Deficiency or ABCC6 deficient subject, the method comprising: administering to the subject an ENPP1 agent at a dose of about 0.2 mg per kilogram of the subject, or about 0.4 mg per kilogram of the subject, about 0.6 mg per kilogram of the subject, or or about 1.2 mg per kilogram of the subject, or about 1.8 mg per kilogram of the subject, to thereby prevent the progression of or reduce pathological ossification in the subject.
21. A method for increasing circulating pyrophosphate (PPi) in a subject with ABCC6 Deficiency or ABCC6 deficient subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.8 mg per kilogram of the subject, to thereby increase circulating PPi in the subject.
22. A method for increasing pyrophosphatase activity in a ABCC6 deficient subject or a subject with ABCC6 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby increase pyrophosphatase activity in the subject.
23. A method for ameliorating one or more symptoms of ABCC6 deficient subject or ABCC6 Deficiency in a subject, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby ameliorate one or more symptoms of ABCC6 Deficiency in the subject.
24. A method for treating a ABCC6 deficient subject or a subject with ABCC6 Deficiency, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat the subject.
25. A method of treating a subject afflicted with one or more symptoms of PXE, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat said subject.
26. A method of treating a subject who is an infant and exhibits abnormal vascular calcification and/or one or more symptoms of ENPP1 deficiency, and/or GACI, the method comprising: administering to the subject an ENPP1 agent at a dose of about 0.2 mg per kilogram of the subject, or about 0.4 mg per kilogram of the subject, or about 0.6 mg per kilogram of the subject, or about 1.2 mg per kilogram of the subject, or about 1.8 mg per kilogram of the subject, to thereby treat said subject.
27. The method of any one of claims 1-26, wherein the dose is about 0.2 mg per kilogram of the subject.
28. The method of any one of claims 1-26, wherein the dose is about 0.6 mg per kilogram of the subject.
29. The method of any one of claims 1-26, wherein the dose is about 1.8 mg per kilogram of the subject.
30. The method of any one of claims 1-29, wherein the ENPP1 agent is administered at least one time per week.
31. The method of any one of claims 1-30, wherein the ENPP1 agent is administered at least two times per week.
32. The method of any one of claims 1-30, wherein the ENPP1 agent is administered to the subject at least two times per week following an initial dose.
33. The method of any one of claims 1-32, wherein the ENPP1 agent is administered subcutaneously.
34. The method of any one of claims 1-33, wherein the ENPP1 agent is self-administered.
35. The method of any one of claims 1-34, wherein the ENPP1 agent is administered under a dosing regimen comprising: (a) an initial dose of about 0.2 mg per kilogram of the subject, about 0.6 mg per kilogram of the subject, or about 1.8 mg per kilogram of the subject and (b) about seven days after the initial dose, twice weekly administration of maintenance doses of the ENPP1 agent of about 0.2 mg per kilogram of the subject, about 0.6 mg per kilogram of the subject, or about 1.8 mg per kilogram of the subject.
36. The method of claim 35, wherein the initial dose and the maintenance doses are the same.
37. The method of any one of claims 1-34, wherein the ENPP1 agent comprises the catalytic domain of ENPP1.
38. The method of any one of claims 1-37, wherein the ENPP1 agent comprises the nuclease domain of ENPP1.
39. The method of any one of claims 1-38, wherein the ENPP1 agent comprises the extracellular domain of ENPP1.
40. The method of any one of claims 1-39, wherein the ENPP1 agent comprises a heterologous moiety.
41. The method of claim 40, wherein the heterologous moiety is a polypeptide.
42. The method of claim 40 or 41, wherein the heterologous moiety increases the circulating half-life of the ENPP1 agent relative to the circulating half-life of the ENPP1 agent lacking the heterologous moiety.
43. The method of claim 41 or 42, wherein the heterologous moiety comprises the Fc region of an immunoglobulin molecule.
44. The method of claim 43, wherein the immunoglobulin molecule is a human immunoglobulin molecule.
45. The method of claim 43 or 44, wherein the immunoglobulin molecule is an IgG1.
46. The method of claim 41 or 42, wherein the heterologous moiety comprises albumin.
47. The method of any one of claims 1-35, wherein the ENPP1 agent comprises amino acid residues 99 (PSCAKE) to 925 (QED) of SEQ ID NO:1 [ENPP1 ECD].
48. The method of any one of claims 1-35, wherein the ENPP1 agent comprises amino acid residues 1 (FTAGLKPSCAKE) to 833 (QED) of SEQ ID NO:3.
49. The method of any one of claims 1-35, wherein the ENPP1 agent comprises the amino acid sequence depicted in SEQ ID NO:5
50. The method of any one of claims 1-35, wherein the ENPP1 agent comprises the amino acid sequence depicted in SEQ ID NO:3.
51. The method of any one of claims 1-35, wherein the ENPP1 agent comprises the amino acid sequence depicted in SEQ ID NO:2.
52. The method of any one of claims 1-35, wherein the ENPP1 agent comprises the amino acid sequence depicted in SEQ ID NO:3 or 4 or 5.
53. The method of any one of claims 1-35, wherein the subject has or is suspected of having generalized arterial calcification of infancy (GACI).
54. The method of any one of claims 1-35, wherein the subject has or is suspected of having autosomal recessive hypophosphatemic rickets type 2 (ARHR2).
55. The method of any one of claims 1-35, wherein the pharmaceutical composition contains as the sole active ingredient around 0.2 to 1.8 mg of ENPP1-Fc per kg of weight of the subject.
56. The method of anyone of claims 1-35, wherein the subject has been treated with one or more statins and/or one or more proprotein convertase subtilisin/kexin type 9 (PCSK9) inhibitors prior to said administration of said ENPP1 agent.
57. The method of anyone of claims 1-35, wherein the subject has been treated with a selected dose or selected doses of one or more statins and/or one or more PCSK9 inhibitors for 3 or more years prior administration of said ENPP1 agent.
58. The method of anyone of claims 1-35, wherein the subject has not been diagnosed with a malignancy prior to said administration of said ENPP1 agent.
59. The method of anyone of claims 1-35, wherein the subject has not been diagnosed with a malignancy within the previous 5 years of said administration of said ENPP1 agent,
60. The method of claim 58, wherein said malignancy excludes non-melanoma skin cancers and cervical carcinoma in situ.
61. The method of any one of claims 1-52, wherein the subject has or is suspected of having Pseudoxanthoma elasticum (PXE)
62. The method of any one of claims 1-52, wherein the subject exhibits symptoms similar to a person having autosomal recessive hypophosphatemic rickets type 2 (ARHR2) or generalized arterial calcification of infancy (GACI).
63. The method of claim 1-35, wherein the pharmaceutical composition contains as the sole active ingredient around 0.2 to 1.8 mg/kg of weight of subject
64. The method of any one of claims 1-52, wherein the subject has or is suspected of having one or more of kidney and bladder stones, dental pulp stones, gall stones, salivary gland stones, chronic calculous prostatitis, testicular microliths, calcification in hemodialysis patients, atherosclerosis, malacoplakia, scleroderma (systemic sclerosis), calcinosis cutis, calcific aortic stenosis, calcific tendonitis, synovitis and arthritis, diffuse interstitial skeletal hyperostosis, juvenile dermatomyositis, Generalized Arterial Calcification of Infancy (GACI), Ossification of the Posterior Longitudinal Ligament (OPLL), autosomal hypophosphatemic rickets (ARHR2), osteoarthritis, calcification of atherosclerotic plaques, Chronic Kidney Disease (CKD), End Stage Renal Disease (ESRD), Pseudoxanthoma elasticum (PXE), ankylosing spondylitis, hardening of the arteries, calciphylaxis, and systemic lupus erythematosus.
65. The method of any one of claims 1-52, wherein said subject exhibits calcification of soft connective tissues.
66. The method of claim 65, wherein said subject exhibits accumulation of deposits of calcium and other minerals (mineralization) in elastic fibers of connective tissues.
67. The method of claim 65, wherein said subject exhibits calcification in the eyes, cardiovascular system, and/or skin.
68. The method of claim 67, wherein said subject exhibits narrowing of the arteries and/or arteriosclerosis.
69. The method of treating a subject according to any one of claims 1-52, wherein said subject exhibits ectopic calcification and/or narrow of blood vessels in the lower extremities, and/or claudication.
70. The method of treating a subject according to any one of claims 1-52, wherein said subject exhibits abnormalities in the eyes,
71. The method of claim 70, wherein said eye abnormalities comprising abnormal pigmentation of cells of the retina, and/or angioid streaks, and/or abnormalities in the elastic membrane beneath the retina.
72. The method of treating a subject according to any one of claims 1-52, wherein said subject exhibits choroidal neovascularization
73. The method of treating a subject according to any one of claims 1-52, wherein said subject exhibits visual impairment, vision loss, and/or blindness and also exhibits one or more symptoms of soft tissue calcification.

### INCORPORATION BY REFERENCE

All publications and patents mentioned herein are hereby incorporated by reference in their entirety as if each individual publication or patent was specifically and individually indicated to be incorporated by reference.

### Other Embodiments

While specific embodiments of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1-27. (canceled)

28. A method of reducing or preventing progression of a disease caused by lower levels of plasma PPi in a subject in need thereof, the method comprising: administering to the subject an ENPP1 agent at a dose of about 0.2 mg per kilogram of the subject, or about 0.6 mg per kilogram of the subject, or about 1.8 mg per kilogram of the subject, once a week, to increase circulating PPi levels thereby treating said disease in the subject.

29. The method of claim 28, wherein the ENPP1 agent is administered subcutaneously.

30. The method of claim 28, wherein the ENPP1 agent comprises a heterologous moiety, and wherein said heterologous moiety increases the circulating half-life of the ENPP1 agent relative to the circulating half-life of the ENPP1 agent lacking the heterologous moiety.

31. The method of claim 28, wherein the ENPP1 agent comprises a heterologous moiety and wherein said heterologous moiety comprises the Fc region of an immunoglobulin molecule.

32. The method of claim 28, wherein the ENPP1 agent comprises amino acid residues 99 (PSCAKE) to 925 (QED) of SEQ ID NO: 1 or residues 1 (FTAGLKPSCAKE) to 833 (QED) of SEQ ID NO:3.

33. The method of claim 28, wherein the ENPP1 agent comprises the amino acid sequence depicted in SEQ ID NO: 2 or 3 or 4 or 5.

34. The method of claim 28, wherein the subject has or is suspected of having one or more of kidney and bladder stones, dental pulp stones, gall stones, salivary gland stones, chronic calculous prostatitis, testicular microliths, calcification in hemodialysis, atherosclerosis, malacoplakia, scleroderma (systemic sclerosis), calcinosis cutis, calcific aortic stenosis, calcific tendonitis, synovitis and arthritis, diffuse interstitial skeletal hyperostosis juvenile dermatomyositis, Generalized Arterial Calcification of Infancy (GACI), Ossification of the Posterior Longitudinal Ligament (OPLL), autosomal hypophosphatemic rickets (ARHR2), osteoarthritis, calcification of atherosclerotic plaques, Chronic Kidney Disease (CKD), End Stage Renal Disease (ESRD), Pseudoxanthoma elasticum (PXE), ankylosing spondylitis, hardening of the arteries, calciphylaxis, and systemic lupus erythematosus.

35. The method of claim 28, wherein said subject exhibits one or more of calcification of soft connective tissues, accumulation of deposits of calcium and other minerals (mineralization) in elastic fibers of connective tissues, calcification in the eyes, calcification of cardiovascular system, calcification of skin, narrowing of the arteries, arteriosclerosis, ectopic calcification, narrowing of blood vessels in the lower extremities, claudication, abnormal pigmentation of cells of the retina, angioid streaks, abnormalities in the elastic membrane beneath the retina, choroidal neovascularization, visual impairment, vision loss, and blindness.

36. The method of claim 28, wherein said subject has plasma PPi concentration of below 1300 nM prior to said administration of ENPP1 agent.

37. A method of treating ENPP1 deficiency in a subject in need thereof, the method comprising: administering to the subject an ENPP1 agent at a dose of about 0.2 mg per kilogram of the subject, or about 0.6 mg per kilogram of the subject, or about 1.8 mg per kilogram of the subject, once a week, to reduce pathological calcification thereby treating ENPP1 deficiency in the subject.

38. The method of claim 37, wherein the ENPP1 agent is administered subcutaneously.

39. The method of claim 37, wherein the ENPP1 agent comprises a heterologous moiety, and wherein said heterologous moiety increases the circulating half-life of the ENPP1 agent relative to the circulating half-life of the ENPP1 agent lacking the heterologous moiety.

40. The method of claim 37, wherein the ENPP1 agent comprises a heterologous moiety and wherein said heterologous moiety comprises the Fc region of an immunoglobulin molecule.

41. The method of claim 37, wherein the ENPP1 agent comprises amino acid residues 99 (PSCAKE) to 925 (QED) of SEQ ID NO: 1 or residues 1 (FTAGLKPSCAKE) to 833 (QED) of SEQ ID NO:3.

42. The method of claim 37, wherein the ENPP1 agent comprises the amino acid sequence depicted in SEQ ID NO: 2 or 3 or 4 or 5.

43. The method of claim 37, wherein the subject has or is suspected of having one or more of kidney and bladder stones, dental pulp stones, gall stones, salivary gland stones, chronic calculous prostatitis, testicular microliths, calcification in hemodialysis, atherosclerosis, malacoplakia, scleroderma (systemic sclerosis), calcinosis cutis, calcific aortic stenosis, calcific tendonitis, synovitis and arthritis, diffuse interstitial skeletal hyperostosis juvenile dermatomyositis, Generalized Arterial Calcification of Infancy (GACI), Ossification of the Posterior Longitudinal Ligament (OPLL), autosomal hypophosphatemic rickets (ARHR2), osteoarthritis, calcification of atherosclerotic plaques, Chronic Kidney Disease (CKD), End Stage Renal Disease (ESRD), ankylosing spondylitis, hardening of the arteries, calciphylaxis, and systemic lupus erythematosus.

44. The method of claim 37, wherein said subject exhibits one or more of calcification of soft connective tissues, accumulation of deposits of calcium and other minerals (mineralization) in elastic fibers of connective tissues, calcification in the eyes, calcification of cardiovascular system, calcification of skin, narrowing of the arteries, arteriosclerosis, ectopic calcification, narrowing of blood vessels in the lower extremities, claudication, abnormal pigmentation of cells of the retina, angioid streaks, abnormalities in the elastic membrane beneath the retina, choroidal neovascularization, visual impairment, vision loss, and blindness.

45. The method of claim 37, wherein said subject has plasma PPi concentration of below 1300 nM prior to said administration of ENPP1 agent.

46. A method of treating ABCC6 deficiency in a subject in need thereof, the method comprising: administering to the subject an ENPP1 agent at a dose of about 0.2 mg per kilogram of the subject, or about 0.6 mg per kilogram of the subject, or about 1.8 mg per kilogram of the subject, once a week, to reduce pathological calcification thereby treating ABCC6 deficiency in the subject.

47. The method of claim 46, wherein the ENPP1 agent is administered subcutaneously.

48. The method of claim 46, wherein the ENPP1 agent comprises a heterologous moiety, and wherein said heterologous moiety increases the circulating half-life of the ENPP1 agent relative to the circulating half-life of the ENPP1 agent lacking the heterologous moiety.

49. The method of claim 46, wherein the ENPP1 agent comprises a heterologous moiety and wherein said heterologous moiety comprises the Fc region of an immunoglobulin molecule.

50. The method of claim 46, wherein the ENPP1 agent comprises amino acid residues 99 (PSCAKE) to 925 (QED) of SEQ ID NO: 1 or residues 1 (FTAGLKPSCAKE) to 833 (QED) of SEQ ID NO:3.

51. The method of claim 46, wherein the ENPP1 agent comprises the amino acid sequence depicted in SEQ ID NO: 2 or 3 or 4 or 5.

52. The method of claim 46, wherein the subject has or is suspected of having one or more of kidney and bladder stones, dental pulp stones, gall stones, salivary gland stones, chronic calculous prostatitis, testicular microliths, calcification in hemodialysis, atherosclerosis, malacoplakia, scleroderma (systemic sclerosis), calcinosis cutis, calcific aortic stenosis, calcific tendonitis, synovitis and arthritis, diffuse interstitial skeletal hyperostosis juvenile dermatomyositis, Ossification of the Posterior Longitudinal Ligament (OPLL), autosomal hypophosphatemic rickets (ARHR2), osteoarthritis, calcification of atherosclerotic plaques, Chronic Kidney Disease (CKD), End Stage Renal Disease (ESRD), ankylosing spondylitis, hardening of the arteries, calciphylaxis, and systemic lupus erythematosus.

53. The method of claim 46, wherein said subject exhibits one or more of calcification of soft connective tissues, accumulation of deposits of calcium and other minerals (mineralization) in elastic fibers of connective tissues, calcification in the eyes, calcification of cardiovascular system, calcification of skin, narrowing of the arteries, arteriosclerosis, ectopic calcification, narrowing of blood vessels in the lower extremities, claudication, abnormal pigmentation of cells of the retina, angioid streaks, abnormalities in the elastic membrane beneath the retina, choroidal neovascularization, visual impairment, vision loss, and blindness.

54. The method of claim 46, wherein said subject has plasma PPi concentration of below 1300 nM prior to said administration of ENPP1 agent.


SEQUENCE LISTING (115 sequences)

SEQ ID NO: 1 - protein, 925 aa, Homo sapiens
MERDGCAGGGSRGGEGGRAPREGPAGNGRDRGRSHAAEAPGDPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 2 - protein, 827 aa, synthetic construct
  REGION 1..827: note = ENPP1 Polypeptide
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQED

SEQ ID NO: 3 - protein, 1063 aa, synthetic construct
  REGION 1..1063: note = ENPP1 Polypeptide
FTAGLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 1060 aa, synthetic construct
  REGION 1..1060: note = ENPP1 Polypeptide
GLKPSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 1057 aa, synthetic construct
  REGION 1..1057: note = ENPP1 Polypeptide
PSCAKEVKSCKGRCFERTFGNCRCDAACVELGNCCLDYQETCIEPEHIWTCNKFRCGEKRLTRSLCACSDDCKDKGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDGMVGMLMDGLKELNLHRCLNLILISDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNVFSNMQALFVGYGPGFKHGIEADTFENIEVYNLMCDLLNLTPAPNNGTHGSLNHLLKNPVYTPKHPKEVHPLVQCPFTRNPRDNLGCSCNPSILPIEDFQTQFNLTVAEEKIIKHETLPYGRPRVLQKENTICLLSQHQFMSGYSQDILMPLWTSYTVDRNDSFSTEDFSNCLYQDFRIPLSPVHKCSFYKNNTKVSYGFLSPPQLNKNSSGIYSEALLTTNIVPMYQSFQVIWRYFHDTLLRKYAEERNGVNVVSGPVFDFDYDGRCDSLENLRQKRRVIRNQEILIPTHFFIVLTSCKDTSQTPLHCENLDTLAFILPHRTDNSESCVHGKHDSSWVEELLMLHRARITDVEHITGLSFYQQRKEPVSDILKLKTHLPTFSQEDLINDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 227 aa, synthetic construct
  REGION 1..227: note = human IgG1 Fc
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 906 aa, Mus musculus
MERDGDQAGHGPRHGSAGNGRELESPAAASLLAPMDLGEEPLEKAERARPAKDPNTYKVLSLVLSVCVLTTILGCIFGLKPSCAKEVKSCKGRCFERTFSNCRCDAACVSLGNCCLDFQETCVEPTHIWTCNKFRCGEKRLSRFVCSCADDCKTHNDCCINYSSVCQDKKSWVEETCESIDTPECPAEFESPPTLLFSLDGFRAEYLHTWGGLLPVISKLKNCGTYTKNMRPMYPTKTFPNHYSIVTGLYPESHGIIDNKMYDPKMNASFSLKSKEKFNPLWYKGQPIWVTANHQEVKSGTYFWPGSDVEIDGILPDIYKVYNGSVPFEERILAVLEWLQLPSHERPHFYTLYLEEPDSSGHSHGPVSSEVIKALQKVDRLVGMLMDGLKDLGLDKCLNLILISDHGMEQGSCKKYVYLNKYLGDVNNVKVVYGPAARLRPTDVPETYYSFNYEALAKNLSCREPNQHFRPYLKPFLPKRLHFAKSDRIEPLTFYLDPQWQLALNPSERKYCGSGFHGSDNLFSNMQALFIGYGPAFKHGAEVDSFENIEVYNLMCDLLGLIPAPNNGSHGSLNHLLKKPIYNPSHPKEEGFLSQCPIKSTSNDLGCTCDPWIVPIKDFEKQLNLTTEDVDDIYHMTVPYGRPRILLKQHRVCLLQQQQFLTGYSLDLLMPLWASYTFLSNDQFSRDDFSNCLYQDLRIPLSPVHKCSYYKSNSKLSYGFLTPPRLNRVSNHIYSEALLTSNIVPMYQSFQVIWHYLHDTLLQRYAHERNGINVVSGPVFDFDYDGRYDSLEILKQNSRVIRSQEILIPTHFFIVLTSCKQLSETPLECSALESSAYILPHRPDNIESCTHGKRESSWVEELLTLHRARVTDVELITGLSFYQDRQESVSELLRLKTHLPIFSQED

SEQ ID NO: 8 - protein, 924 aa, Bos taurus
MERDSCAGGGSRGGEGGRGPREGLAGNGRDPGPGRAAEASGEPQAAASLLAPMDLGEEPLERAARARPAKDPNTYKVLSLVLSVCVLTTILGCIFGLKPSCAKEIKSCKGRCFERTFGNCRCDAACVDLGNCCLDYQETCIEPERIWTCTKFRCGEKRLSRSLCSCSDDCKDKGDCCINHGSVCRGEKSWAEEECDSIDEPQCPAGFETPPTLLFSLDGFRAEYLHTWGGLLPVISKLKTCGTYTKNMRPVYPTKTFPNHYSIVTGLYPESHGIIDNNIYDPQMNANFALKNKEKFNPEWYKGEPIWLTAKYQGLKTGTFFWPGSDVKINGIFPDIYKIYNVSVPFEERILAILKWLQLPKDERPHFYTLYLEEPDSSGHSYGPVSSEVIRALQRVDNMVGMLMDGLKELNLHRCLNLILISDHGMEQGSCKKYVYLNKYLGDTKDYKVVYGPAARLRPSDVPDKYYSFDYEGIAKNLSCQEPNQHFKPYLKHFLPKRLHFAKNDRIERLTFYLDPQWQLALNPSERKYCGGGFHGSDNTFLNMQALFIGYGPGFKHSTEVDSFENIEVYNLMCDLLNLTPAPNNGTHGSLNHLLSNPVYTPKHPKEVRPLVQCPFTRAPRESLDCSCDPSILPIVDFQTQLNLTMAEEKTIKRGALPYGRPRVLQNSTVCLLYQHQFVSGYSRDILMPLWTSYTIGRNDSFSTEDFSNCLYQDLRIPLSPVHKCSFYKNNAKLSYGLLSPPQLHKGSSQVYSEALLTTNIVPMYQSFQVIWHYLHGTLLQRYAEERNGLNVVSGPVFDSDYDGRYDSLETLKQNSKIIRNLEVLIPTHFFLVLTSCKNTSQTPLQCENLDAMAFILPHKTDNSESCAHGKHESLWVEELLKLHTARITDVEHITGLSFYQERKEPISDILKLKTHLPTFNQED

SEQ ID NO: 9 - protein, 925 aa, Oryctolagus cuniculus
MERDGCAGGGSRGGEGGRAPREGPAGNSRDPGRSHAAEAPGNPQAAASLLAPMDVGEEPLEKAARARTAKDPNTYKVLSLVLSVCVLTTILGCIFGLKPSCAKEVKSCKGRCFERTFGNCRCDAACVELGNCCLDYQETCIEPEHIWTCNKFRCGEKRLTRSLCACSDDCKDQGDCCINYSSVCQGEKSWVEEPCESINEPQCPAGFETPPTLLFSLDGFRAEYLHTWGGLLPVISKLKKCGTYTKNMRPVYPTKTFPNHYSIVTGLYPESHGIIDNKMYDPKMNASFSLKSKEKFNPEWYKGEPIWVTAKYQGLKSGTFFWPGSDVEINGIFPDIYKMYNGSVPFEERILAVLQWLQLPKDERPHFYTLYLEEPDSSGHSYGPVSSEVIKALQRVDNMVGMLMDGLKELNLHRCLNLILVSDHGMEQGSCKKYIYLNKYLGDVKNIKVIYGPAARLRPSDVPDKYYSFNYEGIARNLSCREPNQHFKPYLKHFLPKRLHFAKSDRIEPLTFYLDPQWQLALNPSERKYCGSGFHGSDNIFSNMQALFVGYGPGFKHGIEVDTFENIEVYNLMCDLLNLTPAPNNGTHGSLNHLLKNPVYTPKHPKEVHPLIQCPFTRNPRDNLGCSCNPSILPIEDFQTQFNLTVAEEKNIKHETLPYGRPRVLQKKNTICLLSQHQFMSGYSQDILMPLWTSYTVDRNDSFSTEDFSNCLYQDFRISLSPVHKCSFYKNNTKVSYGFLSPPQLNKNSRGIYSEALLTTNIVPMYQSFQVIWRYFHDTLLRKYAEERNGVNVVSGPVFDFDYDGRYDSLEILRQKRRVIRNQEILIPTHFFIVLTSCKDASQTPLHCENLDTLAFILPHRTDNSESCLHGKHESSWVEELLMLHRARITDVEHITGLSFYQQRKEPVSDILKLKTHLPTFSQED

SEQ ID NO: 10 - protein, 925 aa, Papio hamadryas
MERDGCAGGGSQGGGKGGRGPREGLAGNGRDPSHGQASEAPGDPQAAASLLAPMDLGEEPLEKAAGARPAKDPNTYKVLSLVLSVCVLTTILGCIFGLKPSCAKEVKSCKGRCFERTFGNCRCDVACVDLGNCCLDYQETCIEPERIWTCNKFRCGEKRLSRSLCACSDDCKERGDCCINYSAVCQGEKSWVEETCENINEPQCPEGFEMPPTLLFSLDGFRAEYLHTWGGLLPVISKLKKCGTYAKNMRPVYPTKTFPNHYSIVTGLYPESHGIIDNKMYDPKMNASFSLKSKEKFNPEWYKGEPIWLTAKYQGLRSGTFFWPGSDVKINGIFPDIYKIYNGSVPFEERILAILKWLRLPKDERPHFYTLYLEEPDSSGHSYGPVSSEVIKALQRVDNMVGMLMDGLKELNLHQCLNLILISDHGMEQGSCKKYIYLNKYLGDTKNIKVIYGPAARLRPSDVPEKYYSFNYENIARNLSCREPNQHFKPYLKHFLPKRLHFAKSDRIEPLTFYLDPQWQLALSPSERKYCGSGFHGSDNVFSNMQALFVGYGPGFQHGIEVDSFENIEVYNLMCDLLNLTPAPNNGTHGSLNHLLKNPIYTPKHPKEVQPSVQCPLAGSPRDSLGCSCNPSILPIVDFQTQFNLTTAEEKNINRASLPYGRPRLLQKKSSVCLLYQHQFVSGYSHDVLMPLWTSYTVNRNDSFSTEDFSNCLYQDLRISFSPIHNCSFYKNNAKLSYGFLSPPQLSKDSSQIYSEALLTSNIVPMYQSFQVIWRYFHDTLLQRYAEERNSINVVSGPVFDSDYDGRYDSSEALKRNRRVIRNQEILIPTHFFIVITSCKNTSQTPLQCDNLDPLAFILPHRSDNSESCVHEKRESSWIEELLMMHRARIMDVEHITGLSFYQERKEPVSDILKLKTHLPTVSQE

SEQ ID NO: 11-26 - skipped (16 entries)

SEQ ID NO: 27 - protein, 4 aa, synthetic construct
  REGION 1..4: note = Linker
GGGA

SEQ ID NO: 28 - protein, 4 aa, synthetic construct
  REGION 1..4: note = Linker
GGGS

SEQ ID NO: 29 - protein, 4 aa, synthetic construct
  REGION 1..4: note = Linker
GGGG

SEQ ID NO: 30 - protein, 5 aa, synthetic construct
  REGION 1..5: note = Linker
GGGGA

SEQ ID NO: 31 - protein, 5 aa, synthetic construct
  REGION 1..5: note = Linker
GGGGS

SEQ ID NO: 32 - protein, 5 aa, synthetic construct
  REGION 1..5: note = Linker
GGGGG

SEQ ID NO: 33 - protein, 4 aa, synthetic construct
  REGION 1..4: note = Linker
GGAG

SEQ ID NO: 34 - protein, 4 aa, synthetic construct
  REGION 1..4: note = Linker
GGSG

SEQ ID NO: 35 - protein, 4 aa, synthetic construct
  REGION 1..4: note = Linker
AGGG

SEQ ID NO: 36 - protein, 5 aa, synthetic construct
  REGION 1..5: note = Linker
SGGGG

SEQ ID NO: 37 - protein, 4 aa, synthetic construct
  REGION 1..4: note = Linker
SGGG

SEQ ID NO: 38 - protein, 4 aa, synthetic construct
  REGION 1..4: note = Linker
GAGA

SEQ ID NO: 39 - protein, 4 aa, synthetic construct
  REGION 1..4: note = Linker
GSGS

SEQ ID NO: 40 - protein, 6 aa, synthetic construct
  REGION 1..6: note = Linker
GAGAGA

SEQ ID NO: 41 - protein, 6 aa, synthetic construct
  REGION 1..6: note = Linker
GSGSGS

SEQ ID NO: 42 - protein, 8 aa, synthetic construct
  REGION 1..8: note = Linker
GAGAGAGA

SEQ ID NO: 43 - protein, 8 aa, synthetic construct
  REGION 1..8: note = Linker
GSGSGSGS

SEQ ID NO: 44 - protein, 10 aa, synthetic construct
  REGION 1..10: note = Linker
GAGAGAGAGA

SEQ ID NO: 45 - protein, 10 aa, synthetic construct
  REGION 1..10: note = Linker
GSGSGSGSGS

SEQ ID NO: 46 - protein, 12 aa, synthetic construct
  REGION 1..12: note = Linker
GAGAGAGAGAGA

SEQ ID NO: 47 - protein, 12 aa, synthetic construct
  REGION 1..12: note = Linker
GSGSGSGSGSGS

SEQ ID NO: 48 - protein, 6 aa, synthetic construct
  REGION 1..6: note = Linker
GGAGGA

SEQ ID NO: 49 - protein, 6 aa, synthetic construct
  REGION 1..6: note = Linker
GGSGGS

SEQ ID NO: 50 - protein, 9 aa, synthetic construct
  REGION 1..9: note = Linker
GGAGGAGGA

SEQ ID NO: 51 - protein, 9 aa, synthetic construct
  REGION 1..9: note = Linker
GGSGGSGGS

SEQ ID NO: 52 - protein, 12 aa, synthetic construct
  REGION 1..12: note = Linker
GGAGGAGGAGGA

SEQ ID NO: 53 - protein, 12 aa, synthetic construct
  REGION 1..12: note = Linker
GGSGGSGGSGGS

SEQ ID NO: 54 - protein, 4 aa, synthetic construct
  REGION 1..4: note = Linker
GGAG

SEQ ID NO: 55 - protein, 4 aa, synthetic construct
  REGION 1..4: note = Linker
GGSG

SEQ ID NO: 56 - protein, 4 aa, synthetic construct
  REGION 1..4: note = Linker
GGAG

SEQ ID NO: 57 - protein, 4 aa, synthetic construct
  REGION 1..4: note = Linker
GGSG

SEQ ID NO: 58 - protein, 8 aa, synthetic construct
  REGION 1..8: note = Linker
GGAGGGAG

SEQ ID NO: 59 - protein, 8 aa, synthetic construct
  REGION 1..8: note = Linker
GGSGGGSG

SEQ ID NO: 60 - protein, 12 aa, synthetic construct
  REGION 1..12: note = Linker
GGAGGGAGGGAG

SEQ ID NO: 61 - protein, 12 aa, synthetic construct
  REGION 1..12: note = Linker
GGSGGGSGGGSG

SEQ ID NO: 62 - protein, 5 aa, synthetic construct
  REGION 1..5: note = Linker
GGGGA

SEQ ID NO: 63 - protein, 5 aa, synthetic construct
  REGION 1..5: note = Linker
GGGGS

SEQ ID NO: 64 - protein, 15 aa, synthetic construct
  REGION 1..15: note = Linker
GGGGAGGGGAGGGGA

SEQ ID NO: 65 - protein, 15 aa, synthetic construct
  REGION 1..15: note = Linker
GGGGSGGGGSGGGGS

SEQ ID NO: 66 - protein, 5 aa, synthetic construct
  REGION 1..5: note = Linker
GGGAG

SEQ ID NO: 67 - protein, 6 aa, synthetic construct
  REGION 1..6: note = Linker
GGGAGG

SEQ ID NO: 68 - protein, 7 aa, synthetic construct
  REGION 1..7: note = Linker
GGGAGGG

SEQ ID NO: 69 - skipped

SEQ ID NO: 70 - protein, 5 aa, synthetic construct
  REGION 1..5: note = Linker
TGGGG

SEQ ID NO: 71 - protein, 4 aa, synthetic construct
  REGION 1..4: note = Linker
AAAL

SEQ ID NO: 72 - protein, 4 aa, synthetic construct
  REGION 1..4: note = Linker
AAAK

SEQ ID NO: 73 - protein, 4 aa, synthetic construct
  REGION 1..4: note = Linker
AAAR

SEQ ID NO: 74 - protein, 14 aa, synthetic construct
  REGION 1..14: note = Linker
EGKSSGSGSESKST

SEQ ID NO: 75 - protein, 12 aa, synthetic construct
  REGION 1..12: note = Linker
GSAGSAAGSGEF

SEQ ID NO: 76 - protein, 12 aa, synthetic construct
  REGION 1..12: note = Linker
AEAAAKEAAAKA

SEQ ID NO: 77 - protein, 18 aa, synthetic construct
  REGION 1..18: note = Linker
KESGSVSSEQLAQFRSLD

SEQ ID NO: 78 - protein, 10 aa, synthetic construct
  REGION 1..10: note = Linker
GENLYFQSGG

SEQ ID NO: 79 - protein, 8 aa, synthetic construct
  REGION 1..8: note = Linker
SACYCELS

SEQ ID NO: 80 - protein, 5 aa, synthetic construct
  REGION 1..5: note = Linker
RSIAT

SEQ ID NO: 81 - protein, 17 aa, synthetic construct
  REGION 1..17: note = Linker
RPACKIPNDLKQKVMNH

SEQ ID NO: 82 - protein, 36 aa, synthetic construct
  REGION 1..36: note = Linker
GGSAGGSGSGSSGGSSGASGTGTAGGTGSGSGTGSG

SEQ ID NO: 83 - protein, 17 aa, synthetic construct
  REGION 1..17: note = Linker
AAANSSIDLISVPVDSR

SEQ ID NO: 84 - protein, 36 aa, synthetic construct
  REGION 1..36: note = Linker
GGSGGGSEGGGSEGGGSEGGGSEGGGSEGGGSGGGS

SEQ ID NO: 85 - protein, 5 aa, synthetic construct
  REGION 1..5: note = Linker
EAAAK

SEQ ID NO: 86 - protein, 5 aa, synthetic construct
  REGION 1..5: note = Linker
PAPAP

SEQ ID NO: 87-90 - skipped (4 entries)

SEQ ID NO: 91 - protein, 15 aa, synthetic construct
  REGION 1..15: note = Linker
  REGION 1..15: note = MISC_FEATURE - The sequence can be anywhere from 1-15 amino acids
RRRRRRRRRRRRRRR

SEQ ID NO: 92 - protein, 16 aa, synthetic construct
  REGION 1..16: note = Linker
DSSSEEKFLRRIGRFG

SEQ ID NO: 93 - protein, 12 aa, synthetic construct
  REGION 1..12: note = Linker
EEEEEEEPRGDT

SEQ ID NO: 94 - protein, 12 aa, synthetic construct
  REGION 1..12: note = Linker
APWHLSSQYSRT

SEQ ID NO: 95 - protein, 12 aa, synthetic construct
  REGION 1..12: note = Linker
STLPIPHEFSRE

SEQ ID NO: 96 - protein, 12 aa, synthetic construct
  REGION 1..12: note = Linker
VTKHLNQISQSY

SEQ ID NO: 97 - protein, 15 aa, synthetic construct
  REGION 1..15: note = Linker
  REGION 2..15: note = MISC_FEATURE - The sequence can be anywhere from 2-15 amino acids
EEEEEEEEEEEEEEE

SEQ ID NO: 98 - protein, 7 aa, synthetic construct
  REGION 1..7: note = Linker
RSGSGGS

SEQ ID NO: 99 - protein, 15 aa, synthetic construct
  REGION 1..15: note = Linker
  REGION 2..15: note = MISC_FEATURE - The sequence can be anywhere from 2-15 amino acids
DDDDDDDDDDDDDDD

SEQ ID NO: 100 - protein, 16 aa, synthetic construct
  REGION 1..16: note = Linker
LVIMSLGLGLGLGLRK

SEQ ID NO: 101 - protein, 15 aa, synthetic construct
  REGION 1..15: note = Linker
VIMSLGLGLGLGLRK

SEQ ID NO: 102 - protein, 14 aa, synthetic construct
  REGION 1..14: note = Linker
IMSLGLGLGLGLRK

SEQ ID NO: 103 - protein, 13 aa, synthetic construct
  REGION 1..13: note = Linker
MSLGLGLGLGLRK

SEQ ID NO: 104 - protein, 12 aa, synthetic construct
  REGION 1..12: note = Linker
SLGLGLGLGLRK

SEQ ID NO: 105 - protein, 11 aa, synthetic construct
  REGION 1..11: note = Linker
LGLGLGLGLRK

SEQ ID NO: 106 - protein, 10 aa, synthetic construct
  REGION 1..10: note = Linker
GLGLGLGLRK

SEQ ID NO: 107 - protein, 9 aa, synthetic construct
  REGION 1..9: note = Linker
LGLGLGLRK

SEQ ID NO: 108 - protein, 8 aa, synthetic construct
  REGION 1..8: note = Linker
GLGLGLRK

SEQ ID NO: 109 - protein, 7 aa, synthetic construct
  REGION 1..7: note = Linker
LGLGLRK

SEQ ID NO: 110 - protein, 6 aa, synthetic construct
  REGION 1..6: note = Linker
GLGLRK

SEQ ID NO: 111 - protein, 5 aa, synthetic construct
  REGION 1..5: note = Linker
LGLRK

SEQ ID NO: 112 - protein, 4 aa, synthetic construct
  REGION 1..4: note = Linker
GLRK

SEQ ID NO: 113-114 - skipped (2 entries)

SEQ ID NO: 115 - protein, 15 aa, synthetic construct
  REGION 1..15: note = Linker
  REGION 2..15: note = MISC_FEATURE - The sequence can be anywhere from 2-15 amino acids
KKKKKKKKKKKKKKK